SEQUENCE LISTING (87 sequences)

SEQ ID NO: 1 - protein, 124 aa, Anaplasma phagocytophilum
MIPLAPWKSISVVYMSGSDEYKEIIKQCIGSVKEVFGEGRFDDVVASIMKMQEKVLASSMQQDDTGTVGQIESGEGSGARLSDEQVQQLMNSIREEFKDDLRAIKRRILKLERAVYGANTPKES

SEQ ID NO: 2 - protein, 24 aa, Anaplasma phagocytophilum
LRAIKRRILKLERAVYGANTPKES

SEQ ID NO: 3 - protein, 12 aa, Anaplasma phagocytophilum
RAVYGANTPKES

SEQ ID NO: 4 - protein, 205 aa, Anaplasma phagocytophilum
MLRRSSFFCLLALLSVTSCGTLLPDSNVGVGRHDLGSHRSVAFAKKVEKVYFDIGKYDLKGPGKKVILELVEQLRQDDSMYLVVIGHADATGTEEYSLALGEKRANAVKQFIIGCDKSLAPRVTTQSRGKAEPEVLVYSTDAQEVEKANAQNRRAVIVVEFAHIPRSGVADMHAPVASSITSENSNASAEGEDMEASEFSSAIAN

SEQ ID NO: 5 - protein, 57 aa, Anaplasma phagocytophilum
CGTLLPDSNVGVGRHDLGSHRSVAFAKKVEKVYFDIGKYDLKGPGKKVILELVEQLR

SEQ ID NO: 6 - protein, 16 aa, Anaplasma phagocytophilum
LKGPGKKVILELVEQL

SEQ ID NO: 7 - protein, 9 aa, Anaplasma phagocytophilum
EKVYFDIGK

SEQ ID NO: 8 - protein, 16 aa, Anaplasma phagocytophilum
GHADATGTEEYSLALG

SEQ ID NO: 9 - protein, 21 aa, Anaplasma phagocytophilum
LVYSTDAQEVEKANAQNRRAV

SEQ ID NO: 10 - protein, 149 aa, Anaplasma phagocytophilum
PDSNVGVGRHDLGSHRSVAFAKKVEKVYFDIGKYDLKGPGKKVILELVEQLRQDDSMYLVVIGHADATGTEEYSLALGEKRANAVKQFIIGCDKSLAPRVTTQSRGKAEPEVLVYSTDAQEVEKANAQNRRAVIVVEFAHIPRSGVADM

SEQ ID NO: 11 - protein, 12 aa, Anaplasma phagocytophilum
LRAIKRRILKLE

SEQ ID NO: 12 - protein, 42 aa, Anaplasma phagocytophilum
DEYKEIIKQCIGSVKEVFGEGRFDDVVASIMKMQEKVLASSM

SEQ ID NO: 13 - protein, 130 aa, Anaplasma marginale
MSGEDEYKEIIRQCIGSVKEVFGEGRFDDVVASIMKMQEKVLASSMKDGDPVGQIAADGVGNELYDRIADRLEERVSQKISEDLRIIKKRLLRLERVVLGGGSVSGDAAAHQVSGNQPSQQNSSAAAEGG

SEQ ID NO: 14 - protein, 32 aa, Anaplasma marginale
LGGGSVSGDAAAHQVSGNQPSQQNSSAAAEGG

SEQ ID NO: 15 - protein, 131 aa, Anaplasma marginale, subspecies Centrale
MSGEDEYKEIIRQCIGSVKEVFGEGRFDDVVASIMKMQEKVLASSMKDGDPVGQIAADGVGNELYDRIADRLEERVSQKISEDLRIIKKRLLRLERVVLGGGSVSGDAAAAHQVSGNQPSQQNSSAAAEGG

SEQ ID NO: 16 - protein, 33 aa, Anaplasma marginale, subspecie Centrale
LGGGSVSGDAAAAHQVSGNQPSQQNSSAAAEGG

SEQ ID NO: 17 - protein, 46 aa, Anaplasma marginale and A. marginale subspecies Centrale
MSGEDEYKEIIRQCIGSVKEVFGEGRFDDVVASIMKMQEKVLASSM

SEQ ID NO: 18 - protein, 16 aa, Anaplasma marginale and A. marginale subspecies Centrale
DLRIIKKRLLRLERVV

SEQ ID NO: 19 - protein, 104 aa, Ehrlichia chaffeensis
MAEDDYKGVIKQYIDTVKEIVGDSKTFDQMFESVVRIQERVMAANAQNNEDGVIDNGDQVKRIGSSTSESISNTEYKELMEELKVIKKRILRLERKILKPKEEV

SEQ ID NO: 20 - protein, 42 aa, Ehrlichia chaffeensis
MAEDDYKGVIKQYIDTVKEIVGDSKTFDQMFESVVRIQERVM

SEQ ID NO: 21 - protein, 13 aa, Ehrlichia chaffeensis
ELKVIKKRILRLE

SEQ ID NO: 22 - protein, 10 aa, Ehrlichia chaffeensis
RKILKPKEEV

SEQ ID NO: 23 - protein, 98 aa, Ehrlichia canis
MADDEYKGVIQQYINTVKEIVSDSKTFDQMFESVVKIQERVMEANAQNDDGSQVKRIGSSTSDSISDSQYKELIEELKVIKKRLLRLEHKVLKPKEGA

SEQ ID NO: 24 - protein, 42 aa, Ehrlichia canis
MADDEYKGVIQQYINTVKEIVSDSKTFDQMFESVVKIQERVM

SEQ ID NO: 25 - protein, 13 aa, Ehrlichia canis
ELKVIKKRLLRLE

SEQ ID NO: 26 - protein, 10 aa, Ehrlichia canis
HKVLKPKEGA

SEQ ID NO: 27 - protein, 105 aa, Ehrlichia ruminantium
MADEDYKGVIKQYIDTVKEIVGDSKTFDQMFESVVKIQERVMAASAQNEANGALVEGDSKMKRIRSADDSIAYTQSQELLEELKVLKKRIARLERHVFKSNKTEA

SEQ ID NO: 28 - protein, 42 aa, Ehrlichia ruminantium
MADEDYKGVIKQYIDTVKEIVGDSKTFDQMFESVVKIQERVM

SEQ ID NO: 29 - protein, 13 aa, Ehrlichia ruminantium
ELKVLKKRIARLE

SEQ ID NO: 30 - protein, 11 aa, Ehrlichia ruminantium
RHVFKSNKTEA

SEQ ID NO: 31 - protein, 236 aa, Anaplasma marginale
MLHRWLALCFLASFAVTGCGLFSKEKVGMDIVGVPFSAGRVEKVYFDFNKYEIKGSGKKVLLGLVERMKADKRSTLLIIGHTDSRGTEEYNLALGERRANAVKEFILGCDRSLSPRISTQSRGKAEPEVLVYSSDFKEAEKAHAQNRRVVLIVECQHSVSPKKKMAIKWPFSFGRSAAKQDDVGSSEVSDENPVDDSSEGIASEEAAPEEGVVSEEAAEEAPEVAQDSSAGVVAPE

SEQ ID NO: 32 - protein, 59 aa, Anaplasma marginale
LFSKEKVGMDIVGVPFSAGRVEKVYFDFNKYEIKGSGKKVLLGLVERMKADKRSTLLII

SEQ ID NO: 33 - protein, 212 aa, Anaplasma marginale, subspecies Centrale
MLHRWLALCLLASLAVTGCELFNKEKVNIDIGGVPLSAGRVEKVYFDFNKYEIKGSGKKVLLGLVERMKADKMSTLLIVGHTDSRGTEEYNLALGERRANAVKEFILGCDRSLSPRISTQSRGKAEPEILVYSSDFKEAEKAHAQNRRVVLIMECQHAASPKKARVSRWPFSFGRSSATQQDNGGGTVAAGSPGEDAPAEVVEPEETQEAGE

SEQ ID NO: 34 - protein, 59 aa, Anaplasma marginale, subspecies Centrale
LFNKEKVNIDIGGVPLSAGRVEKVYFDFNKYEIKGSGKKVLLGLVERMKADKMSTLLIV

SEQ ID NO: 35 - protein, 34 aa, Anaplasma marginale and A. marginale subspecies Centrale
AGRVEKVYFDFNKYEIKGSGKKVLLGLVERMKAD

SEQ ID NO: 36 - protein, 16 aa, Anaplasma marginale and A. marginale subspecies Centrale
GHTDSRGTEEYNLALG

SEQ ID NO: 37 - protein, 30 aa, Anaplasma marginale and A. marginale subspecies Centrale
RRANAVKEFILGCDRSLSPRISTQSRGKAE

SEQ ID NO: 38 - protein, 23 aa, Anaplasma marginale and A. marginale subspecies Centrale
LVYSSDFKEAEKAHAQNRRVVLI

SEQ ID NO: 39 - protein, 175 aa, Ehrlichia chaffeensis
MKHKLVFIKFMLLCLILSSCKTTDHVPLVNVDHVFSNTKTIEKIYFGFGKATIEDSDKTILEKVMQKAEEYPDTNIIIVGHTDTRGTDEYNLELGKQRANAVKDFILERNKSLEDRIIIESKGKSEPAVLVYSNNPEEAEYAHTKNRRVVITLTDNLIYKAKSSDKDPSSNKTEQ

SEQ ID NO: 40 - protein, 50 aa, Ehrlichia chaffeensis
NVDHVFSNTKTIEKIYFGFGKATIEDSDKTILEKVMQKAEEYPDTNIIIV

SEQ ID NO: 41 - protein, 27 aa, Ehrlichia chaffeensis
IEDSDKTILEKVMQKAEEYPDTNIIIV

SEQ ID NO: 42 - protein, 17 aa, Ehrlichia chaffeensis
GHTDTRGTDEYNLELGE

SEQ ID NO: 43 - protein, 33 aa, Ehrlichia chaffeensis
QRANAVKDFILERNKSLEDRIIIESKGKSEPAV

SEQ ID NO: 44 - protein, 22 aa, Ehrlichia chaffeensis
LVYSNNPEEAEYAHTKNRRVVI

SEQ ID NO: 45 - protein, 162 aa, Ehrlichia canis
MKHKLVFIKFILLCLILSSCKTTDHVPLVNTDHVFSNMKTIEKIYFDFGKATIGDSDKAILEKVIQKAQKDTNTNIVIVGHTDTRGTDEYNLELGEQRANAVKDFIIEHDKSLENRITVQSKGKSEPAVLVYSSNPEEAEHAHAKNRRVVITLTDNGNKTSQ

SEQ ID NO: 46 - protein, 58 aa, Ehrlichia canis
TTDHVPLVNTDHVFSNMKTIEKIYFDFGKATIGDSDKAILEKVIQKAQKDTNTNIVIV

SEQ ID NO: 47 - protein, 26 aa, Ehrlichia canis
GDSDKAILEKVIQKAQKDTNTNIVIV

SEQ ID NO: 48 - protein, 17 aa, Ehrlichia canis
GHTDTRGTDEYNLELGE

SEQ ID NO: 49 - protein, 33 aa, Ehrlichia canis
QRANAVKDFIIEHDKSLENRITVQSKGKSEPAV

SEQ ID NO: 50 - protein, 22 aa, Ehrlichia canis
LVYSSNPEEAEHAHAKNRRVVI

SEQ ID NO: 51 - protein, 217 aa, Ehrlichia runantium
MRYQLIVANLILLCLTLNGCHFNSKHVPLVNVHNLFSNIKAIDKVYFDLDKTVIKDSDKVLLEKLVQKAQEDPTTDIIIVGHTDTRGTDEYNLALGEQRANAVRDFIISCDKSLEKRITVRSKGKSEPAILVYSNNPKEAEDAHAKNRRVVITLVNNSTSTDNKVPTTTTPFNEEAHNTISKDQENNTQQQAKSDNINNINTQQKLEQDNNNTPEVN

SEQ ID NO: 52 - protein, 58 aa, Ehrlichia ruminantium
NSKHVPLVNVHNLFSNIKAIDKVYFDLDKTVIKDSDKVLLEKLVQKAQEDPTTDIIIV

SEQ ID NO: 53 - protein, 25 aa, Ehrlichia ruminantium
DSDKVLLEKLVQKAQEDPTTDIIIV

SEQ ID NO: 54 - protein, 17 aa, Erhlichia ruminantium
GHTDTRGTDEYNLALGE

SEQ ID NO: 55 - protein, 33 aa, Ehrlichia ruminantium
QRANAVRDFIISCDKSLEKRITVRSKGKSEPAI

SEQ ID NO: 56 - protein, 22 aa, Ehrlichia ruminantium
LVYSNNPKEAEDAHAKNRRVVI

SEQ ID NO: 57 - protein, 355 aa, Anaplasma phagocytophilum
MSFTMSKLSLDPTQGSHTAENIACSIFDMVLGVKSTAKLLAGTWAGTSSTIWKTVTGAASSTKEASSKSYGTLRSSLGSSASRRMLGTCATAALCLTAPLLGAAAAGAAITCALITICMALLFLVLYTVLHIASQMLRCASLLLSMVCNILHSTFTATKSCLGGKSPARTTEERVAGDLDHKGVDSDRKHDAEKTEEKKHGLGSLCKSLAINLVSLMGTALVTTPIILLAVVLLVLVPVYLLCATVHHIYQGNYEDRNNDKGSSRGGGTTYYPMTMSASASEESLSSIISEGGLSKTSLPSYSAATATGTGNATGEVFSHSHSSGKSSSKPESRPESNLQNVVAETMSQQQRSVS

SEQ ID NO: 58 - protein, 338 aa, Anaplasma phagocytophilum
MRTFCWFVHRFYLRNYCFLNKNCSQCSNTTYTSTTVIIYATISSKSIVISFSDARCVEDFKGKFTTLDAGIASRAIFSMSVAIKYSDKNLVELIPEGEFTYCDVNTMVGHMLRHGFTFKQEVLSSILEQASALATENFVVLKAGERSSYIVGVYQDTVTVSPLTSEYLDLESGPSQRLVKLLRTESAISSVNVDAQNRSITILVRGNVCDALGTLCNVMITIGAIEAKEKGAVLVKLVRLAFLDLMGNEIRSVRNIASCSVAHPLSKYKGVARTIENILTCLSNKTLDAVVLGQLEDALEGKGEFSALPSVLTKGFVKLNRDFNGQLENIIGSEKRVQ

SEQ ID NO: 59 - protein, 147 aa, Anaplasma phagocytophilum
MQQSISTDTLGSSEVRQPKPRKIATGARASRATTAARKSVSSTTNKNVAVDVRSRSSKSHNDDKVAIDSHAEARQLPEEDRKESLSPDVSTVKSEHASRSSEDIQSPVDNSGPEVSGGLKTRYSAWIALLCKQYGRFTAFFSKKRES

SEQ ID NO: 60 - protein, 645 aa, Anaplasma phagocytophilum
MAAERIIGIDLGTTNSCVAVMEAGTAKVIENSEGSRTTPSVVAFTDNERLVGELAKRQANINAQNTIYASKRIIGRRYDDMRDLKCPYEVFPAKNGDAWIRAKGEGYSPVQIGAFVLEKIKETAERYFGAPVKKAVITVPAYFNDAQRQATKDAGTIAGLDVVRIINEPTAAALAYGLDKGDKQRTIVVYDLGGGTFDVSVLEIADGVFEVKATNGDTKLGGEDFDNAIMEHMMESFQKETGINLRNDPMAVQRVKEAAEKAKIELSTRLETDITLPFISSDSTGAKHLSLKLSRAKFEGLVDELIERTIEPCKKALSDAGIKDNSKVDEVVLVGGMTRVPKVIQRVKDFFGKEPCQGVNPDEVVAVGAAIQGGILTGDVRDVLLLDVAPLSLGIETLGGVFTPLIERNTTIPTKKSQVFSTAEDGQTAVTIKVYQGERKMAIDNKLLGQFSLEGIPHAPRGVPQIEVTFDIDANGIVHVSAKDKASGKEQTIKIQSSGGLSDEEIKKMVKDAQDRAEDDEKRKKHVELKNSSEGLIHSVEKSLKDYGDKVAGADKSNIESAIKDLRECLNDSNCSTDTLQQKYDALMNLSMKLGEAAYAANKNDGAGSADQSGSSSGGSDGNPEERVVDSEYQEINKDEDKKNT

SEQ ID NO: 61 - protein, 453 aa, Anaplasma phagocytophilum
MKATLITCYTQVCVCYGYVMHSSMIYNSKTYRVYSRVAGEIKDDRLTHRAVAVYCSWLLERSINELRAVLETSGPDGYVFVQLALDRMEEVYNDIYQARAGTHDDIVKALSANCDQYLFQCRSALFHLSRFRDGSLPLEGPVGDDVSSFCTASSNIASVITLLQTNRSLPDRVSSDTRNRLCMLIDSLSDSVTAMPDSAFMHLAQGSAGFASVYDARCAFLFAVEELRALAYTVHTDTDTAARVCLGDSFEALLENIREAIRRVSDAPGVTARASCSCTLANKALARIQAMFENYVNGTHARDSDLSDEMYMSTTIVSAYAAARSLCYSCISAASELPCVPSIIECSSALYDLYSHLSARAFIDLADPHDVNNILPALNKAREALGKVDRSTLPSNRDTEIYDRLRKAIEQASGRCIMRQLEPDYLDLAPSTGQNDLSIEGLGAAGASHDLHH

SEQ ID NO: 62 - protein, 442 aa, Anaplasma phagocytophilum
MCVCYGAVMHSFIDPISKTYRVYSNVEESLRSGEFTERAVAVRTSWLLEQALERLHRVVEASEEGIPSSLVKMALQNVRDIYSNIYRAREGTANNIKKALVDNGREHISKLRTVLLYLALARSKSLPNEGPAGASVTEISAASYNAASALSILQSNLGLPDEASVNTRDRLCVLLDSLSGTIELIPGRALLRSVRGSTGFISPSEVRNALLLAVEEAHALVYTTHDSADKDARGCVQGALELVLYSIKRVICGIRGKNISSRASWSCALASQMMYTIQEVFDGYVSNTHTRDSDVSDKEFLSNNVIRAFTSARHLLASCVSVPPEERPSSSEYVIRCSGMLREVYSHLGTCESIDLANPHGANNILPALNKAREALDEVDPSDLPSHRDAETYSRIREAIMQASRRCIMQQCSEPDLLDSALGAGWDALSIEGLGAGCWRFS

SEQ ID NO: 63 - protein, 1047 aa, Anaplasma phagocytophilum
MAKRFLNDTEKKLLSLLKSVMQHYKPRTGFVRALLSALRSISVGNPRQTAHDLSVLVTQDFLVEVIGSFSTQAIAPSFLNIMALVDEEALNHYDRPGRAPMFADMLRYAQEQIRRGNLLQHRWNEETFASFADSYLRRRHERVSAEHLRQAMQILHAPASYRVLSTNWFLLRLIAAGYVRNAVDVVDAESAGLTSPRSSSERTAIESLLKDYDEEGLSEMLETEKGVMTSLFGTVLLSTYVNELRAEVAQEFAEHHRFLSRVLSTCSALLSPLGTVAVVAYCASMFSSIIQQATNPSSDKEKYCALIDYINETIASFGSGNGNATITEALIRGSNLTALFGNHSCSESNEALHDILSNRRNESLSITNMSAMPASISVLTTMYLALPIIAFGGYAAQWVSRRMSSRGRGGFSSPEMFSMLSAVVCAKLGLNTFLTLTAHVSHKAFSTALNWSVTRLFLPLSLIEQPKKIGLFVNSAMSAAWSSRRLRFEPSSRACAIAAALSIPFEYAGHVVAKLHVINTGWTQVPPSCRQILNFTVKHARVAAFFGTIIAARRHIRNMPYSRRLERIIWADGVKATTAPAALLLLDVAAGNVFLSTVVLTVDSLVSLIPDMICSANVDMLNNAGNQLAALEQWLVENLDEEALLKIAMLTSLQRLPGSTHGELEKILEEFYNKDQITDHGVDLTVDDDFTEGITERQLLEWQSDDASRRRTRGGDCADASSEGELIGATSRDYYDPPERRRGPTLYEELVRGILERHGTRFSDALAGEEEDADEALLFSDLRLQLDDAAVPHEEQSERGRSSRRGRFCGDEDFDVKCQGQGDGRRSRRSDRRGYSEEPALGDMRHSSRGAASESDARRSRRSDREEPATSPRRHPAGEVPQRQDEASPSGLRNHPSGAIPKVRSASAAMHTKKDKSKKSARSSESTRRGVDLGFLGSPKDLERCVLEGERARARSPRCGVGTPPCHLDRVVYETEGAQDVDNDVFDVSRYVTPRNQAGERVRVGTSSSSRAPQGATGLAPGTSLTSLDDDALDILDAIQGQRGRRR

SEQ ID NO: 64 - protein, 1528 aa, Anaplasma phagocytophilum
MTLLLKQNPPKASVALLGSAIDFFLCRDRNSHPARRRMVILLAEGFTLREGSAVPPALIHENLTSPDLLARALHKTASNSTAFQQVPFQLWHALALAYNSLPGKNQEEDLTNFVLGCLDGVSEDMTIVREEDSTTFEVQSYTTFSRVHSLLASAPSSYKNGALTVHESCIFSIQDKSGVPIAKVKMWVEYDIAPSTKAEGVYRTAVKKVKLVLTERDCRDVRQGEPGSVCSWHNIPKALAKHYVRVPERPTHVLYSACNLQRHNPRYMARRVFYDVSGIDECILRAYSVISGMPPEVLELSFCNTVISQEASGVFRVVVRGVVGLVGYDKSSVVQQGAVSHGRDAVSKMGVCMSFVASQAHDACATILRHVAVTVNTFGNVLTLGGGISLRDFLAGSAKDTDFAGSHICNFGEEIVAHGLSLWEDLGKRHRWASHSVPVRGDCGIFIQHSDEIREILRSQPKHAANIVEKTGVNTENLRVLLSSILSNSSGSSLPVELAAHYVAHEGVVADNGDSARRLPVNQHVLEEHLVYRVTSVSGIHIHACVDYVVEDIDTPGSVKDLGLCIRDVRIGTRVASSAEEVCSAIQEKEGRIDRNDFAWFNVDQSLVETSRAEFRAAIGTLPILPSTRSLLSSDLTYFSRDCSKIENAVKERMLPALTLYAKKIAKRNAEGMLRVTGDPLRGSADTRWLAQMLESGKVLVQSPNILSMEEDGTAFVSPNFNPAKCEEDDVREAGGVRARLAATLQNMLGDPRIHVAISEAIVSMSDVRGTDLVRLCRELICTTMLSKKCAVQVVDTGLRIIPDVQQGGTGTLRLYQHVLFAPVAWWIEKPIAIHLVVRSDLVIHRDLTGALAFNIESVRFGLRASQNTVLSTSALLLECKPSLLGLCCTVDVQPSEEEGVYSSRALHVMAAIQRYYGSAYSFLLVDPLEDTRSTNDSLLLLVRTGIGEFLNVFGVDGVVRHPLLCFTDSSQDVDEHPTSEQDIYNWISKNYPRHGDDIGGIISEALFNATGFGNVCKFLRFSVGPNLEITPVERGGYRNPQDVSGVIASGPDGLFTARPYLVKLRKGSETSTLGLVCTCNISVRPGGNNEILVQVRGLKVSLCSGKNLLKFFLSTSTDQGIYHEQYSEFLHSLEPCSDLSEHCLQARVQSAKLANYVRRKQHPGIHTQHEHAPGGPKASDAGSHTMKRHGRVLPTPMDPKVLQDLRSSNLLAAAFGGERFPENDHILRTMKALVDVASRGQIICASPERGHKGALYTNVARMSENRLWVLHNACFMTPDLRVLMVELHYKVDRKKSPHGGRDIFEICDGSFNVASGDTPSKKDFSIRIPKNVQVSENKWNIFSEMLKPPVVPESFLDKMCRWLTTAWNSLKSFVSNAGGYVMRLFRACCSCVRPQNVSEDNVTLLDSNRDSHECESAVSEVSAPAPVIGTSSEHVHSNDVDTAQSSTKAKGTDGKKPSTTVPKKPPRPARGAKSSSAHSVAGVTQGGAGDVTREVGGPSTSVADPTAASSVSQLQSSRASNVSQQQH

SEQ ID NO: 65 - protein, 416 aa, Anaplasma phagocytophilum
MVCCVSRVVLYIASVILLMLIMGEDASAAIYKDDLPPNSKKFYVALDYAPALSRVSTFDIVGDGKTHIALPYLKNDQEDRFNAEAIDWDAPNLSVQFKNSVLMSWVGSIGYKMMGGRLELEVGHEKFGARVSSGENREENSDVAYVFFSRLLPYYLVSAQYEKLISGLANLTEDEILAFANGVADQRPDLDKKICKKARLGGDDRGTDAQAACRDSIKGADVGGFGAFMRKAIGTYLMWRYNGGSDRYGLERGGRSVNSKDIVSDIKELPKEERKILAGILAAATGYGVVVEIPSVAATSVMVNACYDHNVSLTRKRASAYSCVGLGSTFVEIVDEHRAAKLAYRLKAGLSYNFASGVTAFVGGFYHHIIGDSWYDRVPMRTVFLDEKTGERPVKTGKVDLSLDYIGAECGIRLIL

SEQ ID NO: 66 - protein, 134 aa, Anaplasma phagocytophilum
MKGKSDSEIRTSSSIRTSSSDDSRSSDDSTRIRASKTHPQAPSDNSSILSSEDIESVMRCLEEEYGQKLSSELKKSMREEISTAVPELTRALIPLLASASDSDSSSRKLQEEWVKTFMAIMLPHMQKIVASTQG

SEQ ID NO: 67 - protein, 159 aa, Anaplasma marginale
LFSKEKVGMDIVGVPFSAGRVEKVYFDFNKYEIKGSGKKVLLGLVERMKADKRSTLLIIGHTDSRGTEEYNLALGERRANAVKEFILGCDRSLSPRISTQSRGKAEPEVLVYSSDFKEAEKAHAQNRRVVLIVECQHSVSPKKKMAIKWPFSFGRSAAK

SEQ ID NO: 68 - protein, 157 aa, Anaplasma marginale, subspecies Centrale
NKEKVNIDIGGVPLSAGRVEKVYFDFNKYEIKGSGKKVLLGLVERMKADKMSTLLIVGHTDSRGTEEYNLALGERRANAVKEFILGCDRSLSPRISTQSRGKAEPEILVYSSDFKEAEKAHAQNRRVVLIMECQHAASPKKARVSRWPFSFGRSSAT

SEQ ID NO: 69 - protein, 135 aa, Ehrlichia chaffeensis
NVDHVFSNTKTIEKIYFGFGKATIEDSDKTILEKVMQKAEEYPDTNIIIVGHTDTRGTDEYNLELGKQRANAVKDFILERNKSLEDRIIIESKGKSEPAVLVYSNNPEEAEYAHTKNRRVVITLTDNLIYKAKSS

SEQ ID NO: 70 - protein, 135 aa, Ehrlichia canis
TTDHVPLVNTDHVFSNMKTIEKIYFDFGKATIGDSDKAILEKVIQKAQKDTNTNIVIVGHTDTRGTDEYNLELGEQRANAVKDFIIEHDKSLENRITVQSKGKSEPAVLVYSSNPEEAEHAHAKNRRVVITLTDN

SEQ ID NO: 71 - protein, 195 aa, Ehrlichia ruminantium
NSKHVPLVNVHNLFSNIKAIDKVYFDLDKTVIKDSDKVLLEKLVQKAQEDPTTDIIIVGHTDTRGTDEYNLALGEQRANAVRDFIISCDKSLEKRITVRSKGKSEPAILVYSNNPKEAEDAHAKNRRVVITLVNNSTSTDNKVPTTTTPFNEEAHNTISKDQENNTQQQAKSDNINNINTQQKLEQDNNNTPEVN

SEQ ID NO: 72 - protein, 110 aa, Ehrlichia ruminantium
MCSSFMADEDYKGVIKQYIDTVKEIVGDSKTFDQMFESVVKIQERVMAASAQNEANGALVEGDSKMKRIRSADDSIAYTQSQELLEELKVLKKRIARLERHVFKSNKTEV

SEQ ID NO: 73 - DNA, 37 bp, Artificial Sequence
  Synthetic primer
gatcggatcccttttcagcaaggaaaaggtcgggatg

SEQ ID NO: 74 - DNA, 33 bp, Artificial Sequence
  Synthetic primer
atcggcggccgcctattcaggcgcgaccactcc

SEQ ID NO: 75 - DNA, 32 bp, Artificial Sequence
  Synthetic primer
gacgacgacaaaatgcttttcagcaaggaaaa

SEQ ID NO: 76 - DNA, 31 bp, Artificial Sequence
  Synthetic primer
gaggagaagcccggttactattcaggcgcga

SEQ ID NO: 77 - protein, 16 aa, Anaplasma marginale
IKGSGKKVLLGLVERM

SEQ ID NO: 78 - protein, 16 aa, Artificial Sequence
  Synthetic consensus sequence
  X (1)..(1): X is leucine or isoleucine
  X (4)..(4): X is proline or serine
  X (9)..(9): X is isoleucine or leucine
  X (11)..(11): X is glutamic acid or glycine
  X (15)..(15): X is glutamine or arginine
  X (16)..(16): X is leucine or methionine
XKGXGKKVXLXLVEXX

SEQ ID NO: 79 - protein, 18 aa, Anaplasma marginale
KYEIKGSGKKVLLGLVER

SEQ ID NO: 80 - protein, 18 aa, Anaplasma phagocytophilum
KYDLKGPGKKVILELVEQ

SEQ ID NO: 81 - protein, 18 aa, Artificial Sequence
  Synthetic consensus sequence
  X (3)..(3): X is glutamic acid or aspartic acid
  X (4)..(4): X is leucine or isoleucine
  X (7)..(7): X is proline or serine
  X (12)..(12): X is isoleucine or leucine
  X (14)..(14): X is glutamic acid or glycine
  X (18)..(18): X is glutamine or arginine
KYXXKGXGKKVXLXLVEX

SEQ ID NO: 82 - protein, 19 aa, Anaplasma marginale
KYEIKGSGKKVLLGLVERM

SEQ ID NO: 83 - protein, 19 aa, Anaplasma phagocytophilum
KYDLKGPGKKVILELVEQL

SEQ ID NO: 84 - protein, 19 aa, Artificial Sequence
  Synthetic consensus sequence
  X (3)..(3): X is glutamic acid or aspartic acid
  X (4)..(4): X is leucine or isoleucine
  X (7)..(7): X is proline or serine
  X (12)..(12): X is isoleucine or leucine
  X (14)..(14): X is glutamic acid or glycine
  X (18)..(18): X is glutamine or arginine
  X (19)..(19): X is methionine or leucine
KYXXKGXGKKVXLXLVEXX

SEQ ID NO: 85 - protein, 5 aa, Artificial Sequence
  Synthetic consensus sequence
  X (2)..(2): X is serine or proline
GXGKK

SEQ ID NO: 86 - protein, 5 aa, Anaplasma marginale
GSGKK

SEQ ID NO: 87 - protein, 5 aa, Anaplasma phagocytophilum
GPGKK

(19) United States
(12) Patent Application Publication
(10) Pub. No.: US 2019/0117753 A1
(43) Pub. Date: Apr. 25, 2019

(54) OMPA IN VACCINE COMPOSITIONS AND AS DIAGNOSTIC TARGETS

(71) Applicant: VIRGINIA COMMONWEALTH UNIVERSITY, Richmond, VA (US)

(72) Inventor: Jason A. CARLYON, Richmond, VA (US)

(21) Appl. No.: 16/090,708

(22) PCT Filed: Mar. 29, 2017
(86) PCT No.: PCT/US2017/024669

§ 371 Date: Oct. 2, 2018

Related U.S. Application Data
(60) Provisional application No. 62/319,320, filed on Apr. 7, 2016.

(51) Int. Cl. A61K 39/02; A61K 39/40

(52) U.S. Cl. CPC A61K 39/0233; A61K 2039/552; A61K 39/40

(57) ABSTRACT

Anaplasma Marginale surface protein OmpA and homologous genes from Anaplasmatacaea family members are used in compositions suitable for vaccines to treat or prevent infections caused by tick-born bacteria of the Anaplasmatacaea family. OmpA proteins or peptide fragments may be used in combination with other Anaplasmatacaea surface proteins to elicit an immune response. Furthermore, antibodies to OmpA proteins can be used in diagnostic methods to determine whether an individual has contracted an Anaplasmatacaea infection.

### FIELD OF THE INVENTION

The invention generally relates to a vaccine and diagnostic for Anaplasmataceae infections. In particular, the invention provides A. marginale and A. phagocytophilum outer surface protein A (OmpA) epitopes.

### PRIORITY

This application claims priority to U.S. application 62/319,320 filed Apr. 7, 2016. This application is incorporated herein by reference in its entirety.

### SEQUENCE LISTING

This document incorporates by reference an electronic sequence listing text file, which was electronically submitted along with this document. The text file is named 02941112 ST25.txt, is 92 kilobytes, and was created on Mar. 22, 2017.

### BACKGROUND OF THE INVENTION

Anaplasma marginale is a Gram-negative obligate intracellular bacterium and the etiologic agent of bovine anaplasmosis, a debilitating infection that is transmitted biologically by ticks, mechanically via fly bites or blood-contaminated fomites, and vertically from mother to calf. It is a febrile illness, the symptoms of which can include anemia, weight loss, abortion, decreased milk production, and death. Due to these clinical manifestations, its propensity to become a chronic infection, and the costs associated with treatment, bovine anaplasmosis results in a combined economic loss for the United States and South American cattle industries that exceeds one billion dollars annually. In sub-Saharan Africa, where livestock sustain the livelihood of the rural poor, the disease can have devastating socioeconomic impacts. A. marginale is a member of the family Anaplasmataceae, which consists of veterinary and human obligate intracellular bacterial pathogens that reside within host cell derived vacuoles. A. marginale predominantly infects erythrocytes in vivo. Detection of the bacterium colocalizing with the endothelial cell marker, von Willebrand factor, in tissue sections from an experimentally inoculated calf indicate it is also capable of infecting endothelial cells in vivo and might serve as a reservoir for infection. Moreover, endothelial cell lines are useful for studying A. marginale infection in vitro, as they are the only mammalian cell type in which continuous cultivation of these microbes has been achieved. The immortalized tick cell line, ISE6, is susceptible to A. marginale infection and supports its replication, making it a useful model for studying bacterial-tick cell interactions.

The pathogen exhibits a biphasic developmental cycle in which it transitions between an infectious dense-cored (DC) form that mediates binding and entry and a non-infectious reticulate cell (RC) form that replicates by binary fission inside the A. marginale-occupied vacuole (AmV). Following replication, RCs reconvert to DCs that exit to invade naive host cells and thereby initiate new infections. Because A. marginale is an obligate intracellular bacterium, adhesins that mediate binding and entry into host cells are essential for survival. Such key virulence factors, however, are poorly defined.

A. marginale expresses the surface protein, OmpA (outer membrane protein A; AM854 in the St. Manes strain), during infection of cattle. OmpA is highly conserved among A. marginale sensu stricto strains and isolates, exhibiting 99.6 to 100% identity. Recent studies demonstrated the importance of OmpA proteins to cellular invasion by A. phagocytophilum (Aph) and Ehrlichia chaffeensis, two Anaplasmataceae members that cause potentially fatal infections of humans and animals. Indeed, it was discovered that A. phagocytophilum OmpA (ApOmpA) is one of a trio of adhesins that cooperatively function to mediate optimal bacterial binding to and invasion of host cells. However, the precise role of A. marginale OmpA (AmOmpA) in Anaplasmataceae infections has yet to be determined.

A. marginale subsp. centrale is used as live vaccine against bovine anaplasmosis in some parts of the world, but this results in unreliable protection as immunity is not uniform against all strains and outbreaks have occurred in immunized populations. Moreover, it is not USDA-approved, has a high production cost, and carries the risks of vaccine-induced disease and transmission of known and unknown pathogens.

Therefore, the need remains for compositions and methods to rapidly and accurately diagnosis new cases and to provide adequate vaccination against Anaplasmataceae infections that cause bovine anaplasmosis.

### SUMMARY

An aspect of the invention provides an immunogenic composition including one or more isolated polypeptides in a vehicle or carrier suitable for administration to a subject, wherein at least one of said one or more polypeptides consists of 5 to 19 consecutive residues of an Aph and A. marginale OmpA consensus binding region.

Another aspect of the invention provides a pharmaceutical composition comprising an antibody or an antigen binding fragment thereof and a pharmaceutically acceptable carrier, wherein said antibody or antigen binding fragment thereof specifically recognizes at least one epitope present in the Aph and A. marginale OmpA consensus binding region. In some embodiments, the antigen binding fragment is selected from the group consisting of Fab fragments, Fab′ fragments, F(ab′)₂ fragments, Fd fragments, Fv fragments, scFv fragments, and combinations thereof. In some embodiments, the antibody or antigen binding fragment thereof specifically recognizes at least one epitope consisting of 5 to 19 consecutive amino acids of the binding region including three residues found to be important for binding: A. marginale G55, K58, and K59.

Another aspect of the invention provides a method of protecting or treating a subject from a zoonotic disease comprising the step of administering to said subject an immunogenic or pharmaceutical composition as described herein. In some embodiments, the zoonotic disease is caused by an obligate intracellular Anaplasmataceae bacterium selected from the group consisting of Anaplasma phagocytophilum and Anaplasma marginale. In other embodiments, the subject is a cow and said zoonotic disease is bovine anaplasmosis.

Another aspect of the invention provides a method of determining if a subject has been exposed to or is infected with an obligate intracellular Anaplasmataceae bacterium selected from the group consisting of Anaplasma phagocytophilum and Anaplasma marginale, wherein said subject is suspected of having a zoonotic disease caused by an obligate intracellular Anaplasmataceae bacterium, comprising the steps of i) contacting a test sample from said subject, under conditions that allow polypeptide-antibody complexes to form, with a composition that includes one or more polypeptides, at least one of which consists of 5 to 19 consecutive residues of the Aph and A. marginale OmpA consensus binding region, ii) detecting one or more polypeptide-antibody complexes in said test sample, wherein the detection is an indication that antibodies specific for Anaplasmataceae OmpA are present in the test sample, and iii) determining said subject has been exposed to or is infected with said Anaplasmataceae bacterium if said antibodies specific for Anaplasmataceae OmpA are present in the test sample.

In some embodiment, the contacting and detecting steps are performed using an assay selected from the group consisting of an immunoblot and an enzyme-linked immunosorbent assay (ELISA). In some embodiments, the test sample is a body fluid selected from the group consisting of blood, plasma, serum, urine, and saliva.

### BRIEF DESCRIPTION OF THE DRAWINGS

FIG. 1A and B. Timeline of Aph infection cycle and differential transcription profiling of OMP candidate genes throughout the Aph infection cycle.

FIG. 1C-E. Differential transcription profiling of OMP candidate genes throughout the Aph infection cycle.

FIG. 2A-D. Differential expression analyses of ompA and asp14 during Aph invasion of HL-60 and RF/6A cells, during Aph binding to PSGL-1 CHO cells, and during transmission feeding of Aph infected I. scapularis ticks.

FIG. 3A-G. Aph expresses OmpA and Asp14 during infection of HL-60 cells and during murine and human infection.

FIGS. 4A and B. Trypsin treatment abolishes detection of Aph surface proteins and surface proteins Asp14 and OmpA are detected in Aph DC organisms.

FIG. 5A-D. Anti-OmpA does not disrupt bacterial cellular adherence or bacterial interaction with PSGL-1, but does partially neutralize Aph infection of HL-60 cells.

FIGS. 6A and B. Alignment of OmpA (SEQ ID NO:04) with Anaplasma and Ehrlichia species homologs AM854 (SEQ ID NO: 31), ACHIS 00486 (SEQ ID NO:33), ECH_0462 (SEQ ID NO:39), Ecaj_0563 (SEQ ID NO:45), and Erum 5620 (SEQ ID NO:51) with regions of identity and similarity shaded, and predicted 3D structure with extracellular loop and helix are indicated by arrows.

FIGS. 7A and B. Pretreatment of Aph with anti-OmpA reduces infection of HL-60 cells.

FIG. 8A-D. Model for how Aph OmpA interacts with its receptor to promote infection of host cells. A, Ap binding to sLex-capped PSGL-1 promotes entry; B, GST-OmpA binding to a 2,3-sialic acid of sLex blocks AP entry; C, Antibody binding to a 2,3-sialic acid of sLex blocks AP entry; and D, Antibody binding to PSGL-1 blocks Ap adhesion and entry.

FIG. 9A-D. Pretreatment of Aph with anti-Asp14 reduces infection of HL-60 cells.

FIG. 10A-D. Asp14 residues 101-124 are required to competitively inhibit Aph infection of mammalian host cells.

FIG. 11. An alignment of Asp14 residues 101-115, which constitute a conserved domain among homologs from Anaplasma and Ehrlichia species Asp14 (SEQ ID NO:01), AM936 (SEQ ID NO:13), ACHIS 00403 (SEQ ID NO:15), ECH_0377 (SEQ ID NO:19), Ecaj_0636 (SEQ ID NO:23), and Erum6320 (SEQ ID NO:27) with regions of identity and similarity shaded.

FIGS. 12A and B. Recombinant forms of Asp14 and OmpA cooperatively block Aph infection of HL-60 cells, either as full-length proteins or fragments identified as critical conserved effector domains.

FIG. 13A-C. Peptide antisera blocking reveals that the OmpA invasin domain lies within amino acids 59-74.

FIG. 14. Locations of linker insertion mutations that identify regions required to disrupt the ability of OmpA to antagonize Aph infection, showing alignment of OmpA (SEQ ID NO: 10) with Anaplasma and Ehrlichia species homologs AM854 (SEQ ID NO: 32), ACHIS_00486 (SEQ ID NO: 34), ECH_0462 (SEQ ID NO:40), Ecaj_0563 (SEQ ID NO:46), and Erum 5620 (SEQ ID NO:52) with regions of identity and similarity shaded.

FIG. 15. Percent of infection using linker insertion mutants of OmpA.

FIG. 16. Percent of infection in alanine substitution experiments that identified that OmpA aa59-74 are important for infection.

FIGS. 17A and B. ELISA results showing the specificity of antiserum raised against Asp14 aa98-112 or aa113-124.

FIG. 18. Percent of bacterial infection inhibited by pretreatment of Aph with anti-serum specific for Asp14 invasin domain.

FIG. 19. Percent of infection reduced by antisera specific for the OmpA invasin domain, Asp14 invasin domain, or combinations thereof.

FIGS. 20A and B. Western blot and ELISA showing that A. phagocytophilum OmpA and A. marginale OmpA share B-cell epitopes.

FIGS. 21A and B. AmOmpA and ApOmpA are structurally similar and exhibit conservation of glycine and lysine residues demonstrated to be important for adhesin function in ApOmpA. The predicted tertiary structures for ApOmpA and AmOmpA are highly similar. (A) Presented is a static image in which the predicted tertiary structures for ApOmpA and AmOmpA are overlaid to demonstrate their structural similarity. A PHYRE2 model of the mature sequence lacking signal peptide for each OmpA protein was generated, and the models were threaded onto each other using PyMol. (B) Zoom in of the image presented in panel A. Note that the alpha helices formed by the essential binding domain of ApOmpA and the putative AmOmpA binding domain overlap. ApOmpA functionally essential residues glycine 61 and lysine 64 correspond to AmOmpA G55 and K58.

FIG. 22A-D. Antibodies raised toward AmOmpA are specific. (A) Wells coated with GST alone, GST-AmOmpA, GST-ApOmpA, or AmOmpA50-67 were screened with antibodies targeting mature AmOmpA or AmOmpA50-67. Results shown are the mean±SD of triplicate samples. (B) GST-tagged ApOmpA and AmOmpA were subjected to Western blot analyses with anti-GST, anti-ApOmpA59-74, or anti-HisAmOmpA. (C) Western blot analyses of His-ApOmpA, His-AmOmpA, and His-OtOmpA using antibodies specific for the His tag, ApOmpA59-74, and AmOmpA50-67. (D) Rat anti-HisAmOmpA was used to screen Western-blotted A. marginale (Am) infected (I) and uninfected (U) RF/6A, ISE6 whole cell lysates, and A. phagocytophilum (Ap) infected and uninfected HL60 cell lysates.

FIG. 23A-H. Antisera raised against AmOmpA and AmOmpA50-67 inhibit infection. A. marginale DC organisms were incubated with preimmune serum, antiserum specific for mature AmOmpA, AmOmpA50-67 (A-D), or Fab fragments thereof (E-H) for 1 h followed by incubation with RF/6A cells in the continued presence of sera for 2 h. Unbound bacteria were removed and the infection was allowed to proceed for 48 h, after which the host cells were fixed and examined using immunofluorescence microscopy to determine the percentages of infected cells (A, C, E, and G) and the number of AmVs per cell (B, D, F, and H). Results are the means±SD of triplicate samples and are representative of three independent experiments with similar results. Statistically significant (* P<0.05; ** P<0.005; ***P<0.001) values are indicated.

FIGS. 24A and B. G61, K58, and K59 are critical for recombinant AmOmpA to optimally bind to mammalian host cells. RF/6A cells were incubated with His-tagged AmOmpA or versions thereof in which specific residues were substituted with alanine. The cells were successively incubated with His-tag antibody and Alexa Fluor 488-conjugated anti-mouse IgG and analyzed by flow cytometry. Representative histograms (A) and the mean fluorescence intensities±SD of triplicate samples (B) are presented. Data are representative of three independent experiments with similar results. Statistically significant (***P<0.001) values as compared to AmOmpA are indicated.

FIG. 25A-D. AmOmpA interacts with α2,3-sialic acid and α1,3-fucose on mammalian host cell surfaces. RF/6A cells were pretreated with α2,3/6-sialidase (A-B), a1,3/4-fucosidase (C-D), or vehicle control (A-D). Glycosidase and vehicle treated cells were incubated with His-AmOmpA (A-D), or media (cells or cells alone; A-D). The cells were fixed and screened using flow cytometry (A-D). Representative histograms showing His-AmOmpA binding to RF/6A cells are presented in panels A and C; mean fluorescence intensities±SD of triplicate samples are presented in panels B and D. Data shown are representative of three independent experiments with similar results. Statistically significant (***P<0.001) values are indicated.

FIGS. 26A and B. AmOmpA coated beads bind to and are internalized by endothelial cells. Fluorescent His-AmOmpA coated microspheres (AmOmpA beads) were incubated with RF/6A endothelial cells. (A) Binding was assessed by immunofluorescence microscopy after 1 h. (B) To assess internalization, cells were treated with trypsin after 8 h, washed, adhered to coverslips, fixed, and screened with an anti-His tag antibody by immunofluorescence microscopy. Results are the means±SD representative of three independent experiments done in triplicate with similar results. Statistically significant (** P<0.005; *** P<0.001) values are indicated.

FIG. 27A-D. Recombinant AmOmpA and ApOmpA competitively inhibit A. marginale infection of endothelial cells. RF/6A cells were incubated with GST alone, GST-AmOmpA (A and B), or GST-ApOmpA (C and D) proteins for 1 h. A. marginale DC organisms were then added and incubated with the cells in the presence of recombinant protein for 2 h. After washing to remove unbound bacteria, host cells were incubated for 48 h and subsequently examined by immunofluorescence microscopy to determine the percentage of infected cells (A and C) and AmVs per cell (B and D). Results are the means ±SD of triplicate samples and are representative of three independent experiments with similar results. Statistically significant (* P<0.05) values are indicated.

FIG. 28A-C. 6-sulfo sLex is dispensable for recombinant AmOmpA binding to RF/6A cell surfaces and for A. marginale infection. (A) RF/6A cells were incubated with CSLEX1, KM93, G72, or IgM control for 1 h followed by the addition of His-AmOmpA. Unbound recombinant protein was then washed away. Flow cytometry was used to detect bound His-AmOmpA. Cells alone served as a negative control. Histogram is representative of three independent experiments done in triplicate. (B and C) RF/6A endothelial cells were pretreated with IgM or G72. These cells were then incubated with DC A. marginale organisms for 2 h after which unbound bacteria were removed. Cells were examined after 48 h by immunofluorescence microscopy to determine the percentage of infected cells (B) and AmVs per cell (C).

FIG. 29A-H. AmOmpA contributes to A. marginale infection of tick cells. (A-D) Antisera raised against AmOmpA and AmOmpA₅₀₋₆₇ inhibit infection. A. marginale DC organisms were incubated with preimmune serum, antiserum specific for AmOmpA (A-B) or AmOmpA₅₀₋₆₇ (C-D) for 1 h followed by incubation with ISE6 cells in the continued presence of sera for 5 h. Unbound bacteria were removed and the infection was allowed to proceed for 72 h, after which the host cells were fixed and examined using immunofluorescence microscopy to determine the percentages of infected cells (A and C) and the number of AmVs per cell (B and D). (E-H) Recombinant AmOmpA and ApOmpA competitively inhibit A. marginale infection of tick cells. ISE6 cells were incubated with GST alone (E-H), GST-AmOmpA (E and F), or GST-ApOmpA (G and H) for 1 h. A. marginale DC organisms were then added and incubated with the cells in the presence of recombinant protein for 5 h. After washing to remove unbound bacteria, host cells were incubated for 72 h and subsequently examined by immunofluorescence microscopy to determine the percentage of infected cells (E and G) and AmVs per cell (F and H). Results are the means±SD of triplicate samples and are representative of three independent experiments with similar results. Statistically significant (* P<0.05; ** P<0.005) values are indicated.

### DETAILED DESCRIPTION

Aspects of the invention are related to diagnosing, preventing, and treating zoonotic diseases caused by Anaplasmataceae bacteria. The diseases affect both animals and humans and are collectively referred to as anaplasmosis, but more specifically known bovine anaplasmosis when transmitted to cows or HGA when transmitted to humans. The surface protein OmpA has been identified as mediating bacteria-host cell binding and entry. Thus, the surface protein OmpA and fragments thereof can be used for diagnosing whether a patient has been suffering from an Anaplasmataceae infection. Specifically, if antibodies to OmpA are identified in serum or other biological material from a subject suspected of an infection by suitable assay, such as ELISA or immunoblot, where, for example, the antibodies bind to or interact with OmpA proteins or fragments thereof, then it can be determined that the subject has been exposed to, infected with, or is currently infected with Anaplasmataceae bacteria. Furthermore, administration of OmpA proteins or fragments, or nucleic acids encoding for OmpA proteins, such as in forms where the nucleic acids are present with a vector such as a viral vector, or are present as purified peptides, polypeptides or proteins in a pharmaceutically acceptable carrier, can provide an immunogenic response in the subject and protection from subsequent infection, or provide for treatment by the production of antibodies to Anaplasmataceae infection in a subject that is already infected.

The critical regions of OmpA that mediate infection are highly conserved among family members A. phagocytophilum (Aph), A. marginale, and closely related Ehrlichia species, such as E. chaffeensis, E. canis, and E. ruminatium, and may be highly conserved in A. platys. In particular, Aph and A. marginale are closely related and express many gene homologs, including Asp14, OmpA and other surface antigens. The high degree of conservation makes these surface proteins ideal for producing a vaccine or immunogenic composition to provide protection from or therapy for multiple pathogens in humans and animals.

In one embodiment, the composition of the invention comprises one or more isolated and purified recombinant polypeptides. Each polypeptide comprises amino acid sequences encoding an OmpA invasin domain that mediates uptake of Anaplasmataceae bacteria into host cells. In some embodiments, the composition of the invention comprises the invasin domain of Aph OmpA, which lies within aa59-74 (SEQ ID NO:06: LKGPGKKVILELVEQL). This domain corresponds to aa53-68 of A. marginale OmpA (SEQ ID NO: 77: IKGSGKKVLLGLVERM). A consensus sequence is provided by SEQ ID NO: 78: X₁KGX₂GKKVX₃LX₄LVEX₅X₆, where X₁ is leucine or isoleucine, X₂ is proline or serine, X₃ is isoleucine or leucine, X₄ is glutamic acid or glycine, X₅ is glutamine or arginine, and X₆ is leucine or methionine.

In another embodiment, the composition comprises aa50-67 of A. Marginale OmpA (SEQ ID NO: 79: KYEIKGSGKKVLLGLVER) corresponding to aa56-73 of Aph Ompa (SEQ ID NO: 80: KYDLKGPGKKVILELVEQ). A consensus sequence is provided by SEQ ID NO: 81: KYX₁X₂KGX₃GKKVX₄LX₅LVEX₆, where X₁ is glutamic acid or aspartic acid, X₂ is leucine or isoleucine, X₃ is proline or serine, X₄ is isoleucine or leucine, X₅ is glutamic acid or glycine, and X₆ is glutamine or arginine.

In another embodiment, the composition comprises aa50-68 of A. Marginale OmpA (SEQ ID NO: 82: KYEIKGSGKKVLLGLVERM) corresponding to aa56-74 of Aph Ompa (SEQ ID NO: 83: KYDLKGPGKKVILELVEQL). A consensus sequence is provided by SEQ ID NO: 84: KYX₁X₂KGX₃GKKVX₄LX₅LVEX₆X₇, where X₁ is glutamic acid or aspartic acid, X₂ is leucine or isoleucine, X₃ is proline or serine, X₄ is isoleucine or leucine, X₅ is glutamic acid or glycine, X₆ is glutamine or arginine, and X₇ is methionine or leucine.

In some embodiments, the composition comprises or consists of at least 5 consecutive amino acids of SEQ ID NO: 84, e.g. 5, 6, 7, 8, 9, 10, 11, 12, 13, 14, 15, 16, 16, 18, or all 19 residues of SEQ ID NO: 84.

In other embodiments, a larger fragment of OmpA, e.g. encompassing all or a part of the full length Aph OmpA protein (SEQ ID NO:04) or the full length A. Marginale OmpA protein (SEQ ID NO: 31) is used. For example, a fragment such as aa21-79 of A. Marginale OmpA protein (SEQ ID NO: 32) is used. It is contemplated that virtually any protein sequence, as well as its corresponding nucleic acid sequence coding for the protein sequence that is or includes SEQ ID NO: 77 may be used. This would include the full length sequence as well as any sequence of, for example 5-50 (or less than 5 or more than 50) amino acids before the beginning or at the end of the amino acid sequence defined by of SEQ ID NO:77 or SEQ ID NO:78, and this can include amino acids which are present in the A. marginale OmpA full length sequence as well as amino acids which are from different species (e.g., a chimera) or from a synthetic sequence, e.g., a histidine or GST tag.

In one embodiment, the invention is a vaccine for prevention or treatment of anaplasmosis, such as bovine anaplasmosis. Administration of the composition of the invention stimulates an immune response in a subject and production of antibodies against OmpA. Because OmpA is on the outer surface of Anaplasmataceae bacteria, antibodies produced by the subject will block binding of bacteria to host cells and interfere with uptake into vacuoles. Bacteria unable to enter host cells will be detected by the host immune system and cleared from the body. Blockade can occur at the point of entry into neutrophils or endothelial cells or transfer between these two host cell types. Interruption of the zoonotic life cycle provides a further benefit to public health and well-being by breaking the chain of disease transmission to others.

In another embodiment, the invention directly provides antibodies for the prevention or treatment of anaplasmosis, such as bovine anaplasmosis. The antibodies recognize epitopes, e.g. within SEQ ID NO: 84 that are critical for binding to host cells. As described in Example 30, important residues for A. marginale binding include glycine at position 55 (G55), lysine at position 58 (K58), and lysine at position 59 (K59). Important residues for Aph binding include glycine at position 61 (G61) and lysine at position 64 (K64) which positionally align with A. marginale G55 and K58. Thus, an epitope of the invention may consist of 5 to 19 consecutive amino acids of SEQ ID NO: 84 including GX₃GKK (SEQ ID NO: 85) where X₃ is serine or proline. For example, the epitope may consist of 5, 6, 7, 8, 9, 10, 11, 12, 13, 14, 15, 16, 16, 18, or 19 consecutive amino acids of SEQ ID NO: 84 including SEQ ID NO: 85. GSGKK (SEQ ID NO:86) is provided when X₃ is serine and GPGKK (SEQ ID NO:87) is provided when X₃ is proline.

In another embodiment, the invention provides a method to detect the presence of OmpA in assays of biological samples obtained from subjects to bind to antibodies produced by an Anaplasmataceae-infected individual, either of which would be diagnostic for anaplasmosis. The preferred composition for diagnostic testing may comprise full length OmpA. However, compositions comprising fragments of OmpA or mixtures thereof are also contemplated. The assay used to detect antibodies may be any type of immunoassay, such as an immunoblot or an enzyme-linked immunosorbent assay. The test sample may be any type of body fluid, such as blood, plasma, serum, urine, saliva, or other body fluid. Tissues or cells may also be used, such as tissue sections or cell preparations adhered to slides or coverslips for immunohistochemical staining. The preferred embodiment is an ELISA with each protein type to independently detect antibodies to Asp14, and OmpA, however, a combination to detect Asp14 and OmpA antibodies in one ELISA is also contemplated.

In order to facilitate the understanding of the present invention, the following definitions are provided:
Aph: Anaplasma phagocytophilum or A. phagocytophilum, an Anaplasmataseae family bacterium that is tick-born and causes anaplasmosis in humans and animals.
Apl: Anaplasma platys or A. platys, an Anaplasmataseae family member bacterium that is tick-born and causes anaplasmosis that is restricted to dogs.
Anaplasmataceae: a family of closely related bacteria, including Anaplasma and Ehrlichia species. The genera Neorickettsia and Wolbachhia are also Anaplasmataceae, bacteria but do not cause anaplasmosis.
Antigen: term used historically to designate an entity that is bound by an antibody, and also to designate the entity that induces the production of the antibody. More current usage limits the meaning of antigen to that entity bound by an antibody, while the word “immunogen” is used for the entity that induces antibody production. Where an entity discussed herein is both immunogenic and antigenic, reference to it as either an immunogen or antigen will typically be made according to its intended utility. The terms “antigen”, “antigenic region” “immunogen” and “epitope” may be used interchangeably herein. As used herein, an antigen, immunogen or epitope is generally a portion of a protein (e.g. a peptide or polypeptide).
Asp14: 14-kilodalton Aph surface protein. OmpA homologs are expressed by Anaplasmataceae family members, including Aph, A. marginale, Ehrlichia chaffeensis, E. canis, E. ewingii, and E. ruminatium.
OmpA: Outer membrane protein A. OmpA homologs are expressed by Anaplasmataceae family members, including Aph, A. marginale, Ehrlichia chaffeensis, E. canis, E. ewingii, and E. ruminatium.
DC and RC: Aph undergoes a biphasic developmental cycle, the kinetics of which have been tracked in promyelocytic HL-60 cells. The cycle begins with attachment and entry of an infectious dense-cored (DC) organism. Once intracellular, the DC differentiates to the non-infectious reticulate cell (RC) form and replicates by binary fission to produce a bacteria-filled organelle called a morula. Later, the RCs transition back to DCs, which initiate the next round of infection.
Epitope: a specific chemical domain on an antigen that is recognized by a B-cell receptor, and which can be bound by secreted antibody. The term as used herein is interchangeable with “antigenic determinant”. An epitope may comprise a single, non-interrupted, contiguous chain of amino acids joined together by peptide bonds to form a peptide or polypeptide. Such an epitope can be described by its primary structure, i.e. the linear sequence of amino acids in the peptide chain. Epitope may also refer to conformational epitopes, which are comprised of at least some amino acids that are not part of an uninterrupted, linear sequence of amino acids, but which are brought into proximity to other residues in the epitope by secondary, tertiary and/or quaternary interactions of the protein. Residues in conformational epitopes may be located far from other resides in the epitope with respect to primary sequence, but may be spatially located near other residues in the conformational epitope due to protein folding.
Immunodominant epitope: The epitope on a molecule that induces the dominant, or most intense, immune response. The immunodominant epitope would elicit the greatest antibody titer during infection or immunization, as measured by, for example, the fraction of reactivity attributable to a certain antigen or epitope in an enzyme-linked immunosorbant assay as compared with the total responsiveness to an antigen set or entire protein.
Invasin domain: An invasin domain is a region of a pathogen's protein that binds a host cell and mediates intracellular signaling and pathogen entry into the host cell. In some cases, uptake of the pathogen results in the formation of a vacuole in which the intracellular pathogen will reside. The invasin domains of the invention are linear amino acid sequences within Asp14, OmpA, or other surface proteins that are found on the outer membrane of the bacteria Aph and other Anaplasmataceae family members, and can vary slightly from one family member to the next. However, the invasin domain in each Asp14 homolog is critical for uptake of bacteria into host cells (known to be neutrophils and endothelial cells in the case of Anaplasmataceae).
Linker sequences: short peptide sequences encoding functional units that may be engineered or otherwise added at the ends or within recombinant proteins, polypeptides, peptides of interest. Linker sequences may be used as “handles” for protein purification, as detectable signals of expression or binding to other proteins or macromolecules, to modulate tertiary structure, or enhance antigenicity. Examples of linker sequences include but are not limited to an amino acid spacer, an amino acid linker, a signal sequence, a stop transfer sequence, a transmembrane domain, and a protein purification ligand.
LINKER: a program to generate linker sequences for fusion proteins. Protein Engineering 13(5): 309-312, which is a reference that describes unstructured linkers. Structured (e.g. helical) sequence linkers may also be designed using, for example, existing sequences that are known to have that secondary structure, or using basic known biochemical principles to design the linkers.
Tags: Recombinant protein sequences that can be added to the N- or C-terminus of a recombinant protein for the purpose of identification or for purifying the recombinant protein for subsequent uses. Examples of recombinant protein tags that may be useful in practicing the invention include but are not limited to glutathione-S-transferease (GST), poly-histidine, maltose binding protein (MBP), FLAG, V5, halo, myc, hemaglutinin (HA), S-tag, calmodulin, tag, streptavidin binding protein (SBP), Softagl™, Softag3™, Xpress tag, isopeptag, Spy Tag, biotin carboxyl carrier protein (BCCP), GFP, Nus-tag, strep-tag, thioredoxin tag, TC tag, and Ty tag. All such tags are well-known to those of ordinary skill in the art of recombinant protein production.
Protein: Generally means a linear sequence of about 100 or more amino acids covalently joined by peptide bonds.
Polypeptide: Generally means a linear sequence of about 55 to about 100 amino acids covalently joined by peptide bonds.
Peptide: Generally means a linear sequence of about 55 or fewer amino acids covalently joined by peptide bonds.
Note: The terms “peptide”, “polypeptide” and “protein” may be used interchangeably herein.
Chimeric or fusion peptide or polypeptide: a recombinant or synthetic peptide or polypeptide whose primary sequence comprises two or more linear amino acid sequences which do not occur together in a single molecule in nature. The two or more sequences may be, for example, a peptide (e.g. an epitope or antigenic region) and a linker sequence, or two or more peptides (which may be the same or different) which are either contiguous or separated by a linker sequences, etc.
Tandem repeats: two or more copies of nucleic acid or amino acid sequences encoding the same peptide, which are arranged in a linear molecule and are either contiguous or separated by a linker sequences, etc.
Original or native or wild type sequence: The sequence of a peptide, polypeptide, protein or nucleic acid as found in nature.
Recombinant peptide, polypeptide, protein or nucleic acid: peptide, polypeptide, protein or nucleic acid that has been produced and/or manipulated using molecular biology techniques such as cloning, polymerase chain reaction (PCR), etc.
Synthetic peptide, polypeptide, protein or nucleic acid: peptide, polypeptide, protein or nucleic acid that has been produced using chemical synthesis procedures.
Type-specific: associated primarily with a single phyletic group.
Surface protein: A protein located on the outer surface membrane of a cell or bacterium.

TABLE 1
Aph Sequence Listing with SEQ ID Numbers.

| | | GENBANK | |
|---|---|---|---|
| | | ACCES- | |
| SEQ ID | PROTEIN | SION # | |
| NO | NAME | AND NAME | AMINO ACID SEQUENCE |
| SEQ ID | Full- | YP_504865 | MIPLAPWKSISVVYMSGSDEYKEIIK |
| NO: 01 | length | APH_0248 | QCIGSVKEVFGEGRFDDVVASIMKMQ |
| | Asp14 | | EKVLASSMQQDDTGTVGQIESGEGSG |
| | | | ARLSDEQVQQLMNSIREEFKDDLRAI |
| | | | KRRILKLERAVYGANTPKES |
| SEQ ID | Asp14 | APH_0248 | LRAIKRRILKLERAVYGANTPKES |
| NO: 02 | aa101- | | |
| | 124 | | |
| SEQ ID | Asp14 | APH_0248 | RAVYGANTPKES |
| NO: 03 | aa113- | | |
| | 124 | | |
| SEQ ID | Full | YP_504946 | MLRRSSFFCLLALLSVTSCGTLLPDS |
| NO: 04 | length | APH_0338 | NVGVGRHDLGSHRSVAFAKKVEKVYF |
| | OmpA | | DIGKYDLKGPGKKVILELVEQLRQDD |
| | | | SMYLVVIGHADATGTEEYSLALGEKR |
| | | | ANAVKQFIIGCDKSLAPRVTTQSRGK |
| | | | AEPEVLVYSTDAQEVEKANAQNRRA |
| | | | VIVVEFAHIPRSGVADMHAPVASSIT |
| | | | SENSNASAEGEDMEASEFSSAIAN |
| SEQ ID | OmpA | APH_0338 | CGTLLPDSNVGVGRHDLGSHRSVAFA |
| NO: 05 | aa19-74 | | KKVEKVYFDIGKYDLKGPGKKVILEL |
| | | | VEQLR |
| SEQ ID | OmpA | APH_0338 | LKGPGKKVILELVEQL |
| NO: 06 | aa59-74 | | |
| SEQ ID | OmpA | APH_0338 | EKVYFDIGK |
| NO: 07 | aa48-56 | | |
| SEQ ID | OmpA | APH_0338 | GHADATGTEEYSLALG |
| NO: 08 | | | |
| SEQ ID | OmpA | APH_0338 | LVYSTDAQEVEKANAQNRRAV |
| NO: 09 | | | |
| SEQ ID | OmpA | APH_0338 | PDSNVGVGRHDLGSHRSVAFAKKVE |
| NO: 10 | | | KVYFDIGKYDLKGPGKKVILELVEQL |
| | | | RQDDSMYLVVIGHADATGTEEYSLAL |
| | | | GEKRANAVKQFIIGCDKSLAPRVTTQ |
| | | | SRGKAEPEVLVYSTDAQEVEKANAQN |
| | | | RRAVIVVE |
| | | | FAHIPRSGVADM |
| SEQ ID | Asp14 | APH_0248 | LRAIKRRILKLE |
| NO: 11 | aa101- | | |
| | 112 | | |
| SEQ ID | Asp14 | APH_0248 | DEYKEIIKQCIGSVKEVFGEGRFDDV |
| NO: 12 | aa19-60 | | VASIMKMQEKVLASSM |

TABLE 2
Asp14 Homologs Sequence Listing with SEQ ID
Numbers

| | | | |
|---|---|---|---|
| SEQ ID | Anaplasma | AM936 | MSGEDEYKEIIRQCIGSVKEVFGEG |
| NO: 13 | marginale | | RFDDVVASIMKMQEKVLASSMKDGD |
| | | | PVGQIAADGVGNELYDRIADRLEER |
| | | | VSQKISEDLRIIKKRLLRLERVVLG |
| | | | GGSVSGDAAAHQVSGNQPSQQNSSA |
| | | | AAEGG |
| SEQ ID | A. | AM936 | LGGGSVSGDAAAHQVSGNQPSQQNS |
| NO: 14 | marginale | | SAAAEGG |
| SEQ ID | A. | ACIS_ | MSGEDEYKEIIRQCIGSVKEVFGEG |
| NO: 15 | marginale | 00403 | RFDDVVASIMKMQEKVLASSMKDGD |
| | subspecies | | PVGQIAADGVGNELYDRIADRLEER |
| | Centrale | | VSQKISEDLRIIKKRLLRLERVVLG |
| | | | GGSVSGDAAAAHQVSGNQPSQQNSS |
| | | | AAAEGG |
| SEQ ID | A. | ACIS_ | LGGGSVSGDAAAAHQVSGNQPSQQN |
| NO: 16 | marginale | 00403 | SSAAAEGG |
| | subspecies | | |
| | Centrale | | |
| SEQ ID | A. | AM936 & | MSGEDEYKEIIRQCIGSVKEVFGEG |
| NO: 17 | marginale & | ACIS- | RFDDVVASIMKMQEKVLASSM |
| | A. | 00403 | |
| | marginale | | |
| | subspecies | | |
| | Centrale | | |
| SEQ ID | A. | AM936 & | DLRIIKKRLLRLERVV |
| NO: 18 | marginale & | ACIS- | |
| | A. | 00403 | |
| | marginale | | |
| | subspecies | | |
| | Centrale | | |
| SEQ ID | Ehrlichia | ECH_0377 | MAEDDYKGVIKQYIDTVKEIVGDSK |
| NO: 19 | chaffeensis | | TFDQMFESVVRIQERVMAANAQNNE |
| | | | DGVIDNGDQVKRIGSSTSESISNTE |
| | | | YKELMEELKVIKKRILRLERKILKP |
| | | | KEEV |
| SEQ ID | E. | ECH_0377 | MAEDDYKGVIKQYIDTVKEIVGDSK |
| NO: 20 | chaffeensis | | TFDQMFESVVRIQERVM |
| SEQ ID | E. | ECH_0377 | ELKVIKKRILRLE |
| NO: 21 | chaffeensis | | |
| SEQ ID | E. | ECH_0377 | RKILKPKEEV |
| NO: 22 | chaffeensis | | |
| SEQ ID | E. canis | Ecaj_ | MADDEYKGVIQQYINTVKEIVSDSK |
| NO: 23 | | 0636 | TFDQMFESVVKIQERVMEANAQNDD |
| | | | GSQVKRIGSSTSDSISDSQYKELIE |
| | | | ELKVIKKRLLRLEHKVLKPKEGA |
| SEQ ID | E. canis | Ecaj_ | MADDEYKGVIQQYINTVKEIVSDSK |
| NO: 24 | | 0636 | TFDQMFESVVKIQERVM |
| SEQ ID | E. canis | Ecaj_ | ELKVIKKRLLRLE |
| NO: 25 | | 0636 | |
| SEQ ID | E. canis | Ecaj_ | HKVLKPKEGA |
| NO: 26 | | 0636 | |
| SEQ ID | E. | Erum6320 | MADEDYKGVIKQYIDTVKEIVGDSK |
| NO: 27 | ruminantium | | TFDQMFESVVKIQERVMAASAQNEA |
| | | | NGALVEGDSKMKRIRSADDSIAYTQ |
| | | | SQELLEELKVLKKRIARLERHVFKS |
| | | | NKTEA |
| SEQ ID | E. | Erum6320 | MADEDYKGVIKQYIDTVKEIVGDSK |
| NO: 28 | ruminantium | | TFDQMFESVVKIQERVM |
| SEQ ID | E. | Erum6320 | ELKVLKKRIARLE |
| NO: 29 | ruminantium | | |
| SEQ ID | E. | Erum6320 | RHVFKSNKTEA |
| NO: 30 | ruminantium | | |

TABLE 3
OmpA Homologs Sequence Listing with SEQ ID
Numbers

| | | | |
|---|---|---|---|
| SEQ ID | Anaplasma | AM854 | MLHRWLALCFLASFAVTGCGLF |
| NO: 31 | marginale | | SKEKVGMDIVGVPFSAGRVEKV |
| | | | YFDFNKYEIKGSGKKVLLGLVE |
| | | | RMKADKRSTLLIIGHTDSRGTE |
| | | | EYNLALGERRANAVKEFILGCD |
| | | | RSLSPRISTQSRGKAEPEVLVY |
| | | | SSDFKEAEKAHAQNRRVVLIVE |
| | | | CQHSVSPKKKMAIKWPFSFGRS |
| | | | AAKQDDVGSSEVSDENPVDDSS |
| | | | EGIASEEAAPEEGVVSEEAAEE |
| | | | APEVAQDSSAGVVAPE |
| SEQ ID | A. | AM854 | LFSKEKVGMDIVGVPFSAGRVE |
| NO: 32 | marginale | | KVYFDFNKYEIKGSGKKVLLGL |
| | | | VERMKADKRSTLLII |
| SEQ ID | A. | ACIS_ | MLHRWLALCLLASLAVTGCELF |
| NO: 33 | marginale | 00486 | NKEKVNIDIGGVPLSAGRVEKV |
| | subspecies | | YFDFNKYEIKGSGKKVLLGLVE |
| | Centrale | | RMKADKMSTLLIVGHTDSRGTE |
| | | | EYNLALGERRANAVKEFILGCD |
| | | | RSLSPRISTQSRGKAEPEILVY |
| | | | SSDFKEAEKAHAQNRRVVLIME |
| | | | CQHAASPKKARVSRWPFSFGRS |
| | | | SATQQDNGGGTVAAGSPGEDAP |
| | | | AEVVEPEETQEAGE |
| SEQ ID | A. | ACIS_ | LFNKEKVNIDIGGVPLSAGRVE |
| NO: 34 | marginale | 00486 | KVYFDFNKYEIKGSGKKVLLGL |
| | subspecies | | VERMKADKMSTLLIV |
| | Centrale | | |
| SEQ ID | A. | AM854 & | AGRVEKVYFDFNKYEIKGSGKK |
| NO: 35 | marginale & | ACIS- | VLLGLVERMKAD |
| | A. | 00486 | |
| | marginale | | |
| | subspecies | | |
| | Centrale | | |
| SEQ ID | A. | AM936 & | GHTDSRGTEEYNLALG |
| NO: 36 | marginale & | ACIS- | |
| | A. | 00403 | |
| | marginale | | |
| | subspecies | | |
| | Centrale | | |
| SEQ ID | A. | AM854 & | RRANAVKEFILGCDRSLSPRIS |
| NO: 37 | marginale & | ACIS- | TQSRGKAE |
| | A. | 00486 | |
| | marginale | | |
| | subspecies | | |
| | Centrale | | |
| SEQ ID | A. | AM854 & | LVYSSDFKEAEKAHAQNRRVVL |
| NO: 38 | marginale & | ACIS- | I |
| | A. | 00486 | |
| | marginale | | |
| | subspecies | | |
| | Centrale | | |
| SEQ ID | Ehrlichia | ECH_ | MKHKLVFIKFMLLCLILSSCKT |
| NO: 39 | chaffeensis | 0462 | TDHVPLVNVDHVFSNTKTIEKI |
| | | | YFGFGKATIEDSDKTILEKVMQ |
| | | | KAEEYPDTNIIIVGHTDTRGTD |
| | | | EYNLELGKQRANAVKDFILERN |
| | | | KSLEDRIIIESKGKSEPAVLVY |
| | | | SNNPEEAEYAHTKNRRVVITLT |
| | | | DNLIYKAKSSDKDPSSNKTEQ |
| SEQ ID | Ehrlichia | ECH_ | NVDHVFSNTKTIEKIYFGFGKA |
| NO: 40 | chaffeensis | 0462 | TIEDSDKTILEKVMQKAEEYPD |
| | | | TNIIIV |
| SEQ ID | Ehrlichia | ECH_ | IEDSDKTILEKVMQKAEEYPDT |
| NO: 41 | chaffeensis | 0462 | NIIIV |
| SEQ ID | Ehrlichia | ECH_ | GHTDTRGTDEYNLELGE |
| NO: 42 | chaffeensis | 0462 | |
| SEQ ID | Ehrlichia | ECH_ | QRANAVKDFILERNKSLEDRII |
| NO: 43 | chaffeensis | 0462 | IESKGKSEPAV |
| SEQ ID | Ehrlichia | ECH_ | LVYSNNPEEAEYAHTKNRRVVI |
| NO: 44 | chaffeensis | 0462 | |
| SEQ ID | E. canis | Ecaj_ | MKHKLVFIKFILLCLILSSCKT |
| NO: 45 | | 0563 | TDHVPLVNTDHVFSNMKTIEKI |
| | | | YFDFGKATIGDSDKAILEKVIQ |
| | | | KAQKDTNTNIVIVGHTDTRGTD |
| | | | EYNLELGEQRANAVKDFIIEHD |
| | | | KSLENRITVQSKGKSEPAVLVY |
| | | | SSNPEEAEHAHAKNRRVVITLT |
| | | | DNGNKTSQ |
| SEQ ID | E. canis | Ecaj_ | TTDHVPLVNTDHVFSNMKTIEK |
| NO: 46 | | 0563 | IYFDFGKATIGDSDKAILEKVI |
| | | | QKAQKDTNTNIVIV |
| SEQ ID | E. canis | Ecaj_ | GDSDKAILEKVIQKAQKDTNTN |
| NO: 47 | | 0563 | IVIV |
| SEQ ID | E. canis | Ecaj_ | GHTDTRGTDEYNLELGE |
| NO: 48 | | 0563 | |
| SEQ ID | E. canis | Ecaj_ | QRANAVKDFIIEHDKSLENRIT |
| NO: 49 | | 0563 | VQSKGKSEPAV |
| SEQ ID | E. canis | Ecaj_ | LVYSSNPEEAEHAHAKNRRVVI |
| NO: 50 | | 0563 | |
| SEQ ID | E. | Erum5620 | MRYQLIVANLILLCLTLNGCHF |
| NO: 51 | ruminantium | | NSKHVPLVNVHNLFSNIKAIDK |
| | | | VYFDLDKTVIKDSDKVLLEKLV |
| | | | QKAQEDPTTDIIIVGHTDTRGT |
| | | | DEYNLALGEQRANAVRDFIISC |
| | | | DKSLEKRITVRSKGKSEPAILV |
| | | | YSNNPKEAEDAHAKNRRVVITL |
| | | | VNNSTSTDNKVPTTTTPFNEEA |
| | | | HNTISKDQENNTQQQAKSDNIN |
| | | | NINTQQKLEQDNNNTPEVN |
| SEQ ID | E. | Erum5620 | NSKHVPLVNVHNLFSNIKAIDK |
| NO: 52 | ruminantium | | VYFDLDKTVIKDSDKVLLEKLV |
| | | | QKAQEDPTTDIIIV |
| SEQ ID | E. | Erum5620 | DSDKVLLEKLVQKAQEDPTTDI |
| NO: 53 | ruminantium | | IIV |
| SEQ ID | E. | Erum5620 | GHTDTRGTDEYNLALGE |
| NO: 54 | ruminantium | | |
| SEQ ID | E. | Erum5620 | QRANAVRDFIISCDKSLEKRIT |
| NO: 55 | ruminantium | | VRSKGKSEPAI |
| SEQ ID | E. | Erum5620 | LVYSNNPKEAEDAHAKNRRVVI |
| NO: 56 | ruminantium | | |

In addition to sequences for Aph OmpA and Asp14 shown in Table 1, and homologs shown in Tables 2-3, other surface proteins that Aph preferentially expresses in human versus tick cells may be used. Table 4 shows examples of proteins that can be included in the “cocktail” of peptides, polypeptides or protein sequences of the composition of the invention. Examples of these include APH_0915, APH_1325 (Msp2), APH_1378, APH_1412, APH_0346, APH_0838, APH_0839, APH_0874, and APH_0906 because all are upregulated 3- to 60-fold during RC-DC transition, DC exit, and/or reinfection and our surface proteomic study indicates that they are surface proteins. The file names for each of the aforementioned proteins are from the A. phagocytophilum HZ annotated genome. A similar expression profile is exhibited by APH_1235, which is another late stage gene that is upregulated 70-fold, as taught by Mastronunzio and colleagues, who identified APH_1235 as an A. phagocytophilum surface protein. P44 is a 44 kilodalton surface protein and is the bacterium's major surface protein. Synonyms of P44 are Msp2 (major surface protein 2) and Msp2 (P44). All Anaplasma species encode P44 proteins and there are huge repertoires of P44 genes in these bacterial species' chromosomes. For instance, the annotated Aph strain HZ genome encode 113 P44 proteins. These exist as complete genes or pseudogenes (incomplete genes). There is one expression site for p44 genes. Basically, different p44 genes get shuffled into the expression site by a process known as gene conversion with the end result being that Aph (and other Anaplasma species) can vary the P44 protein on their cell surfaces, a process called antigenic variation. This enables them to perpetually evade the humoral immune response.

TABLE 4
Anaplamatacaea Surface Proteins Sequence Listing and SEQ ID Numbers

| | | |
|---|---|---|
| SEQ ID NO: 57 | Full-length APH_0915 | Genbank Accession No: YP_505488 |
| SEQ ID NO: 58 | Full-length APH_1378 | Genbank Accession No: YP_505877 |
| SEQ ID NO: 59 | Full-length APH_1412 | Genbank Accession No: YP_505903 |
| SEQ ID NO: 60 | Full-length APH_0346 | Genbank Accession No: YP_504953 |
| SEQ ID NO: 61 | Full-length APH_0838 | Genbank Accession No: YP_505415 |
| SEQ ID NO: 62 | Full-length APH_0839 | Genbank Accession No: YP_505416 |
| SEQ ID NO: 63 | Full-length APH_0874 | Genbank Accession No: YP_505450 |
| SEQ ID NO: 64 | Full-length APH_0906 | Genbank Accession No: YP_505479 |
| SEQ ID NO: 65 | Full-length APH_1325 | Genbank Accession No: YP_505833 |
| | (Msp2) | |
| SEQ ID NO: 66 | Full-length APH_1235 | Genbank Accession No: YP_505764 |

In addition to polypeptides sequences from Aph surface proteins, other sequences may be included in the polypeptides of the invention. Such sequences include but are not limited to antigenic peptide sequences such as linker sequences which in and of themselves are antigenic. Examples of recombinant protein tags that may be useful in practicing the invention include but are not limited to glutathione-S-transferease (GST), poly-histidine, maltose binding protein (MBP), FLAG, V5, halo, myc, hemaglutinin (HA), S-tag, calmodulin, tag, streptavidin binding protein (SBP), Softag1™, Softag3™, Xpress tag, isopeptag, Spy Tag, biotin carboxyl carrier protein (BCCP), GFP, Nus-tag, strep-tag, thioredoxin tag, TC tag, and Ty tag. Examples of linker sequences include but are not limited to an amino acid spacer, an amino acid linker, a signal sequence, a stop transfer sequence, a transmembrane domain, and a protein purification ligand. It should also be recognized that a multitude of other such sequences are known to those of skill in the art, and inclusion of other antigenic, linker, or tag sequences is contemplated.

Those of skill in the art will recognize that, while in some embodiments of the invention, the amino acid sequences that are chosen for inclusion in the polypeptides of the invention correspond exactly to the primary amino acid sequence of the original or native sequences of an Asp14 or OmpA protein, this need not always be the case. The amino acid sequence of an epitope that is included in the polypeptides of the invention may be altered somewhat and still be suitable for use in the present invention. For example, certain conservative amino acid substitutions may be made without having a deleterious effect on the ability of the polypeptides to elicit an immune response. Those of skill in the art will recognize the nature of such conservative substitutions, for example, substitution of a positively charged amino acid for another positively charged amino acid (e.g. K for R or vice versa); substitution of a negatively charged amino acid for another negatively charged amino acid (e.g. D for E or vice versa); substitution of a hydrophobic amino acid for another hydrophobic amino acid (e.g. substitution of A, V, L, I, W, etc. for one another); etc. All such substitutions or alterations of the sequences of the polypeptides that are disclosed herein are intended to be encompassed by the present invention, so long as the resulting polypeptides still function to elicit a suitable immune response. In addition, the amino acid sequences that are included in the polypeptides or any chimeric proteins of the invention need not encompass a full length native polypeptide. Those of skill in the art will recognize that truncated versions of amino acid sequences that are known to be or to contain antigenic polypeptides may, for a variety of reasons, be preferable for use in the practice of the invention, so long as the criteria set forth for an epitope is fulfilled by the sequence. Amino acid sequences that are so substituted or otherwise altered may be referred to herein as “based on” or “derived from” the original wild type or native sequence. In general, the OmpA proteins or polypeptide fragments from which the linear epitopes are “derived” or on which the linear epitopes are “based” are the OmpA proteins or peptide fragments as they occur in nature. These natural OmpA proteins may alternatively be referred to as native or wild type proteins.

Such changes to the primary sequence may be introduced for any of a variety of reasons, for example, to eliminate or introduce a protease cleavage site, to increase or decrease solubility, to promote or discourage intra- or inter-molecular interactions such as folding, ionic interactions, salt bridges, etc, which might otherwise interfere with the presentation and accessibility of the individual epitopes along the length of a peptide or polypeptide. All such changes are intended to be encompassed by the present invention, so long as the resulting amino acid sequence functions to elicit a protective antibody response in a host to whom it is administered. In general, such substituted sequences will be at least about 50% identical to the corresponding sequence in the native protein, preferably about 60 to 70, or even 70 to 80, or 80 to 90% identical to the wild type sequence, and preferably about 95, 96, 97, 98, 99, or even 100% identical to a native OmpA sequence or peptide fragment. The reference native OmpA sequence or peptide fragment may be from any suitable type of Anaplasmataceae, e.g. from any Anaplasmataceae which is known to infect mammals.

In some embodiments of the invention, individual linear epitopes in a chimeric vaccinogen are separated from one another by intervening sequences that are more or less neutral in character, i.e. they do not in and of themselves elicit an immune response to Anaplasmataceae. Such sequences may or may not be present between the epitopes of a chimera. If present, they may, for example, serve to separate the epitopes and contribute to the steric isolation of the epitopes from each other. Alternatively, such sequences may be simply artifacts of recombinant processing procedures, e.g. cloning procedures. Such sequences are typically known as linker or spacer peptides, many examples of which are known to those of skill in the art. See, for example, Crasto, C. J. and J. A. Feng. 2000.

In addition, other elements may be present in chimeric proteins, for example leader sequences or sequences that “tag” the protein to facilitate purification or detection of the protein, examples of which include but are not limited to tags that facilitate detection or purification (e.g. S-tag, or Flag-tag), other antigenic amino acid sequences such as known T-cell epitope containing sequences and protein stabilizing motifs, etc. In addition, the chimeric proteins may be chemically modified, e.g. by amidation, sulfonylation, lipidation, or other techniques that are known to those of skill in the art.

The invention further provides nucleic acid sequences that encode chimeric proteins of the invention. Such nucleic acids include DNA, RNA, and hybrids thereof, and the like. Further, the invention comprehends vectors which contain or house such coding sequences. Examples of suitable vectors include but are not limited to plasmids, cosmids, viral based vectors, expression vectors, etc. In a preferred embodiment, the vector will be a plasmid expression vector.

The chimeric proteins of the invention may be produced by any suitable method, many of which are known to those of skill in the art. For example, they may be chemically synthesized, or produced using recombinant DNA technology (e.g. in bacterial cells, in cell culture (mammalian, yeast or insect cells), in plants or plant cells, or by cell-free prokaryotic or eukaryotic-based expression systems, by other in vitro systems, etc.). In some embodiments, the polypeptides are produced using chemical synthesis methods.

The present invention also provides compositions for use in eliciting an immune response. The compositions may be utilized as vaccines to prevent or treat anaplasmosis, particularly when manifested in cows as bovine anaplasmosis. By eliciting an immune response, we mean that administration of the antigen causes the synthesis of specific antibodies (at a titer as described above) and/or cellular proliferation, as measured, e.g. by ³H thymidine incorporation, or by other known techniques. By “vaccine” we mean a linear polypeptide, a mixture of linear polypeptides or a chimeric or fusion polypeptide that elicits an immune response, which results in protection of an organism against challenge with an Anaplasmataceae species bacterium. The protective response either wholly or partially prevents or arrests the development of symptoms related to anaplasmosis, in comparison to a non-vaccinated (e.g. adjunct alone) control organisms, in which disease progression is not prevented. The compositions include one or more isolated and substantially purified polypeptides or chimeric peptides as described herein, and a pharmacologically suitable carrier. The polypeptides or chimeric peptides in the composition may be the same or different, i.e. the composition may be a “cocktail” of different polypeptides or chimeric peptides, or a composition containing only a single type of polypeptide or chimeric peptide. The preparation of such compositions for use as vaccines is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients or carriers are, for example, water, saline, dextrose, glycerol, ethanol and the like, or combinations thereof. In addition, the composition may contain minor amounts of auxiliary substances such as wetting or emulsifying agents, pH buffering agents, and the like. The vaccine preparations of the present invention may further comprise an adjuvant, suitable examples of which include but are not limited to Seppic, Quil A, Alhydrogel, etc. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of polypeptides or chimeric peptides in the formulations may vary. However, in general, the amount in the formulations will be from about 0.01-99%, weight/volume.

The methods involve administering a composition comprising recombinant polypeptides or chimeric peptides in a pharmacologically acceptable carrier to a mammal. The mammal may be a cow, but this need not always be the case. Because anaplasmosis is a zoonotic disease that causes anaplasmosis in all known mammalian hosts, human and veterinary applications of this technology are also contemplated. The vaccine preparations of the present invention may be administered by any of the many suitable means which are well known to those of skill in the art, including but not limited to by injection, inhalation, orally, intranasally, by ingestion of a food product containing the polypeptides or chimeric peptides, etc. In some embodiments, the mode of administration is subcutaneous or intramuscular. In addition, the compositions may be administered in conjunction with other treatment modalities such as substances that boost the immune system, various anti-bacterial chemotherapeutic agents, antibiotics, and the like.

The present invention provides methods to elicit an immune response to Anaplasmataceae and/or to vaccinate against Anaplasmataceae infection in mammals. In one embodiment, the mammal is a cow. In another embodiment, the mammal is a human. Those of skill in the art will recognize that other mammals exist for which such vaccinations would also be desirable, e.g. the preparations may also be used for veterinary purposes. Examples include but are not limited to companion “pets” such as dogs, cats, etc.; food source, work and recreational animals such as cattle, horses, oxen, sheep, pigs, goats, and the like; or even wild animals that serve as a reservoir of Anaplasmataceae, particularly wild animals adapted to living in close proximity to urban areas (e.g. mice, deer, rats, raccoons, opossum, coyotes, etc).

The invention also provides a diagnostic and a method for using the diagnostic to identify subjects who have antibodies to the epitopes contained within the polypeptides or chimeric proteins of the invention. A biological sample from an individual (e.g. a cow, a deer, or other mammals susceptible to infection by Anaplasmataceae) suspected of having been exposed to Anaplasmataceae, or at risk for being exposed to Anaplasmataceae, is contacted with the peptides, polypeptides, or chimeric proteins of the invention. Using known methodology, the presence or absence of a binding reaction between the polypeptides or chimeric proteins and antibodies in the biological sample is detected. A positive result (i.e. binding occurs, thus antibodies are present) indicates that the individual has been exposed to and/or is infected with Anaplasmataceae. Further, the diagnostic aspects of the invention are not confined to clinical use or home use, but may also be valuable for use in the laboratory as a research tool, e.g. to identify Anaplasmataceae bacteria isolated from ticks, to investigate the geographical distribution of Anaplasmataceae species and strains, etc.

The present invention also encompasses antibodies to the epitopes and/or to the polypeptides or chimeric proteins disclosed herein. Such antibodies may be polyclonal, monoclonal or chimeric, and may be generated in any manner known to those of skill in the art. In a preferred embodiment of the invention, the antibodies are bactericidal, i.e. exposure of Anaplasmataceae bacteria to the antibodies causes death of the bacteria. Such antibodies may be used in a variety of ways, e.g. as detection reagents to diagnose prior exposure to Anaplasmataceae, as a reagent in a kit for the investigation of Anaplasmataceae, to treat Anaplasmataceae infections, etc.

Alternatively, appropriate antigen fragments or antigenic sequences or epitopes may be identified by their ability, when included in polypeptides or chimeric proteins, to elicit suitable antibody production to the epitope in a host to which the polypeptides or chimeric proteins are administered. Those of skill in the art will recognize that definitions of antibody titer may vary. Herein, “titer” is taken to be the inverse dilution of antiserum that will bind one half of the available binding sites on an ELISA well coated with 100 ng of test protein. In general, suitable antibody production is characterized by an antibody titer in the range of from about 100 to about 100,000, and preferably in the range of from about 10,000 to about 10,000,000. Alternatively, and particularly in diagnostic assays, the “titer” should be about three times the background level of binding. For example, to be considered “positive”, reactivity in a test should be at least three times greater than reactivity detected in serum from uninfected individuals. Preferably, the antibody response is protective, i.e. prevents or lessens the development of symptoms of disease in a vaccinated host that is later exposed to Anaplasmataceae, compared to an unvaccinated host.

The following Examples are provided to illustrate various embodiments of the invention, however, as described in detail above, aspects of the invention can be practiced in a variety of ways different from those illustrated in the Examples.

### EXAMPLES

The following experimental procedures were used in the examples of the invention:

Cell lines and cultivation of uninfected and Aph-infected HL-60 cells. PSGL-1 CHO cells and RF/6A cells were cultivated as described [21,77]. Uninfected HL-60 cells (American Type Culture Collection [ATCC]; Manassas, VA; ATCC code CCL-240) and HL-60 cells infected with the Aph NCH-1 strain or a transgenic HGE1 strain expressing GFP (a gift from Ulrike Munderloh of the University of Minnesota, Minneapolis, MN) were cultivated. Spectinomycin (Sigma-aldrich, St. Louis, Mo.) was added to HL-60 cultures harboring transgenic HGE1 bacteria at a final concentration of 100 μg/ml.

Aph DC organism surface biotinylation and affinity purification. Aph DC organisms from 10⁹ infected (≥90%) HL-60 cells were enriched for by sonication followed by differential centrifugation as described [61]. To purify DC organisms away from the majority of contaminating host and RC organism cellular debris, the sonicate was fractionated using discontinuous Renografin (diatrizoate sodium, Bracco diagnostics, Princeton, N.J.) density gradient centrifugation. Purified DC organisms were resuspended in 1 ml of phosphate-buffered saline (PBS) (pH 8.0) containing 1 mM MgCl₂ and 10 mM Sulfo-NHS-SS-Biotin (Pierce; Rockland, Ill.) and incubated for 30 min at room temperature. Free biotin was quenched by washing the sample with 50 mM Tris (pH 8.0), followed by two washes with PBS. Biotinylated bacteria were solubilized in radioimmunoprecipitation assay (RIPA) buffer (25 mM Tris-HCl [pH 7.6], 150 mM NaCl, 1% NP-40, 1% sodium deoxycholate, 0.1% sodium dodecyl sulfate [SDS], 1 mM sodium orthovanadate, 1 mM sodium fluoride, and Complete EDTA-free protease inhibitor set cocktail [Roche, Indianapolis, Ind.]) on ice for 1 h. Every 20 min during the 1-h incubation, the sample was subjected to eight 8-s bursts on ice interspersed with 8-s rest periods using a Misonix S4000 ultrasonic processor (Farmingdale, N.Y.) on an amplitude setting of 30. Insoluble material was removed by spinning at 10,000×g for 10 min at 4° C. To purify biotinylated proteins, the clarified lysate was mixed with High Capacity NeutrAvidin agarose beads (Pierce) by end-over-end rotation overnight at 4° C. The gel slurry was pelleted by centrifugation at 1,000×g for 1 min. After removal of the supernatant, the beads were resuspended in eight ml PBS and parceled into ten 800 μl aliquots, each of which were added to spin columns optimized for affinity purification (Pierce). The columns were washed three times with PBS and centrifuged at 1,000×g to remove any non-biotinylated proteins. The captured biotinylated proteins were eluted from the beads by end-over-end rotation with 150 mM DTT in 0.25% sodium deoxycholate for 2 h at room temperature. The agarose beads were centrifuged at 1,000×g for 2 min and the supernatant containing the biotinylated proteins was saved. The Bradford assay was used to determine the protein concentration of the eluate. The majority of the sample was stored at 4° C. until analysis. To ensure that this procedure had enriched for DC bacterial surface proteins, an aliquot of the affinity-purified sample was resolved by SDS-PAGE alongside an Aph whole-cell lysate, neutravidin beads plus unlabeled DC whole cell lysate, and neutravidin beads alone followed by silver staining.

2D-LC/MS-MS proteome analysis. Unless otherwise stated, all buffers were made with LC/MS grade solvents (Fisher Chemical, Fairlawn, N.J.). Samples were processed for proteomic analysis as described previously with the following methodological details. Following biotinylation enrichment of Aph surface proteins, 300 μg of protein mass in 400 μl of lysis buffer was concentrated and exchanged into 25 μl of ammonium bicarbonate buffer (ABC) (50 mM NH₄CO₃/0.05% C₂₄H₃₉O₄Na) using a Centriprep YM-10 filter unit (Millipore, Billerica, Mass.). DTT was added to achieve a final concentration of 20 mM, and disulfide bonds were reduced at 90° C. for 30 min. After cooling to room temperature, cysteine alkylation was performed on the sample with freshly prepared iodoacetamide (32 mM) for 30 min at room temperature in the dark. Trypsin Gold (100 ng/μl; Promega, Madison, Wis.) was added to a final 1:100 enzyme:protein ratio, and the sample was incubated at 37° C. overnight. The digested sample was dried within a speed vacuum and stored dry at −20° C.

The digest sample was reconstituted in 60 μL of 100 mM ammonium formate (pH 10) for multidimensional peptide separation and mass spectrometry analysis on a 2D-nanoAcquity chromatography system online with a Synapt quadrupole/time-of-flight tandem mass spectrometer (Waters) as previously reported. Two-replicate injections were analyzed for the sample. Resulting data were processed using PLGS software, v2.4 (Waters) as described elsewhere. Data were then search against an Aph-specific FASTA database (RefSeq and Uniprot sources; downloaded Feb. 2010) and its reversed-sequences as a decoy database. Search parameters required a minimum precursor ion intensity of 500 counts, two or more peptide sequences per protein and a minimum of seven matching fragment ions. Trypsin selectivity was specified allowing for 1 missed cleavage event and variable methionine oxidation. Using a decoy-database method, a score threshold was calculated at the 5% false-discovery rate. Confidence in the protein identification is also increased for those that were identified against both RefSeq and Uniprot Aph databases.

Analyses of differential Aph gene expression over the course of infection. Synchronous infections of HL-60 cells with Aph DC organisms were established. Indirect immunofluorescence microscopic examination of aliquots recovered at 24 h confirmed that ≥60% of HL-60 cells contained morulae and that the mean number of morulae per cell was 2.8+0.6. The infection time course proceeded for 36 h at 37° C. in a humidified atmosphere of 5% CO₂. At the appropriate time-point, aliquots were removed and processed for RNA isolation and RT-qPCR was performed using gene-specific primers. Relative transcript levels for each target were normalized to the transcript levels of the Aph 16S rRNA gene (Aph_1000) using the 2^{-ΔΔC}_{T} method.

Transmission feeding of Aph infected Ixodes. scapularis nymphs. Aph-infected I. scapularis nymphs were obtained from a tick colony maintained at Yale University (New Haven, Conn.). To propagate Aph-infected ticks, clean I. scapularis larvae were fed on Aph-infected C3H/HeJ mice, and the larvae were allowed to molt to nymphs. Infection was confirmed by testing 10% of each tick batch by PCR of the Aph 16S rRNA gene. Ticks were incubated at 23° C. with 85% relative humidity between feedings. To collect transmission-fed nymphs, groups of 20-25 infected tick nymphs were placed to feed on clean 5-6 week-old C3H/HeJ female mice and removed after 24, 48, or 72 hours of feeding. Salivary glands dissected from 2-3 ticks were pooled into a tube of RLT buffer and frozen at −80° C., prior to RNA extraction with the Qiagen RNEasy Kit (Qiagen, Calif.). Unfed ticks were dissected and RNA extracted from combined salivary glands and midguts. RT-qPCR was performed as described above.

Recombinant protein expression and purification and antisera production. Aph genes of interest were amplified using gene-specific primers and Platinum Pfx DNA polymerase (Invitrogen). Amplicons were cloned into pENTR/TEV/D-TOPO (Invitrogen) as described [83] to yield pENTR-candidate gene entry plasmids containing the genes of interest. Plasmid inserts were verified and recombination of the candidate gene insert downstream of and in frame with the gene encoding GST was achieved using the pDest-15 vector (Invitrogen). In some cases plasmids encoding GST-OmpA or GST-Asp14 were subjected to PCR mutagenesis using the Stratagene Quick Change kit according to the manufacturer's instructions for the purpose of inserting DNA segments encoding five-amino acid linkers or substituting the alanine codon for a specific OmpA or Asp14 amino acid. Expression and purification of GST-OmpA, GST-Asp14, and GST-Msp5 and generation of murine polyclonal antisera against each protein were performed as described. KLH-conjugated peptides corresponding to OmpA amino acids 23-40, 41-58, or 59-74 or Asp14 amino acids 101-112 or 113-124 were synthesized by and used for raising rabbit polyclonal antiserum against each peptide by New England Peptides (Gardner, Mass.).

Antibodies, western blot analyses, and spinning disk confocal microscopy. Antisera generated in this study and previous studies targeted OmpA, Asp14, Msp5, APH_0032 [61], APH_1387 [83], Msp2 (P44), and Asp55 and Asp62. The latter two antibodies were gifts from Yasuko Rikihisa of The Ohio State University (Columbus, Ohio). Anti-Msp2 (P44) mAb 20B4 [84,85] was a gift from J. Stephen Dumler of The Johns Hopkins University (Baltimore, Md.). Western blot analyses were performed. Aph infected HL-60 cells were processed and analyzed via indirect immunofluorescence using spinning disk confocal microscopy.

Surface trypsin digestion of intact Aph DC organisms. Intact DC bacteria were incubated at a 10:1 ratio of total protein to trypsin (Thermo Scientific, Waltham, Mass.) in 1× PBS or vehicle alone at 37° C. After 30 min, phenylmethanesulfonyl fluoride (Sigma) was added to a final concentration of 2 mM. Bacteria were pelleted at 5,000 g for 10 min, after which pellets were resuspended in urea lysis buffer and processed. Lysates of trypsin- and vehicle-treated Aph organisms were fractionated by SDS-PAGE, Western-blotted, and screened with antibodies targeting OmpA, Asp14, Asp55 [33], Msp5, Msp2 (P44), and APH_0032.

Flow cytometry. 1×10⁷ HL-60 cells infected with either transgenic HGE1 organisms expressing GFP or wild-type Aph bacteria were mechanically lysed followed by differential centrifugation to pellet host cellular debris. GFP-positive Aph organisms and remaining host cellular debris were pelleted, followed by resuspension in PBS containing equivalent amounts of a 1:25 dilution of preimmune mouse serum, mouse anti-Asp14 or anti-OmpA, or secondary antibody alone. Antibody incubations and wash steps were performed. For FACS analyses, samples were analyzed on a FACSCanto II Flow Cytometer (Becton Dickinson, Franklin Lakes, N.J.). 1×10⁸ events, which corresponded to individual Aph organisms and host cellular debris, were collected in the VCU Flow Cytometry and Imaging Shared Resource Facility. Post data-acquisition analyses were performed using the FCS Express 4 Flow Cytometry software package (De Novo Software, Los Angeles, Calif.).

In silico analyses. The MEMSAT-SVM algorithm (bioinf.cs.ucl.ac.uk/psipred) was used to predict the membrane topology of Aph OmpA. Predicted signal sequences for Anaplasma spp., Ehrlichia spp., and O. tsutsugamushi OmpA proteins were determined using TMPred (www.ch.embnet.org/software/TMPRED_form). Alignments of OmpA sequences (minus the predicted signal sequences) were generated using CLUSTAL W. The tertiary structure for Aph OmpA was predicted using the PHYRE² (Protein Homology/analogy Recognition Engine, version 2.0) server (see the website at sbg.bio.ic.ac.uk/phyre2). To assess how OmpA potentially interacts with sLe^{x}, the OmpA tertiary structure predicted by PHYRE² was docked with the crystal structure for sLe^{x} using the autodock vina algorithm.

Assay for inhibition of Aph binding and infection. For antibody blocking studies, infection assays were performed as described, except that host cell-free Aph organisms were incubated with heat-killed mouse polyclonal antiserum targeting GST, GST-Asp14, or GST-OmpA (10-200 ug/ml) or rabbit polyclonal anti-OmpA (targeting OmpA aa23-40, aa43-58, or aa59-74) and/or anti-Asp14 peptide serum (targeting Asp14 aa98-112 or aa 113-124) for 30 min, after which the bacteria were added to HL-60 cells in the continued presence of antiserum for 1 h. Unbound bacteria were removed and aliquots of host cells were examined for bound Aph organisms using indirect immunofluorescence microscopy. The remainders of the samples were incubated for 48 h, after which host cells were examined for the presence of morulae using indirect immunofluorescence microscopy. For recombinant protein blocking studies, RF/6A or HL-60 cells were incubated with 4 μM GST; GST-Asp14; GST-OmpAor GST APH_1387_{Δ1-111} at 37° C. for 1 h. Host cells were washed with PBS to remove unbound proteins, fixed with paraformaldehyde for 1 h, and permeabilzed with ice-cold methanol for 30 min. Protein binding to host cells was assessed by indirect immunofluorescence microscopy using rabbit anti-GST antibody (Invitrogen). For blocking studies, host cells were incubated with recombinant proteins for 1 h after which Aph organisms were added for an additional 24 h. Unbound bacteria were removed and the samples were incubated for 48 h followed by immunofluorescence microscopy analysis for the presence of morulae.

Statistical analyses. The Student's t test (paired) performed using the Prism 4.0 software package (Graphpad; San Diego, Calif.) was used to assess statistical significance. Statistical significance was set at p<0.05.

### Example 1

Neutravidin affinity purification of biotinylated Aph DC surface proteins and two-dimensional-liquid chromatography tandem mass spectrometry (2D-LC/MS-MS) proteome analysis identifies novel outer membrane protein candidates. DC bacteria were purified to remove the majority of contaminating host cellular debris. DC surface proteins labeled by Sulfo-NHS-SS-Biotin were recovered by neutravidin affinity chromatography (data not shown). Aliquots of input host cell-free DC lysate, affinity-captured DC surface proteins, neutravidin beads plus unlabeled DC whole cell lysate (lane 3), and neutravidin beads alone were resolved by SDS-PAGE followed by silver staining.

Because the Aph DC is the adherent and infectious form and the complement of DC surface proteins is unknown, we set out to identify DC surface proteins. Aph infected HL-60 cells were sonicated to liberate the bacteria from host cells and destroy fragile RC organisms. Electron microscopic examination of sonicated samples confirmed the presence of DC, but not RC bacteria, along with host cellular debris (data not shown). DC organisms were surface-labeled and biotinylated proteins were captured by chromatography. Aliquots of affinity-captured DC proteins, input host cell-free DC lysate, neutravidin beads plus unlabeled DC whole cell lysate, and neutravidin beads alone were resolved by SDS-PAGE followed by silver staining (data not shown). Comparison of the banding patterns of the input lysate and eluate revealed enrichment for many proteins. With the exception of proteins of 44 kDa and 70 kDa, both of which were recovered in low abundances, non-biotinylated DC whole cell lysate proteins did not bind to neutravidin beads.

Eluted proteins were subjected to 2D-LC/MS-MS proteomic analysis. Resulting data were searched against 2 Aph-specific FASTA databases (RefSeq and Uniprot sources) using Protein Lynx Global Surveyor (PLGS) software. Table 5 summarizes a total of 56 identified Aph proteins, 47 of which were identified in both the RefSeq and UnitProt sources.

### Table 5. Aph DC Proteins Recovered Post-Surface Labeling and Affinity Chromatography Analyzed by 2D-NanoLC/Tandem MS Protein Analysis

All proteins for which at least two peptides were identified from either RefSeq or UnitProt and scored above a 5% false-discovery cutoff are listed. Three protein identifications from each search result are likely false-positives, and are most probably among those found on one search result. Nine proteins had previously been delineated as being surface-localized, thereby validating the efficacy of our approach. Ten paralogs of the major surface protein 2 [Msp2 (P44)] family were identified, eight of which yielded the highest PLGS scores.

### Example 2

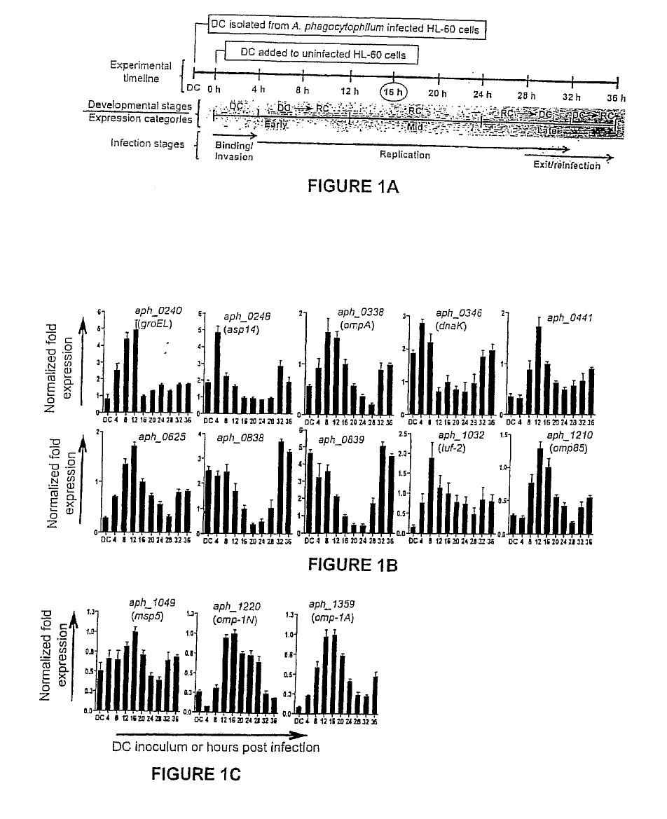

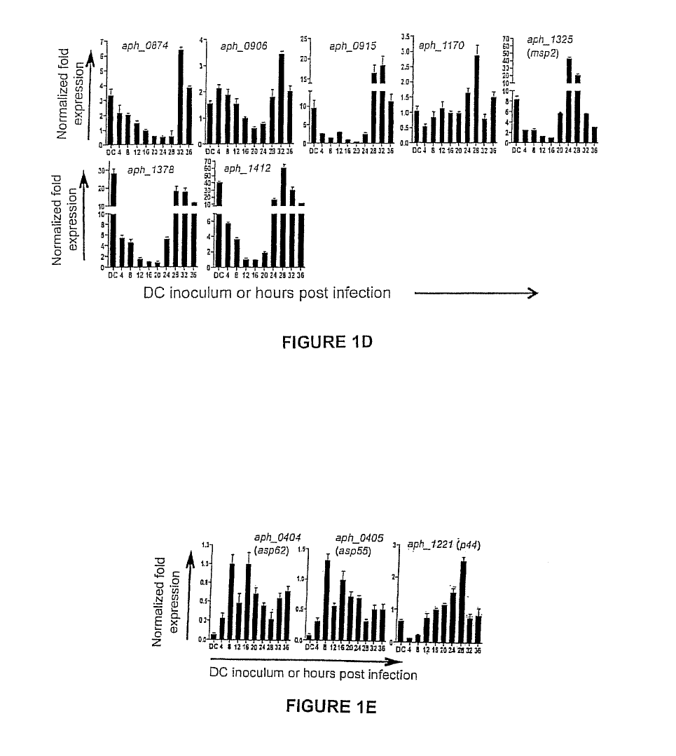

Selection of Aph OMP candidates for further study. FIG.1A illustrates the experimental timeline relative to the the infection cycle and stages of Aph organisms during infection of a host. DC organisms were used to synchronously infect HL-60 cells and the infection proceeded for 36 h, a time period that allows for the bacteria to complete their biphasic developmental cycle and reinitiate infection. Total RNA was isolated from the DC inoculum and from infected host cells at several postinfection time points. RT-qPCR was performed using gene-specific primers. Relative transcript levels for each target were normalized to Aph 16S rRNA gene transcript levels using the 2^{-ΔΔC}_{T} method. To determine the relative transcription of OMP candidate genes between RC and DC organisms, normalized transcript levels of each gene per time point (shown in FIG. 1B-D) were calculated as the fold-change in expression relative to expression at 16 h (encircled in the experimental timeline in FIG. 1A), a time point at which the Aph population consists exclusively of RC organisms. (FIG. 1A) Diagram of the experimental design highlighting the time points at which RNA was isolated, the Aph biphasic developmental and infection stages, and the expression categories into which each gene of interest was classified based on its expression profile. (FIGS. 1B-D) RT-qPCR results for each OMP candidate-encoding gene of interest are grouped as (1B) early stage, (1C) mid stage, and (1D) late stage depending on when during the course of infection they are most highly expressed. (FIG. 1E) RT-qPCR results for control genes. The data in FIGS. 1B-E are the means and standard deviations of results for triplicate samples and are representative of two independent experiments that yielded similar results.

Several proteins were selected for differential gene expression analysis over the course of Aph infection. Asp14, APH_0625, and APH_0874 were chosen because they were hitherto hypothetical proteins. For the remainder of this paper, we will refer to “hypothetical” proteins for which we have demonstrated expression as “uncharacterized” proteins. APH_1049 (Msp5), APH_1210 (Omp85), and APH_1359 (Omp-1A) were selected because, even though they are confirmed Anaplasma spp. proteins, their differential gene expression patterns have yet to be studied. APH_0240 (chaperonin GroEL), APH_0346 (DnaK), and APH_1032 (elongation factor Tu) were chosen because, even though these proteins play housekeeping roles, they have also been identified as surface proteins of Aph and other bacterial species and/or have been linked to bacterial adhesion.

A limitation of the surface biotinylation-affinity proteomics method is that it will not identify surface proteins that are inaccessible to the cross-linker, either due to a lack of free amine groups for cross-linking or due to excessive distance from the bacterial surface to which it extends relative to the length of the cross-linker. Also, detergents may not fully extract integral membrane proteins or protein complexes. Lastly, a surface protein that is in low abundance may not be in sufficient quantity to be detected even if biotinylated. We rationalized that Aph genes upregulated during colonization of mammalian versus tick cells are important for infection of mammalian cells. Therefore, as a complementary approach, we selected 9 candidate genes that are known to be preferentially expressed during infection of HL-60 cells and endothelial cells versus infection of ISE6 (immortalized I. scapularis embryonic) cells and are predicted by the CELLO subcellular prediction server to localize to the Aph outer membrane. These candidates, which were not detected by our or a previous surface proteomics study, are OmpA (homologous to peptidoglycan-associated lipoprotein [Pal]; conserved among most Gram-negative bacteria), APH_1220 (Omp-1N), APH_1325 (Msp2), APH_0838, APH_0839, APH_0906, APH_0915, APH_1378, and APH_1412. We also selected aph_0441 and aph_1170, because they encode previously detected, but uncharacterized Aph surface proteins. The SignalP 3.0 server predicts 9 of the 20 candidates—OmpA, Omp-1a, Omp-1N, Omp85, Msp2, Msp5, APH_0441, APH_0915, and APH_1378—to carry N-terminal signal peptide sequences. The TMPred algorithm (see the website at ch.embnet.org/software/TMPRED_form.html) predicts that all candidates except for Asp14 and APH_1412 carry one or more transmembrane domains.

### Example 3

Differential transcription profiling of omp candidate genes throughout the Aph infection cycle. To gain insight into the transcription of the 20 genes of interest during the Aph infection cycle, we synchronously infected HL-60 cells with DC organisms and allowed the infection to proceed in order for the bacteria to complete their biphasic developmental cycle and initiate a second round of infection. We isolated total RNA from DC organisms used as the inoculum and from bacteria recovered at several post-infection time points. RT-qPCR was performed on total RNA using gene-specific primers. Relative transcript levels for each target were normalized to Aph 16S rRNA gene (aph_1000) transcript levels using the 2^{-ΔΔC}_{T} method. To facilitate identification of genes that are up-regulated in the infectious DC form compared to the non-infectious RC form, normalized transcript levels for each gene per time point were calculated as the fold-change in expression relative to expression at 16 h, a time point at which the Aph population consists exclusively of RC organisms.

Genes of interest were classified as early (0-12 h), mid (12-24 h), or late stage (24-36 h) (FIG. 1A). The early stage correlates with DC adhesion and invasion, DC to RC differentiation, and initiation of RC replication. Early stage gene transcription increased at 4 h and peaked at 8 h or 12 h, except for asp14 and aph_0346, both of which peaked at 4 h (FIG. 1B). Expression levels of all early stage genes began to increase again between 28 and 36 h, which correspond to the period during which Aph RC organisms differentiate to DC organisms and initiate the second round of infection. Mid stage gene expression, which coincides with a period of extensive Aph replication, peaked at 16 h (FIG. 1C). Late stage genes were upregulated between 24 and 36 h (FIG. 1D), a period that correlates with the conversion of RC to DC organisms, DC exit, and initiation of the second round of infection. All target mRNAs were detected in host cell-free DC organisms (FIG. 1). Transcript levels of asp14, aph_0346, aph_0838, aph_0839, aph_0874, aph_0915, aph_1378, aph_1412, and msp2 were more abundant in DC bacteria used as the inoculum than in RC bacteria at 16 h. Because msp2 (P44), asp62, and asp55 encode confirmed Aph surface proteins and because the latter two constitute an operon, these genes were analyzed as controls. Coincident with the kinetics of the infection cycle, msp2 (p44) transcription steadily increased from 4 to 28 h, after which it pronouncedly declined by 32 h. The transcriptional profiles of asp55 and asp62 were highly similar, which reinforces the accuracy of the expression data obtained for all genes.

### Example 4

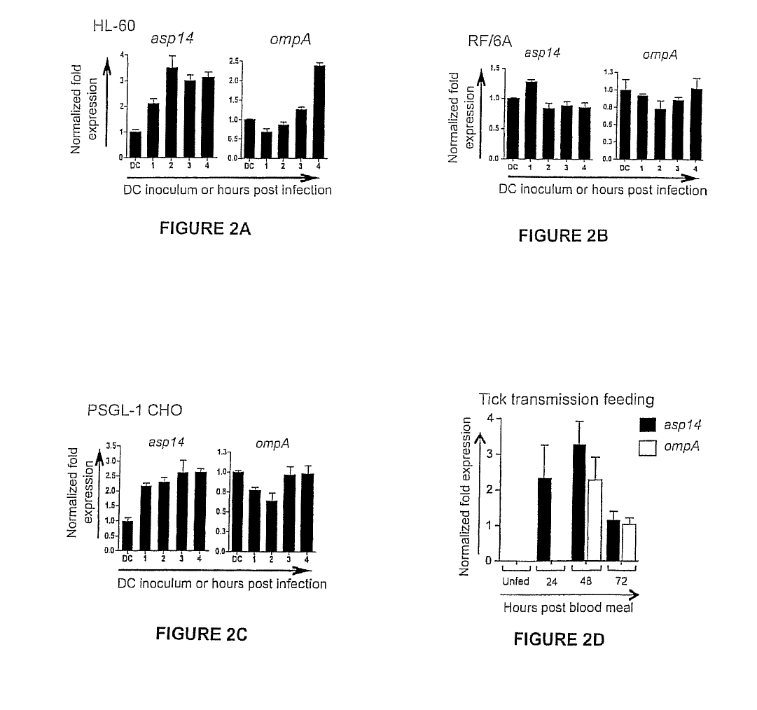

Aph transcriptionally upregulates ompA and asp14 during binding and invasion of myeloid but not endothelial cells. It takes up to four hours for the majority of bound Aph organisms to enter and reside within nascent host cell-derived vacuoles. Thus, genes that are upregulated between 0 and 4 h and in the initial hours following bacterial entry conceivably encode products that are important for invasion and/ or establishing infection. Of all genes examined, asp14 is the most abundantly expressed at 4 h (FIG. 1B-E), and asp14 and ompA exhibit the most abundant non-DC to RC-normalized transcript levels (data not shown). Accordingly, we more closely examined the expression profiles of ompA and asp14. Differential expression analyses of ompA and asp14 during Aph invasion of HL-60 and RF/6A cells, during Aph binding to PSGL-1 CHO cells, and during transmission feeding of Aph infected I. scapularis ticks is shown in FIG. 2A-C. Aph organisms were incubated with HL-60 (2A), RF/6A (2B), and PSGL-1 CHO cells (2C) for 4 h, a period that is required for bacterial adherence and for ≥90% of bound bacteria to invade host cells. Aph cannot invade PSGL-1 CHO cells. Total RNA was isolated from the DC inoculum and from host cells at 1, 2, 3, and 4 h post-bacterial addition. (2D) Aph infected I. scapularis nymphs were allowed to feed on mice for 72 h. Total RNA was isolated from the salivary glands of uninfected and transmission fed ticks that had been removed at 24, 48, and 72 h post-attachment. Total RNA was isolated from combined salivary glands and midguts from unfed ticks. (2A-2D) RT-qPCR was performed using gene-specific primers. Relative transcript levels for asp14 and ompA were normalized to Aph 16S rRNA gene transcript levels. The normalized values in FIGS. 2A-C are presented relative to asp14 or ompA transcript levels of the DC inoculum. Data are the means and standard deviations of results for triplicate samples and are representative of two independent experiments that yielded similar results.

Aph DC bacteria were added to HL-60 and RF/6A cells, after which RT-qPCR was performed on total RNA isolated at 1, 2, 3, and 4 h. RNA isolated from the DC bacterial inoculum served as a reference control. asp14 was upregulated at all time points during adhesion and invasion of HL-60 cells and exhibited a maximal increase at 2 h, whereas ompA demonstrated a maximal increase at 4 h (FIG. 2A). Neither ompA nor asp14 was upregulated during binding and invasion of endothelial cells (FIG. 2B).

### Example 5

Aph engagement of PSGL-1 promotes upregulation of asp14, but not ompA. We next examined whether Aph binding to PSGL-1 upregulates either asp14 or ompA. Chinese hamster ovary cells transfected to express PSGL-1 (PSGL-1 CHO cells) are ideal models for studying Aph-PSGL-1 interactions because they support Aph binding, while untransfected CHO cells that lack PSGL-1 expression do not. Thus, Aph binding to PSGL-1 CHO cells occurs exclusively through bacterial engagement of PSGL-1. DC bacterial binding to PSGL-1 CHO cells upregulated asp14, but not ompA (FIG. 2C).

### Example 6

Aph upregulates ompA and asp14 during I. scapularis transmission feeding. Aph genes that are induced during the bloodmeal of infected I. scapularis ticks are presumably important for establishing infection in mammals. We examined ompA and asp14 expression in Aph infected I. scapularis nymphs during transmission feeding on naive mice. Transcripts for neither ompA nor asp14 were detected in unfed Aph infected nymphs (FIG. 2D). Both asp14 and ompA were induced during transmission feeding, being first detected at 24 h and 48 h, respectively.

### Example 7

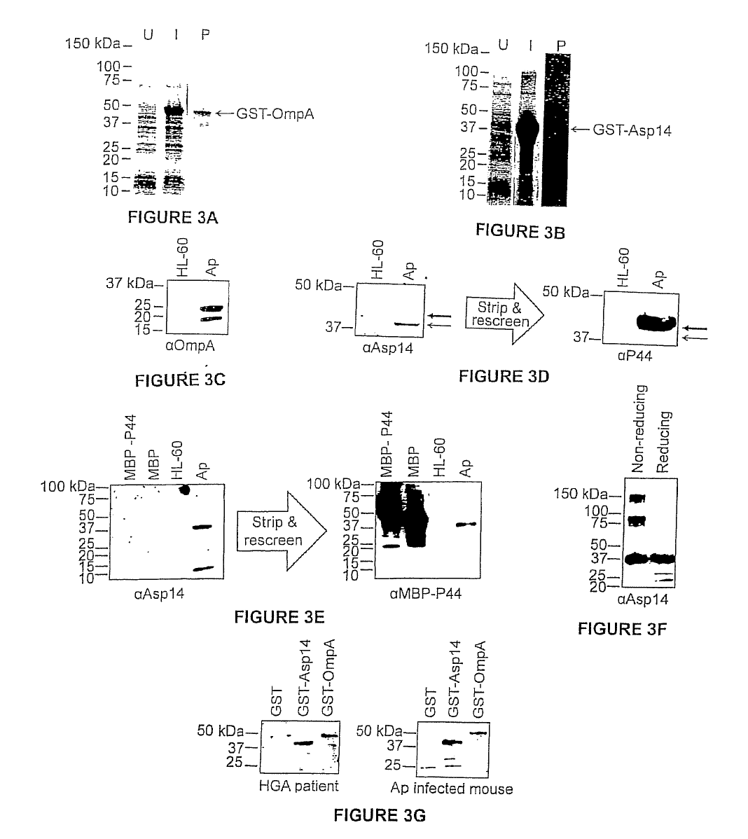

Aph expresses OmpA and Asp14 during infection of HL-60 cells and during murine and human infection. As illustrated in FIGS. 3A and B, whole cell lysates of E. coli (U), E. coli induced (I) to express GST-OmpA (FIG. 3A) or GST-Asp14 (FIG. 3B), and GST-OmpA (3A) or GST-Asp14 (3B) purified (P) by glutathione sepharose affinity chromatography were separated by SDS-PAGE and stained with Coomassie blue. (FIGS. 3C and D) Western blot analyses in which mouse anti-OmpA (αOmpA; raised against GST-OmpA) and αAsp14 (raised against GST-Asp14) were used to screen whole cell lysates of uninfected HL-60 cells and Ap organisms. The blot in FIG. 3D was stripped and rescreened with anti-Msp2 (P44) (aP44). The thin and thick arrows denote Asp14 and Msp2 (P44), respectively. (FIG. 3E) Western blotted MBP-P44, MBP, and whole cell lysates of uninfected HL-60 cells and Aph organisms were screened with αAsp14. The blot was stripped and rescreened with anti-MBP-P44. (FIG. 3F) GST-Asp14 was resolved by SDS-PAGE under non-reducing and reducing conditions, Western-blotted, and screened with αAsp14. (FIG. 3G) Western-blotted GST-OmpA, GST-Asp14, and GST were screened with sera from an HGA patient and an experimentally infected mouse.

The coding regions of ompA (excluding the signal sequence; 19.9 kDa) and asp14 (13.8 kDa) were cloned and expressed in E. coli as N-terminal glutathione-S-transferase (GST)-tagged fusion proteins designated as GST-OmpA and GST-Asp14, respectively (FIGS. 3A and B). After glutathione-Sepharose affinity chromatography, purified GST-OmpA and GST-Asp14 appeared as 46.0- and 39.8-kDa bands, respectively, upon SDS-PAGE. Each fusion protein was used to immunize mice. Polyclonal anti-OmpA antisera recognized proteins of 22.1 kDa and 19.9 kDa, which correspond to OmpA preprotein and mature OmpA, respectively, in an Aph lysate but not an uninfected HL-60 cell lysate (FIG. 3C). In addition to the anticipated 13.8 kDa band, anti-Asp14 detected a band of approximately 42 kDa in a lysate of Aph, but not uninfected HL-60 cells (FIG. 3E). Anti-Asp14 occasionally detected another band of approximately 28 kDa on blots of Aph lysates (data not shown). Even though the 42-kDa band is close in size to that anticipated for Msp2 (P44), anti-Asp14 failed to recognize Aph-derived maltose binding protein (MBP)-tagged Msp2 (P44) (FIGS. 3D and E). An amino acid sequence alignment of Asp14 with Msp2 (P44)-23, the most abundantly expressed Msp2 (P44) paralog of the Aph NCH-1 strain [56,57], revealed no considerable stretches of homology (data not shown). GST-Asp14 multimerizes when fractionated by non-denaturing SDS-PAGE (FIG. 3F). Thus, the 28- and 42-kDa bands in the Aph lysate recognized by anti-Asp14 are presumably multimeric complexes that consist exclusively of or contain Asp14. HGA patient serum and Aph infected mouse serum recognize GST-OmpA and GST-Asp14 (FIG. 3G), signifying that Aph expresses OmpA and Asp14 during human and murine infection.

### Example 8

OmpA is differentially expressed by Aph during infection of mammalian versus tick cells, while Asp14 is expressed during infection of both mammalian and tick cells. Because Aph infects myeloid cells, endothelial cells, and I. scapularis cells in vivo and in vitro, we examined Asp14 and OmpA expression during infection of HL-60 cells, RF/6A cells, and ISE6 cells, (data not shown). Aph infected HL-60, RF/6A, and ISE6 cells were fixed and viewed by confocal microscopy to determine immunoreactivity with antibodies against Msp2 (P44) (major surface protein; used to identify bacteria), OmpA, or Asp62 (confirmed surface protein). Both OmpA and Asp62 staining yield comparable ring-like bacterial surface staining patterns. Results described are the means and standard deviations of results of at least two separate experiments. At least 200 Msp2 (P44)-positive morulae were scored for Asp14 and OmpA per condition. Confocal microscopic examination using anti-Asp14 or anti-OmpA in conjunction with antiserum against constitutively expressed Msp2 (P44) revealed that 100.0% of morulae (intravacuolar Aph colonies) in each of the three cell lines was Asp14-positive. OmpA was detected in 100.0% and 48.6+15.9% of moruale in HL-60 and RF/6A cells, respectively, but was detected in only 7.0±3.5% of morulae in ISE6 cells (results were statistically significant, p<0.001). Anti-OmpA binding to intracellular Aph organisms yielded a ring-like staining pattern on the periphery of each bacterium that overlapped with signal corresponding to the confirmed surface protein, Msp2 (P44)(data not shown). The anti-OmpA staining pattern was similar to that of another confirmed Aph surface protein, Asp62. Anti-Asp14 staining was more uniformly distributed over the bacterial cells and exhibited partial overlap with Msp2 (P44) (data not shown).

### Example 9

Surface localization of OmpA and Asp14.

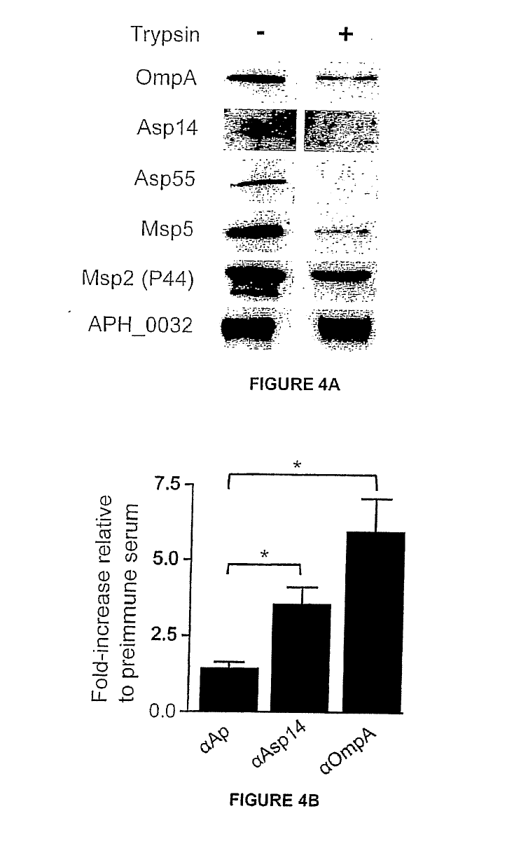

To assess surface presentation of OmpA and Asp14, intact Aph DC organisms were incubated with trypsin followed by solubilization, western blotting, and screening with anti-OmpA or anti-Asp14 to determine if immunoaccessible domains of either target protein are presented on the bacterial surface, shown in FIGS. 4A and B. In FIG. 4A, Intact DC bacteria were incubated with trypsin or vehicle control, lysed in RIPA buffer, fractionated by SDS-PAGE, and immunoblotted. Western blots were screened with antisera targeting OmpA, Asp55, Msp5, Asp14, Msp2 (P44), or APH_0032. Data are representative of two experiments with similar results. In FIG. 4B, Transgenic Aph organisms expressing GFP were incubated with preimmune mouse serum, mouse anti-Asp14 or anti-OmpA, or serum recovered from an Aph infected mouse. Primary antibodies were detected with anti-mouse IgG conjugated to Alexa fluor 647. Flow cytometry was used to determine the percentage of Alexa fluor 647- and GFP-positive DC organisms per sample. The fold-increases in the percentages of Alexa fluor 647-positive, GFP-positive DC organisms for each sample relative to preimmune serum are provided. Results presented are the means±SD of three experiments. Statistically significant (*, p<0.05) values are indicated. Positive control antisera targeted Asp55, Msp2 (P44), and Msp5. Negative control antiserum was specific for APH_0032, which is an Aph effector and is not a surface protein. Anti-Asp55 is specific for a peptide epitope of a surface-exposed loop of the target protein. Considerably less detection of Asp55, OmpA, Asp14, and Msp5 was observed for trypsin-treated than for vehicle control-treated bacteria, whereas Msp2 (P44) signal intensity was partially reduced and no loss in APH_0032 signal resulted (FIG. 4A). As a complementary approach to verify surface presentation of OmpA and Asp14, transgenic Aph DC organisms expressing GFP were recovered from sonicated HL-60 cells and screened with anti-OmpA, anti-Asp14, or control antisera using flow cytometry. Serum from an Aph infected mouse recognized 1.9±0.8-fold more organisms than preimmune mouse serum (FIG. 4B). Anti-OmpA and anti-Asp14 recognized 5.0±2.9- and 4.9±2.7-fold more Aph organisms expressing GFP than preimmune mouse serum (FIG. 4B).

### Example 10

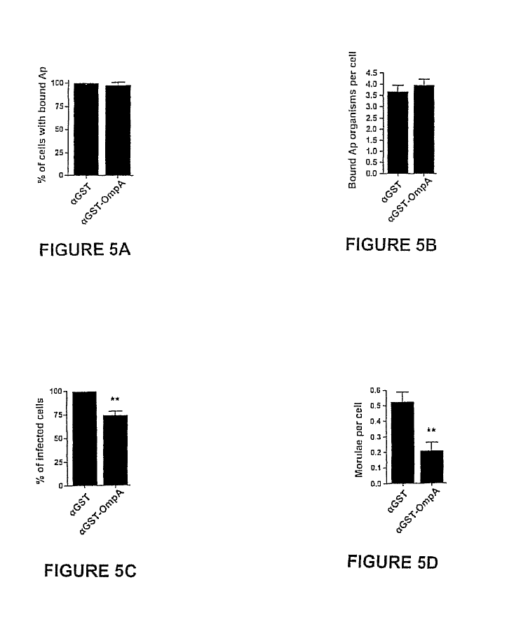

Pretreatment of Aph with anti-OmpA reduces infection of HL-60 cells. Because OmpA is exposed on the Aph surface, we determined if treating DC organisms with heat-inactivated anti-OmpA serum prior to incubation with HL-60 cells alters bacterial adhesion to or infection of host cells. Anti-OmpA had no effect on bacterial adhesion, but significantly reduced infection (FIG. 5A-D). Pretreatment of bacteria with mouse polyclonal anti-GST serum had no effect on binding or infection.

### Example 11

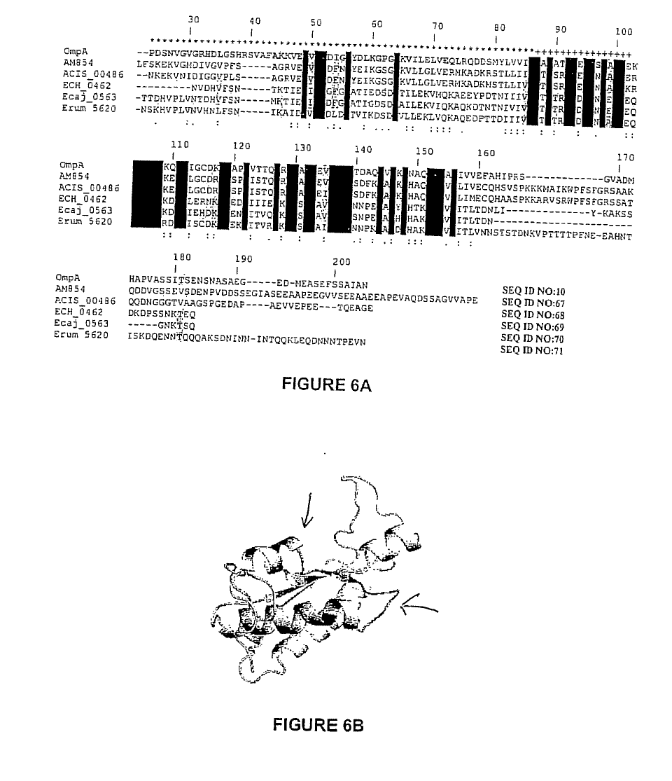

In silico analyses of Aph OmpA and comparisons with homologs from other Anaplasmataceae pathogens. Since anti-OmpA inhibits Aph infection, we hypothesized that OmpA may contribute to infection of host cells. We performed in silico analyses to identify the predicted extracellular region of OmpA, which would putatively contain any receptor-binding domain, and to assess whether this and other regions of OmpA are conserved among its homologs from other Rickettsiales bacteria. The OmpA N-terminal region extending through to amino acid 86 is predicted to comprise the only extracellular domain, and amino acids 87-102 are predicted to form a transmembrane helix (FIG. 6A). A multiple sequence alignment revealed that the Aph OmpA sequence has several shaded stretches that exhibit identity or similarity with its homologs from other Anaplasma spp. and Ehrlichia spp. (FIG. 6A).

The PHYRE² server (see the website at sbg.bio.ic.ac.uk/phyre2) predicts tertiary structures for protein sequences and threads the predicted structures on known crystal structures. The highest scoring model for Aph OmpA that exhibits the greatest amino acid sequence identity with the crystal structure on which it was threaded, Bacillus chorismate OmpA, is presented in FIG. 6B. Amino acids 44-56 are predicted to form a surface-exposed helix and loop, as indicated by arrows. The peptide K[IV]YFDaxK (where “a” and “x” represent a non-polar and any amino acid, respectively), that corresponds to Aph OmpA residues 49-56 is conserved among Anaplasma spp. and Ehrlichia spp. OmpA proteins.

### Example 12

Interactions of GST-OmpA with endothelial cells. We tested if we could detect GST-OmpA binding to RF/6A cells. Since OmpA proteins of Aph and O. tsutsugamushi exhibit regions of identity, O. tsutsugamushi infects endothelial cells, and it is unknown whether O. tsutsugamushi OmpA interacts with endothelial cells, we also assessed whether GST-tagged O. tsutsugamushi OmpA (GST-OtOmpA) bound to RF/6A cells. Negative controls for cellular adhesion were GST alone and GST-tagged APH_1387 amino acids 112-579 (GST-APH_1387₁₁₂₋₅₇₉). APH_1387 is an Aph effector that associates with the bacterium's vacuolar membrane. APH_1387 amino acids 112-579 lack the transmembrane domain that is required for interacting with eukaryotic cell membranes (unpublished observation). GST-OmpA but not GST bound to RF/6A cells (data not shown). Neither GST-APH_1387₁₁₂₋₅₇₉ nor GST-OtOmpA bound the host cells. GST-tagged Aph OmpA binding to RF/6A cells is therefore specific because recombinant form of neither an irrelevant Aph protein nor OmpA derived from another Rickettsiales bacterium binds to RF/6A cells. GST-OmpA binding to RF/6A cells does not involve PSGL-1 or sLe^{x} since antibodies targeting either receptor fail to bind RF/6A cells (data not shown) and a previous report demonstrated that endothelial cells do not express PSGL-1. We examined if preincubating RF/6A cells with GST-OmpA competitively inhibits Aph binding or infection. GST-OmpA but not GST significantly inhibited infection (data not shown). Neither recombinant protein inhibited Aph adhesion (data not shown).

### Example 13

Sialidase and trypsin treatments markedly reduce GST-OmpA binding to host cells. Enzymatic removal of sialic acid residues from myeloid cell surfaces pronouncedly inhibits Aph binding and infection. Sialic acid residues are also important for Aph infection of RF/6A cells, as pretreatment of RF/6A cells with sialidases reduced Aph infection by 52.8±1.4% (data not shown). The MAL-II lectin recognizes sialic acids that are attached to galactose units via α2,3-linkages. The SNA lectin preferentially binds to sialic acid attached to galactose in an α2,6-linkage. Sialidase treatment abolished MAL-II binding and markedly reduced SNA binding, indicating that the sialidase cocktail completely removed α2,3-linked sialic acids and partially removed α2,6-linked sialic acids. GST-OmpA did not bind as well to RF/6A cells that had been incubated in the vehicle control buffer as compared to other buffers. Nonetheless, GST-OmpA binding to sialidase-treated cells was reduced. These results suggest that OmpA recognizes α2,3-linked sialic acids but is also capable of interacting with α2,6-linked sialic acids. Pretreatment of RF/6A cells with trypsin, which would effectively digest protein and glycoprotein receptors, including terminally sialylated glycoproteins, nearly eliminated GST-OmpA binding.

### Example 14

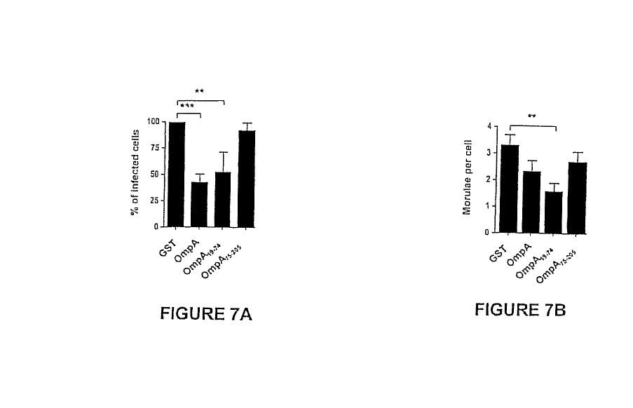

GST-OmpA competitively inhibits Aph infection of HL-60 cells. To define the relevance of OmpA to Ap hinfection of human myeloid cells and to delineate the OmpA region that is critical for cellular invasion, we examined if preincubating HL-60 cells with GST-OmpA or fragments thereof inhibits infection by Aph DC organisms. GST-tagged full-length OmpA and OmpA₁₉₋₇₄, which comprises the majority of the predicted extracellular domain, but not GST-OmpA₇₅₋₂₀₅ or GST alone had no effect on adhesion (data not shown), but significantly inhibited infection (FIGS. 7A and B).

### Example 15

GST-OmpA inhibits Aph binding to sLe^{x}-capped PSGL-1. Aph binding to the α2,3-linked sialic acid determinant of sLe^{x} is necessary for the bacterium to optimally engage sLe^{x}-capped PSGL-1 and leads to infection of myeloid cells. Since GST-OmpA recognizes α2,3-sialic acid and competitively inhibits Aph infection of HL-60 cells, we rationalized that GST-OmpA binds to α2,3-sialic acid of sLe^{x}. To test this, we incubated PSGL-1 CHO cells with GST-OmpA in an attempt to block Aph access to the α2,3-sialic acid determinant of sLe^{x}-capped PSGL-1 and thereby inhibit bacterial adherence to these cells. As a positive control for preventing bacterial access to the α2,3-linked sialic acid determinant of sLe^{x}, PSGL-1 CHO cells were incubated with CSLEX1. PSGL-1 CHO cells treated with GST or mouse IgM served as negative blocking controls. GST-OmpA reduced Aph binding to sLe^{x}-modified PSGL-1 by approximately 60% relative to GST alone, and this degree of inhibition was comparable to the blocking afforded by CSLEX1 (data not shown).

### Example 16

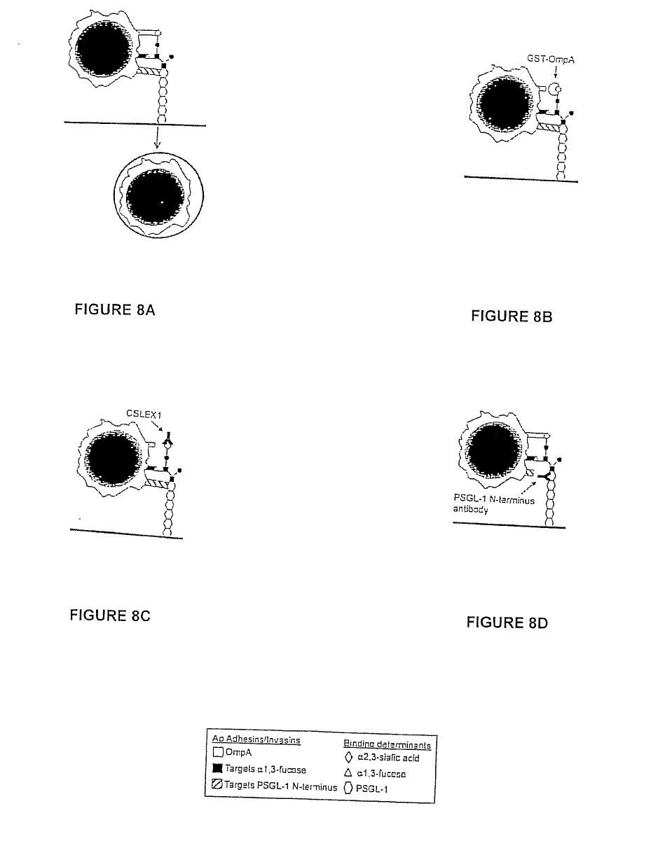

Model for how Aph OmpA interacts with its receptor to promote infection of host cells (FIG. 8A-D). Sialic acid has long been known to be a determinant that is important for Aph infection. This study demonstrates that OmpA targets sialylated glycoproteins to promote Aph infection. Our results fit the model that Aph employs multiple surface proteins to bind three determinants of sLe^{x}-capped PSGL-1 to infect myeloid cells (FIG. 8A). When these data are examined in the context of results obtained from our own studies and others, the respective contributions of sialic acid, α1,3-fucose, and PSGL-1 N-terminal peptide to Aph binding and entry become clearer. Treating myeloid cells with CSLEX1 to block A. phagocytophilum binding to the sialic acid determinant of sLe^{x} markedly reduces infection (FIG. 8C), a phenomenon that is analogous to the inhibitory action of GST-OmpA. Moreover, the inhibitory effects of CSLEX1 and GST-OmpA on Aph binding to PSGL-1 CHO cells are nearly identical. Therefore, while OmpA is capable of binding sialic acid determinants of varied sialylated glycans, its specific interaction with the sialic acid residue of sLe^{x} is important for bacterial entry. GST-OmpA and GST-OmpA₁₉₋₇₄ binding to host cells reduces Aph infection of HL-60 cells by approximately 52 and 57%, respectively, but has no inhibitory effect on bacterial adhesion. Thus, bacterial recognition of the PSGL-1 N-terminus, α1,3-fucose of sLe^{x}, and perhaps sLe^{x}-/PSGL-1-independent interactions that still occur when the OmpA-sialic acid interaction is disrupted facilitate bacterial binding but lead to sub-optimal infection (FIG. 8B). Antibodies that block access to the PSGL-1 N-terminal peptide determinant prevent bacterial binding and infection. Therefore, the collective avidity mediated by OmpA interaction with sialic acid together with Aph recognition of α1,3-fucose is insufficient to promote bacterial adhesion and, consequently, entry in the absence of PSGL-1 recognition (FIG. 8D).

### Example 17

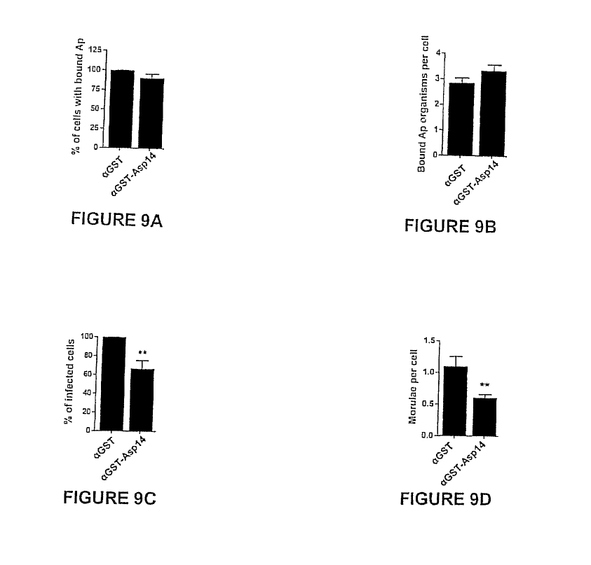

Pretreating Aph with anti-Asp14 inhibits infection of HL-60 cells. Since Asp14 is a surface protein, we examined if incubating Aph DC organisms with heat-inactivated Asp14 antiserum prior to adding them to HL-60 cells inhibited bacterial binding or infection. Anti-Asp14 had no effect on Aph adhesion, but reduced infection by approximately 33% and lowered the mean number of morulae per cell by approximately 54%, (FIGS. 9A-D). Inhibition was specific to Asp14 antiserum, as GST antiserum did not alter bacterial binding or infection.

### Example 18

The Asp14 C-terminal region binds mammalian host cells. Since Asp14 is an exposed outer membrane protein and anti-Asp14 reduces Aph infection, we rationalized that Asp14 may interact with mammalian host cell surfaces to promote infection. To test this possibility and to identify the Asp14 region that is sufficient for optimal adherence, we examined if GST-tagged Asp14 or portions thereof bind to RF/6A cells. GST alone and GST-tagged APH_1387 amino acids 112-579 (GST-APH_1387₁₁₂₋₅₇₉) were negative controls. APH_1387 is an Aph protein that localizes to the pathogen's vacuolar membrane and does not associate with the host cell surface. GST-Asp14 but neither GST nor GST-APH_1387₁₁₂₋₅₇₉ bound to RF/6A cells (FIG. 9A-D). The binding domain is carried on the Asp14 C-terminal half, as GST-Asp14₆₅₋₁₂₄ but not GST-Asp14₁₋₆₄ exhibited binding. GST-Asp14₁₋₁₀₀ and GST-Asp14₁₋₁₁₂ were unable to bind RF/6A cells (data not shown). Thus, Asp14 residues 101-124 contain the minimal region that is sufficient to facilitate adhesion to mammalian cell surfaces.

### Example 19

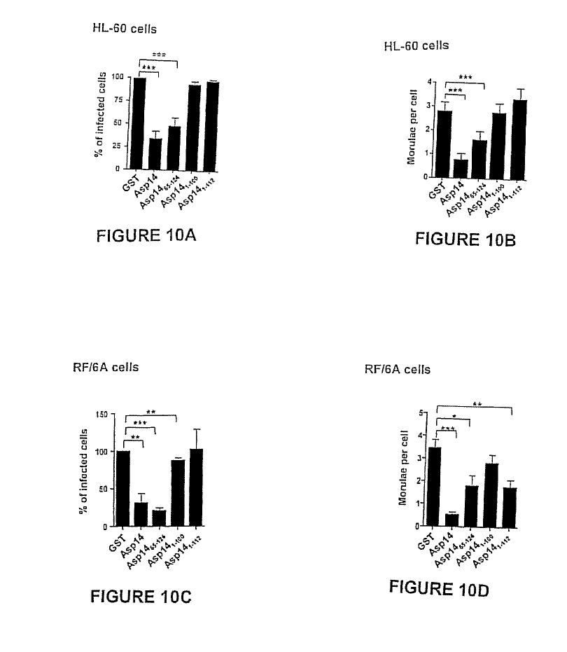

GST-Asp14 requires Asp14 residues 101-124 to competitively inhibit A. phagocytophilum infection of mammalian host cells. We next determined if GST-tagged Asp14 or fragments thereof could inhibit A. phagocytophilum infection. GST-Asp14 and GST-Asp14₆₅₋₁₂₄ each significantly reduced infection of HL-60 and RF/6A cells relative to GST alone (FIG. 10A-D). GST-Asp14₁₋₁₀₀ and GST-Asp14₁₋₁₁₂ had no effect on infection of HL-60 cells (FIGS. 10A and B). GST-Asp14₁₋₁₁₂ did not lower the percentage of infected RF/6A cells, but reduced the mean number of morulae per RF/6A cell comparably to GST-Asp14₆₅₋₁₂₄ (FIGS. 10C and D). Pretreating host cells with GST-Asp14 fusion proteins prior to incubation with bacteria failed to inhibit A. phagocytophilum binding (data not shown). Thus, A. phagocytophilum binding to mammalian host cells is Asp14-independent, but Asp14 is important for bacterial invasion.

### Example 20

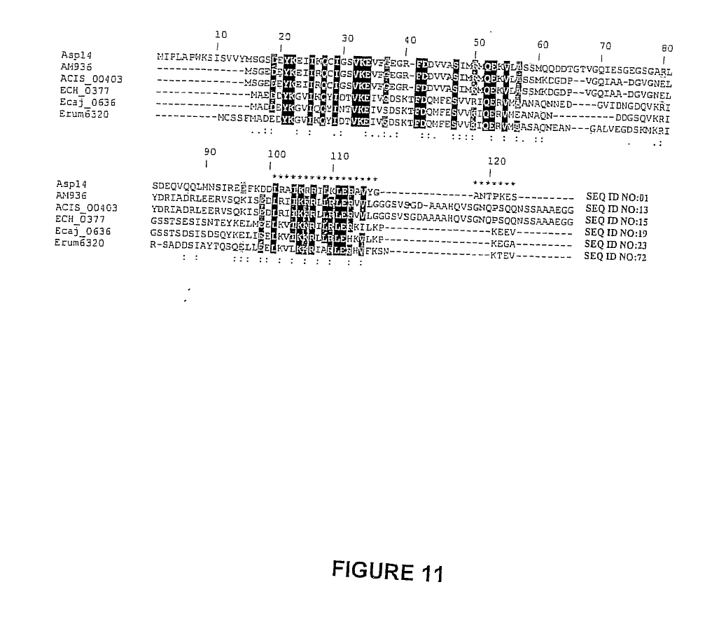

The Asp14 C-terminus is positively charged and residues 101-115 constitute a conserved domain among homologs from Anaplasma and Ehrlichia species. Based on our results, a domain that lies within Asp14 amino acids 101-124 is involved in mediating interactions with host cells that promote A. phagocytophilum infection. To determine if this or any other Asp14 region is conserved among Anaplasmataceae members, we aligned the primary amino acid sequences of Asp14 with its homologs from two A. marginale strains and three monocytotropic Ehrlichia species. Doing so identified two conserved regions, the first of which corresponds to Asp14 amino acids 19-61 (FIG. 11). The second conserved region aligns with Asp14 residues 101-115. The consensus sequence for this region among the Anaplasma and Ehrlichia spp. Asp14 homologs is L[RK]aIKKR[IL]LRLERxV, where “a” and “x” represent a non-polar and any amino acid, respectively. Beginning at tyrosine 116, the Asp14 C-terminus bears no sequence homology to its A. marginale and ehrlichiae counterparts. The Asp14 C-terminus (amino acids 101-124) has a charge of +4.91 despite the entire protein sequence having a charge of −3.10. A similar trend is observed when the charges of the Asp14 homologs' C-termini and entire protein sequences are examined.

### Example 21

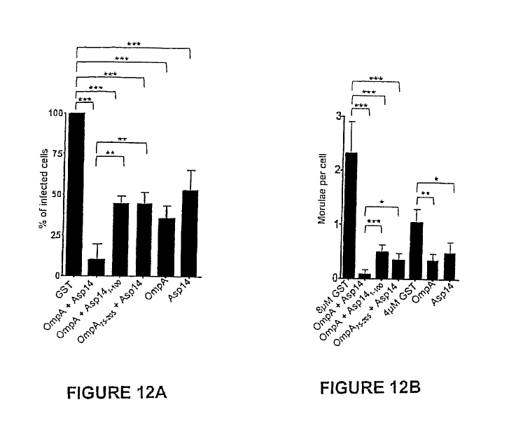

GST-Asp14 and GST-OmpA together more pronouncedly inhibit A. phagocytophilum infection of HL-60 cells than either protein alone. We examined whether we could improve upon the protection against A. phagocytophilum infection afforded by GST-Asp14 or GST-OmpA by pretreating HL-60 cells with both recombinant proteins. Consistent with previous results, 35.5 +7.4% of GST-OmpA-treated and 53.2±11.8% of GST-Asp14-treated HL-60 cells became infected (FIG. 11A). However, HL-60 cells that had been preincubated with both GST-Asp14 and GST-OmpA were better protected against A. phagocytophilum infection, as only 9.9±9.4% of cells developed morulae. To prove that the synergistic reduction in infection was specific to the combinatorial effect of GST-Asp14 and GST-OmpA and not simply due to the presence of excess recombinant protein, we treated HL-60 cells with GST-Asp14 and GST-OmpA, GST-Asp14₁₋₁₀₀ (does not block infection; data not shown) and GST-OmpA, or GST-Asp14 and GST-OmpA₇₅₋₂₀₅ (does not block infection). HL-60 cells treated with GST-Asp14₁₋₁₀₀ and GST-OmpA or GST-Asp14 and GST-OmpA₇₅₋₂₀₅ exhibited reductions in infection and bacterial load comparable to cells treated with GST-Asp14 or GST-OmpA alone (FIGS. 12A and B). HL-60 cells treated with GST-Asp14 and GST-OmpA exhibited an approximate 4.5-fold reduction in the percentage of infected cells relative to cells treated with either GST-Asp14₁₋₁₀₀ and GST-OmpA or GST-Asp14 and GST-OmpA₇₅₋₂₀₅ (FIG. 12A).

### Example 22

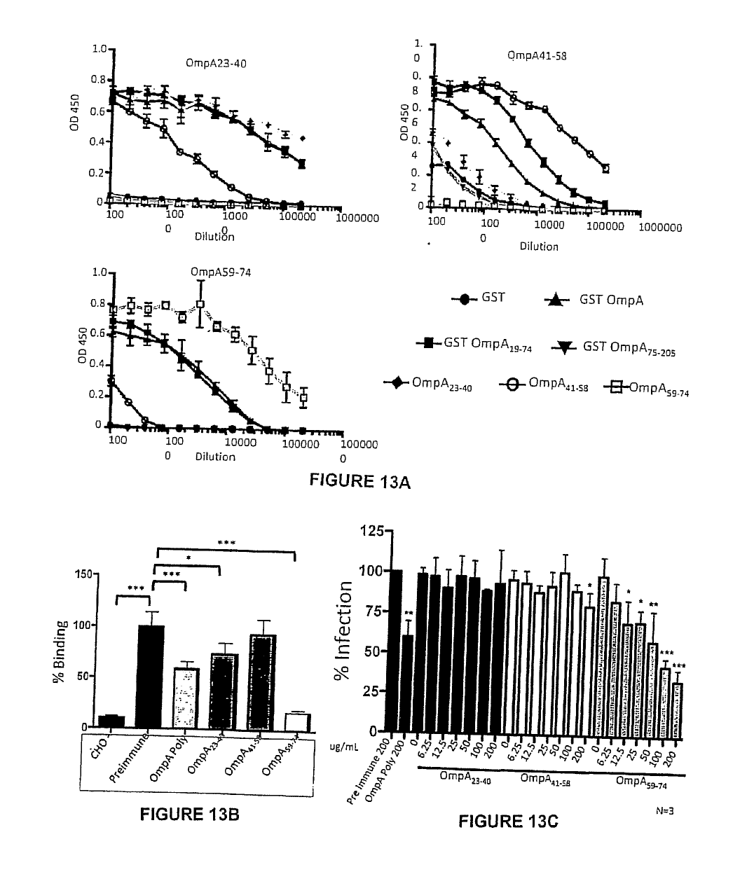

Peptide antisera blocking reveals that the OmpA invasin domain lies within amino acids 59-74. We had rabbit antiserum raised against peptides corresponding to OmpA amino acids 23-40, 41-58, and 59-74. We confirmed by ELISA that each serum is specific for recombinant OmpA and only the peptide against which it was raised (FIG. 13A). Pretreating A. phagocytophilum with serum specific for OmpA₅₉₋₇₄ but neither of the other two peptide sera significantly inhibited A. phagocytophilum infection of host cells in vitro (FIG. 13B). Also, treatment of bacteria with OmpA₅₉₋₇₄ serum but not OmpA₂₃₋₄₀ serum or OmpA₄₁₋₅₈ serum prevents A. phagocytophilum binding to its known receptor, sialylated PSGL-1 (FIG. 13C).

Please note that even though amino acids 59-74 are most important for OmpA to promote infection that amino acids 23-58 are predicted to be presented on the A. phagocytophilum surface and could therefore be a component of a protective vaccine.

### Example 23

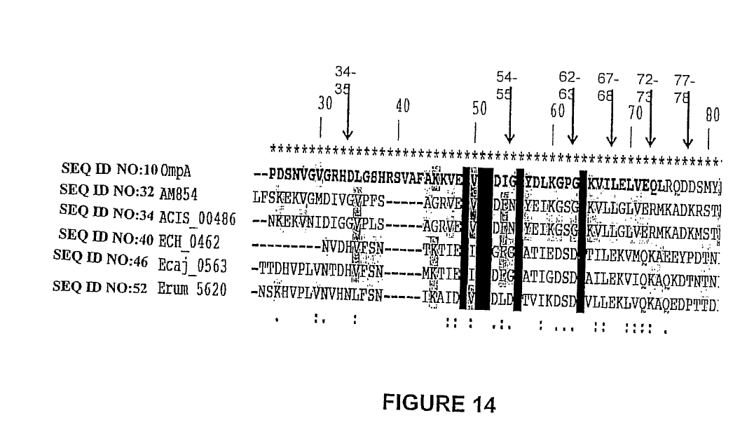

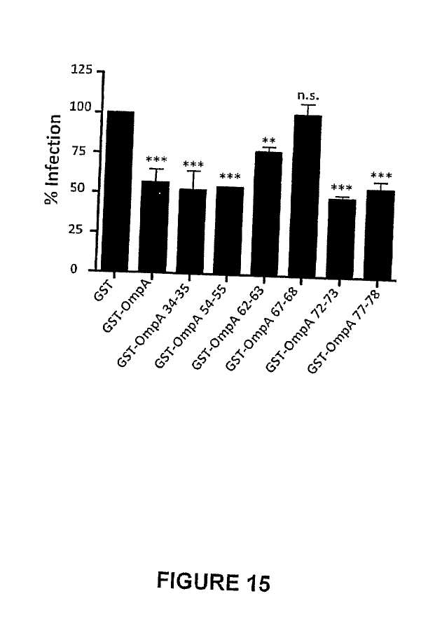

Linker insertions disrupt the ability of GST-OmpA to antagonize A. phagocytophilum infection of mammalian host cells and support that the invasin domain lies within amino acids 59-74. We also generated a series of glutathione-S-transferase (GST)-tagged OmpA proteins having an insertion of 5 amino acids (CLNHL) at defined locations. The purpose of the insertion of the amino acid “linker” was to disrupt any OmpA domain that facilitates binding of the protein to host cell surfaces. Individual plasmids encoding GST-OmpA proteins carrying linker insertions between aspartate 34 and leucine 35; isoleucine 54 and glycine 55; proline 62 and glycine 63; isoleucine 67 and leucine 68; glutamate 72 and glutamine 73; or aspartate 77 and aspartate 78 were generated by PCR mutagenesis of the plasmid encoding GST-OmpA (FIG. 14). E. coli was transformed with each plasmid, induced to express the GST-OmpA proteins, and the proteins were purified by glutathione affinity chromatography. Adding recombinant wild-type OmpA and several OmpA insertion mutant proteins to host cells successfully inhibited A. phagocytophilum infection of host cells (FIG. 15). These data indicate that the OmpA proteins were still able to bind to the OmpA receptor and competitively inhibit bacterial access to the receptor. However, only the GST-OmpA protein bearing a linker insertion between isoleucine 67 and leucine 68 lost the ability to competitively inhibit infection, which indicates that disruption of the region encompassed by amino acids 67 and 68 and its flanking amino acids abrogates the ability of OmpA to bind its receptor.

### Example 24

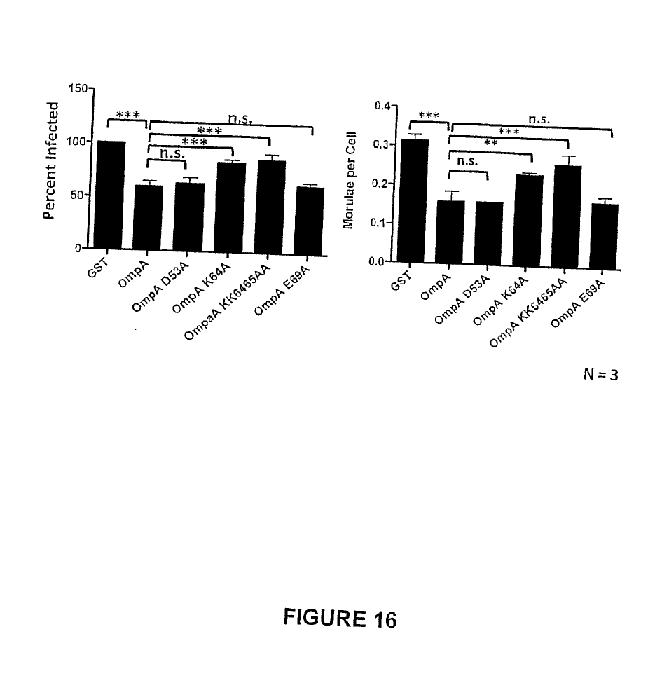

Alanine substitution experiments identify that amino acids within OmpA aa59-74 are important for infection. To identify specific amino acids that are important for OmpA to bind to and mediate infection of host cells, we performed PCR mutagenesis to create plasmids encoding GST-OmpA bearing single or double alanine substitutions at D53, K64, E69, K60A, K65, E72A, KK6065AA, KK6064AA, KKK606465AAA, or K64 and K65. The proteins were purified and added to mammalian host cells. Next, A. phagocytophilum bacteria were incubated with the host cells. GST-OmpA, GST-OmpAD53A, and GST-OmpA each significantly inhibited infection whereas GST alone did not (FIG. 16). The abilities of GST-OmpAK64A and GST-OmpAKK6465AA to antagonize infection were significantly less than that of GST-OmpA, which indicates that OmpA amino acids 64 and 65 are important for OmpA to properly bind to host cells and for recombinant OmpA to serve as a competitive agonist against A. phagocytophilum infection]

### Example 25

In silico modeling of OmpA interactions with its receptor. The tertiary structure for A. phagocytophilum OmpA was predicted using the PHYRE² (Protein Homology/analogy Recognition Engine, version 2.0) server (see the website at sbg.bio.ic.ac.uk/phyre2). The PHYRE² server predicts tertiary structures for protein sequences and threads the predicted structures on known crystal structures. The highest scoring model predicts that amino acids 59-74 to be part of a surface-exposed helix that would be available to interact with other molecules (data not shown). Indeed, when the autodock vina algorithm (http://vina.scripps.edu) is used to assess whether OmpA binds to its known receptor, sialic acid of the sialyl Lewis x antigen, the lowest free energy models predict that Lysine 64 interacts with sialic acid (data not shown).

### Example 26

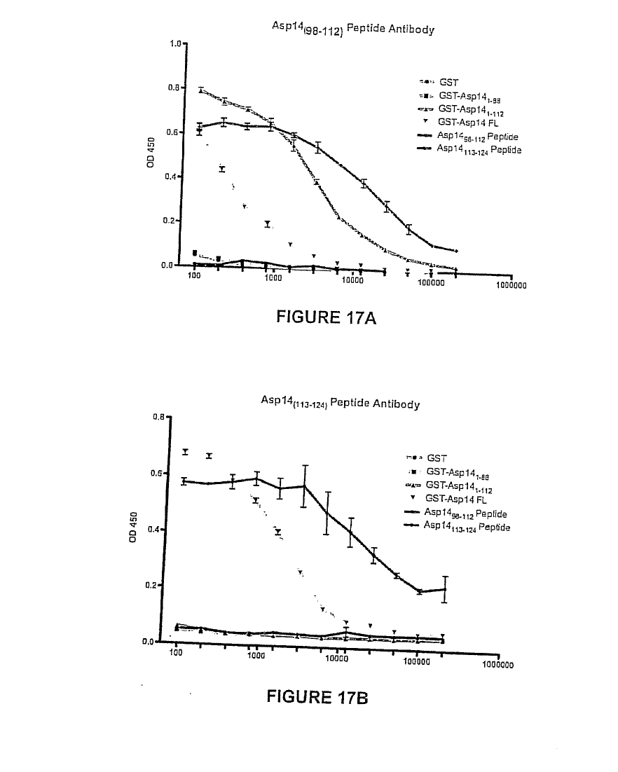

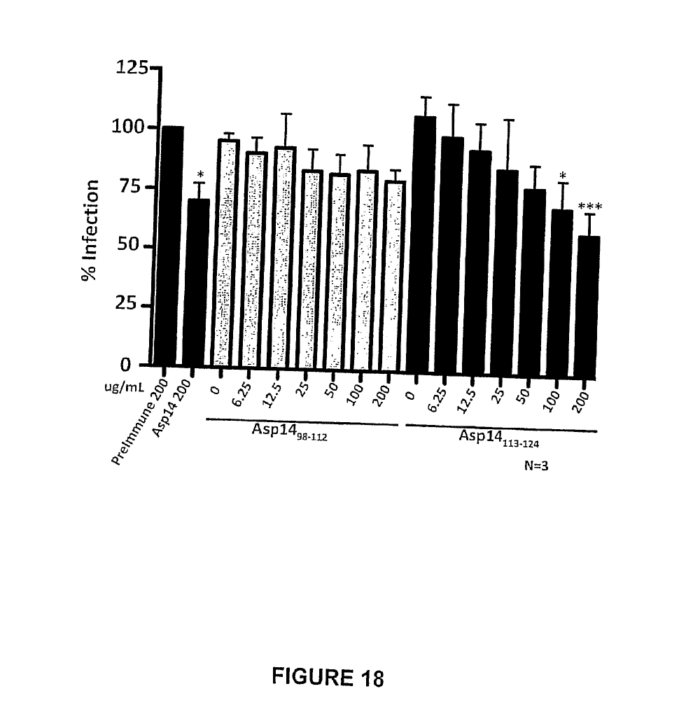

The Asp14 invasin domain lies within amino acids 113-124. The structure of Asp14 is not known and it cannot be predicted because it bears no semblance to any crystal structure. Next, we set out to identify the region of Asp14 that is important for infection. We knew that the Asp14 invasin domain lies within amino acids 101-124. We had rabbit antiserum raised against peptides corresponding to Asp14 amino acids 101-112 and 113-124. We confirmed by ELISA that each serum is specific for recombinant Asp14 and only the peptide against which it was raised (FIGS. 17A and B). Pretreating Aph with serum specific for Asp14₁₁₃₋₁₂₄ but not Asp14₁₀₁₋₁₁₂ significantly inhibited bacterial infection of host cells in vitro (FIG. 18).

### Example 27

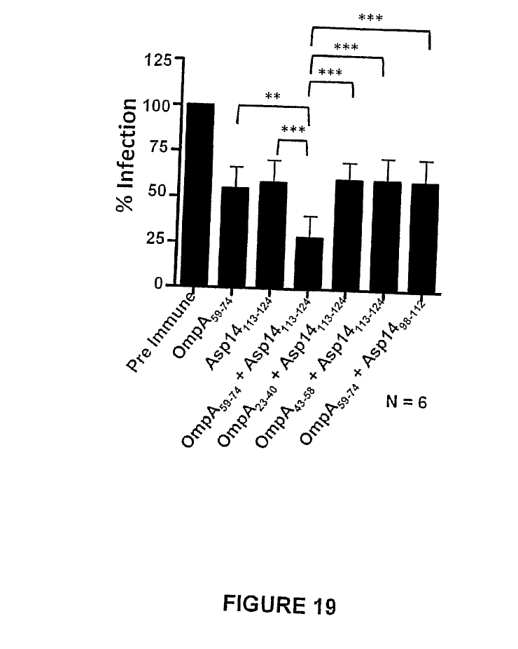

Treating Aph with antibodies targeting OmpA aa59-74 and Asp14 aa113-124 together pronouncedly inhibits infection of mammalian host cells Treating Aph organisms with anti-OmpA₅₉₋₇₄ or anti-Asp14₁₁₃₋₁₂₄ significantly inhibits infection of mammalian host cells in vitro (FIG. 19). Treating the bacteria with both anti-OmpA₅₉₋₇₄ and Asp14₁₁₃₋₁₂₄ even more pronouncedly inhibits infection.

### Example 28

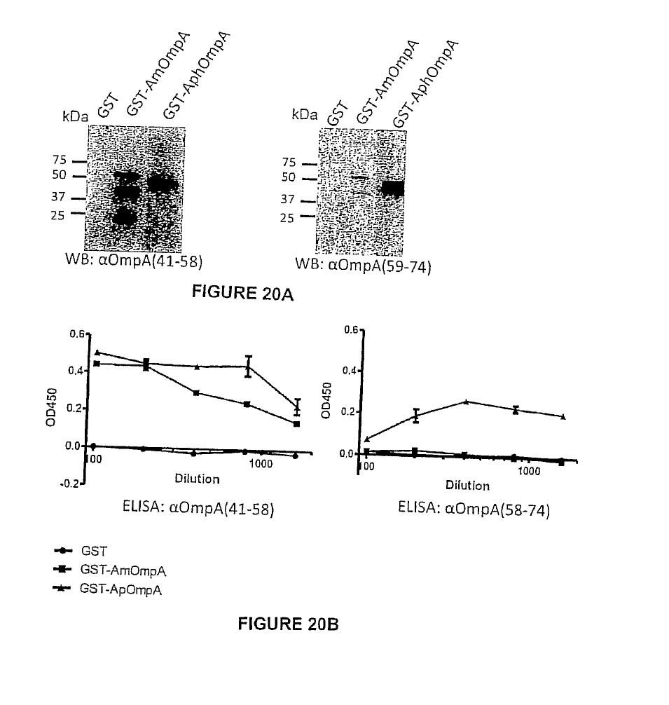

Aph OmpA and A. marginale OmpA share B-cell epitopes. A. marginale infects bovine red blood cells and costs the cattle industry hundreds of millions of dollars annually. A. marginale OmpA and Aph OmpA, though not identical, are very similar, including the region corresponding to Aph OmpA aa19-74 (SEQ ID NO:05). Therefore, a vaccine preparation that includes SEQ ID NO:05, alone or in combination with other sequences of the invention is also effective in providing protection against A. marginale infection. GST-tagged Aph OmpA, GST-tagged A. marginale OmpA (AM854), and GST alone were subjected to SDS-PAGE and transferred to nitrocellulose membrane. The blots were screened with serum from a cow that had been infected with A. marginale or with serum from a cow that had been immunized with purified A. marginale outer membrane proteins. Both sera recognized GST-tagged OmpA proteins not GST (data not shown), thereby demonstrating that OmpA proteins from Aph and A. marginale share B cell epitopes. Serum raised against Aph OmpA amino acids 41-58 or 59-74 recognize GST-A. marginale OmpA (AM854) in both Western blot (FIG. 20A) and ELISA (FIG. 20B).

### Example 28

Immunizing against OmpA and/or Asp14 protects mice from tick-mediated Aph infection. C3H/HeJ mice (female, 4-6 weeks of age) are immunized with 10 ug of GST-OmpA (full length), GST-OmpA₁₉₋₇₄, GST-Asp14; 10 ug each of GST-OmpA and GST-Asp14; or 10 ug each of GST-OmpA₁₉₋₇₄ and GST-Asp14 in Complete Freund's Adjuvant. At two and four weeks following the initial immunization, mice are boosted with the same amounts and combinations of each antigen in Incomplete Freund's Adjuvant.

Alternatively, C3H/HeJ mice are immunized with 50 ug of KLH-conjugated peptides corresponding to OmpA₂₃₋₄₀, OmpA₄₁₋₅₈, OmpA₅₉₋₇₄, Asp14₁₀₀₋₁₁₂, Asp14₁₁₃₋₁₂₄ and every possible combination thereof. The same adjuvants and immunization schedule as in the preceding paragraph may be followed.

Five days following the second boost, aliquots of serum from each mouse are tested via ELISA to confirm that a humoral immune response was mounted against OmpA, Asp14, and the respective portions thereof. At one week following the second boost, three Aph infected Ixodes scapularis ticks are placed on each mouse and allowed to feed for 48 hours to allow for transmission of the bacteria into the mice. On days 3, 8, and 12 post tick feeding, peripheral blood is collected. DNA isolated from the blood is subjected to quantitative PCR using primers targeting the Aph 16S rDNA and murine Beta-actin to determine the pathogen load in the peripheral blood (data not shown). This protocol is also useful when adjuvants suitable for innocculating dogs, humans, or other mammals are used for respective species.

### Example 29

Immunizing against OmpA and/or Asp14 protects mice from syringe inoculation of Aph infection C3H/HeJ mice (female, 4-6 weeks of age) are immunized with 10 ug of GST-OmpA (full length), GST-OmpA₁₉₋₇₄, GST-Asp14; 10 ug each of GST-OmpA and GST-Asp14; or 10 ug each of GST-OmpA₁₉₋₇₄ and GST-Asp14 in Complete Freund's Adjuvant. At two and four weeks following the initial immunization, mice are boosted with the same amounts and combinations of each antigen in Incomplete Freund's Adjuvant.

Alternatively, C3H/HeJ mice are immunized with 50 ug of KLH-conjugated peptides corresponding to OmpA₂₃₋₄₀, OmpA₄₁₋₅₈, OmpA₅₉₋₇₄, Asp14₁₀₀₋₁₁₂, Asp14₁₁₃₋₁₂₄ and every possible combination thereof. The same adjuvants and immunization schedule as in the preceding paragraph may be followed.

Five days following the second boost, aliquots of serum from each mouse are tested via ELISA to confirm that a humoral immune response was mounted against OmpA, Asp14, and the respective portions thereof. At one week following the second boost, each mouse is inoculated with either 100 ul of blood from an Aph infected SCID mouse that was confirmed to be infected or 100 ul of host cell free Aph bacteria recovered from tissue cell culture. On days 3, 8, and 12 post tick feeding, peripheral blood is collected. DNA isolated from the blood is subjected to quantitative PCR using primers targeting the Aph 16S rDNA and murine Beta-actin to determine the pathogen load in the peripheral blood (data not shown).

This protocol is also useful when adjuvants suitable for innocculating dogs, humans, or other mammals are used for respective species.

In summary, OmpA and Asp14 are the first two Aph surface proteins found to be critical for infection of mammalian cells. Expression of these proteins is induced in Aph during the tick bloodmeal and during the period in which humoral immune responses are stimulated in humans and mice. Embodiments of the invention are compositions comprising OmpA and/or Asp14 sequences and methods to prevent Aph infection of humans and animals by inducing an immune response that blocks one or more of the 3 critical stages of infection: (1) the initial colonization of neutrophils and/or endothelial cells that establishes infection; (2) the dissemination stage when infected peripherally circulating neutrophils are inhibited in their microbial killing capability; and (3) the infection of endothelial cells of heart and liver. A further embodiment provides compositions and methods for diagnosis of anaplasmosis and HGA.

### Example 30

Anaplasma marginale outer membrane protein A is an adhesin that recognizes sialylated and fucosylated glycans and functionally depends on an essential binding domain.

ABSTRACT. Anaplasma marginale causes bovine anaplasmosis, a debilitating and potentially fatal tick-borne infection of cattle. Because A. marginale is an obligate intracellular organism, its adhesins that mediate entry into host cells are essential for survival. Here, we demonstrate that A. marginale outer membrane protein A (AmOmpA; AM854) contributes to the invasion of mammalian and tick host cells. AmOmpA exhibits predicted structural homology to OmpA of A. phagocytophilum (ApOmpA), an adhesin that uses key lysine and glycine residues to interact with α2,3-sialylated and α1,3-fucosylated glycan receptors, including 6-sulfo-sialyl Lewis x. Antisera against AmOmpA or its predicted binding domain inhibits A. marginale infection of host cells. Residues G55 and K58 are contributory and K59 is essential for recombinant AmOmpA to bind to host cells. Enzymatic removal of α2,3-sialic acid and α1,3-fucose residues from host cell surfaces makes them less supportive of AmOmpA binding. AmOmpA is both an adhesin and an invasin, as coating inert beads with it confers adhesiveness and invasiveness. Recombinant forms of AmOmpA and ApOmpA competitively antagonize A. marginale infection of host cells, but a monoclonal antibody against 6-sulfo-sLe^{x} fails to inhibit AmOmpA adhesion and A. marginale infection. Thus, the two OmpA proteins bind related but structurally distinct receptors. This study provides a detailed understanding of AmOmpA function, identifies its essential residues that can be targeted by blocking antibody to reduce infection, and determines that it binds to one or more α2,3-sialylated and α1,3-fucosylated glycan receptors that are unique from those targeted by ApOmpA.

INTRODUCTION. Recombinant A. phagocytophilum OmpA (ApOmpA) binds to host cells, confers adhesiveness and invasiveness to inert beads, and acts as a competitive agonist to inhibit A. phagocytophilum infection in vitro, confirming that it alone is sufficient to mediate binding and uptake. ApOmpA functionally depends on a lysine and a glycine in its essential linear binding domain that interacts with α2,3-sialic acid and α1,3-fucose of the Lewis antigen receptors, sialyl Lewis x (sLe^{x}; NeuAcα2,3Galβ1,4[Fucα1,3]GlcNac) on myeloid cells and 6-sulfo-sLe^{x} (NeuAcα2,3Galβ1-4[Fucα1,3]HSO₃3,6GlcNac) on endothelial cells. Antibodies raised against full-length ApOmpA or its 16-residue binding domain inhibit A. phagocytophilum infection of host cells. Likewise, antibodies against E. chaffeensis OmpA inhibit ehrlichial infection in vitro.

In this study, we demonstrate that A. marginale OmpA (AmOmpA) is an adhesin that contributes to A. marginale infection of mammalian and tick host cells. The adhesin capability of AmOmpA depends on specific lysine and glycine residues located within an essential binding domain, the position of which is predicted to be structurally conserved with that of ApOmpA. It recognizes an α2,3-sialylated and α1,3-fucosylated glycan on endothelial cells that is not 6-sulfo-sLe^{x}. Collectively, these data reveal the pathobiological role of AmOmpA, identify its essential region that can be targeted by antibodies to inhibit infection, and underscore the conserved pathobiological importance of OmpA proteins to Anaplasma and Ehrlichia spp.

MATERIALS AND METHODS. Cultivation of uninfected and infected A. marginale infected host cell lines. Uninfected and A. marginale (St. Manes strain)-infected RF/6A rhesus monkey choroidal endothelial cells (CRL-1780, American Type Culture Collections, Manassas, Va.), and Ixodes scapularis embryonic ISE6 cells were cultured.

Site directed mutagenesis and recombinant protein production. AmOmpA nucleotides 60 to 708, which encode residues 21 to 236 lacking the signal sequence (mature AmOmpA), were PCR amplified using primers containing the BamHI and Not1 restriction sites (5′-GATCGGATCCCTTTTCAGCAAGGAAAAGGTCGGGATG-3′ (SEQ ID NO: 73) and 5′-ATCGGCGGCCGCCTATTCAGGCGCGACCACTCC-3′ (SEQ ID NO: 74) [boldface indicates extra nucleotides upstream of restriction sites; restriction sites are underlined]). The sequence integrity of the resulting PCR product was verified, after which it was digested and ligated into pGEX4T1 (GE Healthcare Bio-Sciences, Pittsburgh, Pa.) that had been digested with BamH1 and Not1. GST-AmOmpA was expressed and purified by glutathione Sepharose affinity chromatography. AmOmpA genes encoding proteins with D47, K54, G55, K58, and/or K59 replaced with alanine were synthesized. Plasmids encoding His-tagged mature wild type AmOmpA and site-directed versions thereof were generated by amplifying wild type and mutant AmOmpA sequences using primers 5′-GACGACGACAAAATGCTTTTCAGCAAGGAAAA-3′ (SEQ ID NO: 75) and 5′-GAGGAGAAGCCCGGTTACTATTCAGGCGCGA-3′ (SEQ ID NO: 76) [boldface indicates ligase-independent cloning (LIC) tails] and annealing the amplicons into the pET46 Ek/LIC vector (Novagen, EMD Millipore, Darmstadt, Del.) per the manufacturer's instructions. His-OmpA proteins were expressed and purified by immobilized metal-affinity chromatography. GST-ApOmpA, His-ApOmpA, His-OtOmpA (Orientia tsutsugamushi OmpA) have been previously described.

Antibodies, reagents, Western blotting, and enzyme-linked immunosorbent assay (ELISA). His-AmOmpA was used to immunize rats and the resulting antiserum was collected. New England Peptide (Garner, Mass.) generated serum against the AmOmpA putative binding domain as follows. A peptide corresponding to AmOmpA residues 50 to 67 (AmOmpA₅₀₋₆₇) was synthesized, conjugated to keyhole limpet hemocyanin, used to immunize rabbits, and the resulting serum was affinity-purified. Antiserum against A. phagocytophilum OmpA₅₉₋₇₄ has been previously described. Each antiserum's specificity was determined by ELISA using GST, GST-AmOmpA, GST-ApOmpA, and AmOmpA₅₀₋₆₇ as immobilized antigens and the TMB substrate kit (Thermo Scientific, Waltham, Mass.) following the manufacturer's instructions or by Western blot. Each antiserum's concentration was determined using the Bradford assay. Fragments of antibody binding (Fab) of mouse anti-AmOmpA and rabbit anti-AmOmpA₅₀₋₆₇ were generated using the Fab Preparation Kit (Pierce, Rockford, Ill.). Fab concentrations were determined based on absorbance at 280 nm. Monoclonal antibody AnaF16C1, which recognizes A. marginale major surface protein 5 and was used to detect the bacterium in indirect immunofluorescence microscopy assays, was provided. sLe^{x} antibodies CSLEX1 (BD Biosciences, San Jose, Calif.) and KM93 (Millipore, Darmstadt, Del.) were obtained commercially. 6-sulfo-sLe^{x} antibody, G72, has been described previously. Alexa Fluor 488-conjugated anti-His tag secondary antibody and Alexa Fluor 488-conjugated streptavidin were obtained from Invitrogen (Carlsbad, Calif.). Biotinylated AAL and MAL II were obtained from Vector Labs (Burlingame, Calif.). Glycosidases used in this study were α2,3/6-sialidase (Sigma-Aldrich, St. Louis, Mo.) and α1,3/4-fucosidase (Clontech, Mountain View, Calif.). Lectins and glycosidases were used as previously described.

Molecular modeling of AmOmpA. To obtain a putative tertiary AmOmpA protein structure, the mature AmOmpA sequence was threaded onto solved crystal structures of proteins with similar sequences using the PHYRE2 server. Amino acids 29 to 154 (58% of the mature AmOmpA sequence) were modeled with greater than 90% confidence to known structures for similar proteins (Protein Data Bank [PDB] files 2aiz [Haemophilus influenzae OmpP6 peptidoglycan associated lipoprotein (PAL)], 4g4x [Acinetobacter baumannii PAL], 4b5c [Burkholderia pseudomallei PAL], 2hqs [Escherichia coli PAL], and 2126 [OmpA-like domain of Mycobacterium tuberculosis ArfA]). The remainder of the protein lacked sufficient homology to any experimentally derived structure, but could be modeled using the Poing method, which was performed as part of the PHYRE2 analyses. To generate the overlay, PHYRE2 models from mature ApOmpA and mature AmOmpA were threaded onto each other using PyMOL. Mature AmOmpA surface electrostatic values were calculated using the PyMol adaptive Poisson-Boltzman solver (APBS) plugin for PyMOL.

Binding of recombinant proteins to host cells. RF/6A cells were incubated with 4 μM of recombinant His-tagged AmOmpA proteins in culture media for 1 h in a 37° C. incubator supplemented with 5% CO₂ and a humidified atmosphere. Binding was assessed via flow cytometry or immunofluorescence microscopy. In some cases, cells were pretreated with α2,3-sialidase (5 μg/mL), α1,3/4-fucosidase (10 μU/mL), CSLEX1 (10 μg/mL), KM93 (10 μg/mL), or G72 (10 μg/mL) prior to the addition of AmOmpA.

Competitive inhibition of A. marginale infection. A. marginale infected RF/6A cells that were >90% infected and beginning to lyse were sonicated to destroy host cells and RC organisms, but leave DC organisms intact. Cellular debris was removed by two successive 5-min centrifugation steps at 1000 g. A. marginale DC bacteria were pelleted by centrifugation at 5000 g for 10 min. For competitive inhibition assays using antiserum and RF/6A cells, A. marginale DC organisms were incubated with AmOmpA antiserum (200 μg/mL), AmOmpA₅₀₋₆₇ antiserum (200 μg/mL), or Fab fragments thereof (200 μg/mL) for 1 h, after which bacteria were incubated with host cells at a multiplicity of infection (MOI) of approximately 1 in the continued presence of antibodies for 2 h. Pre-immune rat or rabbit serum (200 μg/mL) was used as a negative control. Unbound bacteria were removed and infection was allowed to proceed for 48 h. To determine if recombinant OmpA proteins could antagonize A. marginale infection, RF/6A cells were incubated with GST-AmOmpA, GST-ApOmpA, or GST alone (4 μM) for 1 h, after which A. marginale DC organisms were added and incubated with the host cells in the continued presence of recombinant protein for 2 h. Unbound bacteria and proteins were removed and the infection was allowed to proceed for 48 h. Experiments that assessed if antibodies targeting AmOmpA or recombinant OmpA proteins could inhibit A. marginale infection of ISE6 cells were performed identically as those just described except that A. marginale organisms were incubated with ISE6 cells for 5 h before unbound bacteria were removed, the infection was allowed to proceed for 72 h, and the MOI achieved was approximately 1.7. At the endpoint of each experiment, cells were analyzed by spinning-disk confocal microscopy to determine the percentage of infected cells and number of AmVs per cell.

OmpA coated bead uptake assay. His-AmOmpA was conjugated to red fluorescent sulfate-modified 1.0-μm diameter microfluospheres (Life Technologies, Carlsbad, Calif.). Coated and uncoated beads were incubated with RF/6A cells in culture medium at a bead-to-cell ratio of 500:1. Binding and internalization of the beads were assessed by spinning-disk confocal microscopy.

Statistical analysis. The Student's t-test or one-way analysis of variance (ANOVA) was performed using the Prism 5.0 software package (Graphpad, San Diego, Calif.). Statistical significance was set to P <0.05.

### Results

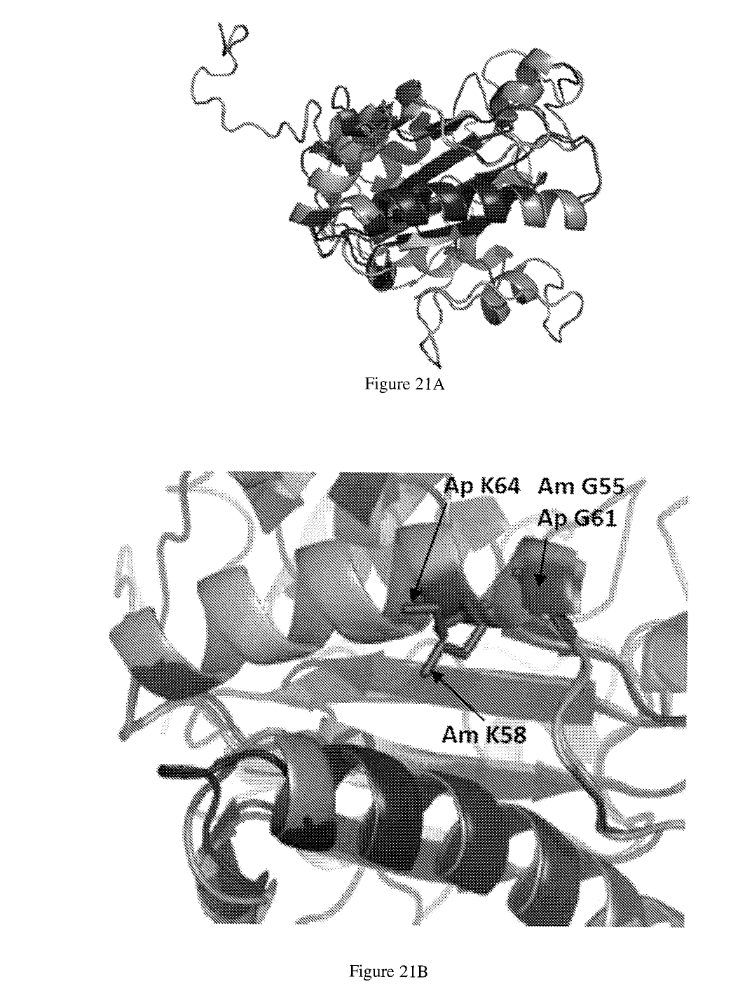

Molecular modeling reveals high predicted structural homology between AmOmpA and ApOmpA and delineates a putative binding domain. An alignment of ApOmpA and AmOmpA revealed that the two exhibit 52.33% sequence identity. Notably, one particular stretch where the two proteins exhibit considerable identity occurs between ApOmpA residues 59 to 74 (ApOmpA₅₉₋₇₄; L₅₉KGPGKKVILELVEQL₇₄; SEQ ID NO: 06), which forms the essential binding domain, and AmOmpA residues 53 to 68 (AmOmpA₅₃₋₆₈; I₅₃KGSGKKVLLGLVERM₆₈; SEQ ID NO: 77; identical and similar residues between the two peptides are denoted by bold and underlined text, respectively). In our preceding study, molecular modeling of ApOmpA predicted that residues 59 to 74 form a surface-exposed alpha helix of which G61 and K64 help form a binding pocket that interacts with Lewis antigen receptors. This model proved highly useful for directing experiments that validated the functional essentiality of ApOmpA G61 and K64. Therefore, as a first step in assessing the potential adhesin role of AmOmpA, molecular modeling of amino acids 19 to 236 (excluding the signal sequence) was performed using the PHYRE2 recognition server, which predicts three-dimensional structures for protein sequences and threads the predicted models on known crystal structures. Threading the AmOmpA and ApOmpA tertiary models onto each other using PyMOL revealed that the two are very structurally similar and that the relative positions of the AmOmpA₅₃₋₆₈ and ApOmpA₅₉₋₇₄ alpha helices overlap (FIGS. 21, A and B). Moreover, the predicted tertiary locations of AmOmpA G55 and K58 overlay perfectly with ApOmpA G61 and K64, respectively (FIG. 21B). A space filling model of AmOmpA indicated that G55, K58, and flanking residues might form a binding pocket that is structurally analogous to that predicted for ApOmpA. ApOmpA and other microbial proteins that interact with sLe^{x} do so at cationic surface patches. Consistent with this trend, using the APBS plugin for PyMOL to calculate AmOmpA surface electrostatic values predicted that amino acids 19 to 67, which contains the region that is homologous to the sLe^{x}/6-sulfo-sLe^{x} binding domain of ApOmpA, have an overall cationic surface charge. These data suggest that AmOmpA functions as an adhesin and that key amino acids within the stretch comprised by residues 53 to 68 are functionally essential.

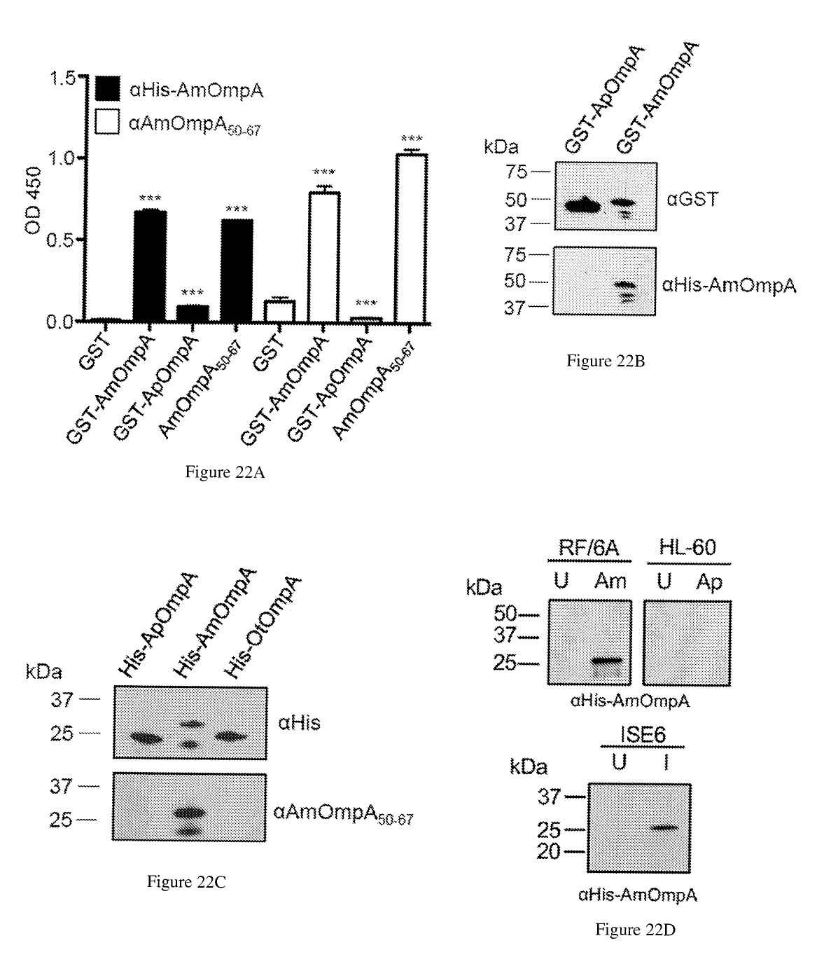

Antisera raised against AmOmpA and its putative binding domain inhibit infection of mammalian host cells. Antisera against His-tagged mature AmOmpA and a peptide corresponding to its putative binding domain was generated. For the binding domain peptide, one comprising residues 50 to 67 was selected because it contains all of the residues that are likely to be critical for function, as described below, and has a higher Jameson-Wolfe antigenicity index score than one corresponding to residues 53 to 68. Both antisera recognized recombinant versions of AmOmpA, AmOmpA₅₀₋₆₇, and exhibited no to minimal cross-reactivity via Western blot and ELISA with GST alone, recombinant ApOmpA proteins, or a His-tagged version of OmpA from Orientia tsutsugamushi, an obligate intracellular bacterial pathogen that is in the order Rickettsiales with Anaplasma spp. (FIG. 22, A-C). Screening A. marginale infected RF/6A endothelial and tick embryonic ISE6 cells and A. phagocytophilum infected promyelocytic HL-60 cells with anti-AmOmpA detected a band of the expected size for AmOmpA only in lysates of A. marginale infected cells (FIG. 22D). Thus, AmOmpA and AmOmpA₅₀₋₆₇ antisera exclusively recognize their target antigens. An additional observation gleaned from these data is that, while A. phagocytophilum expresses OmpA during infection of mammalian but not tick cells, A. marginale expresses OmpA during infection of both host cell types.

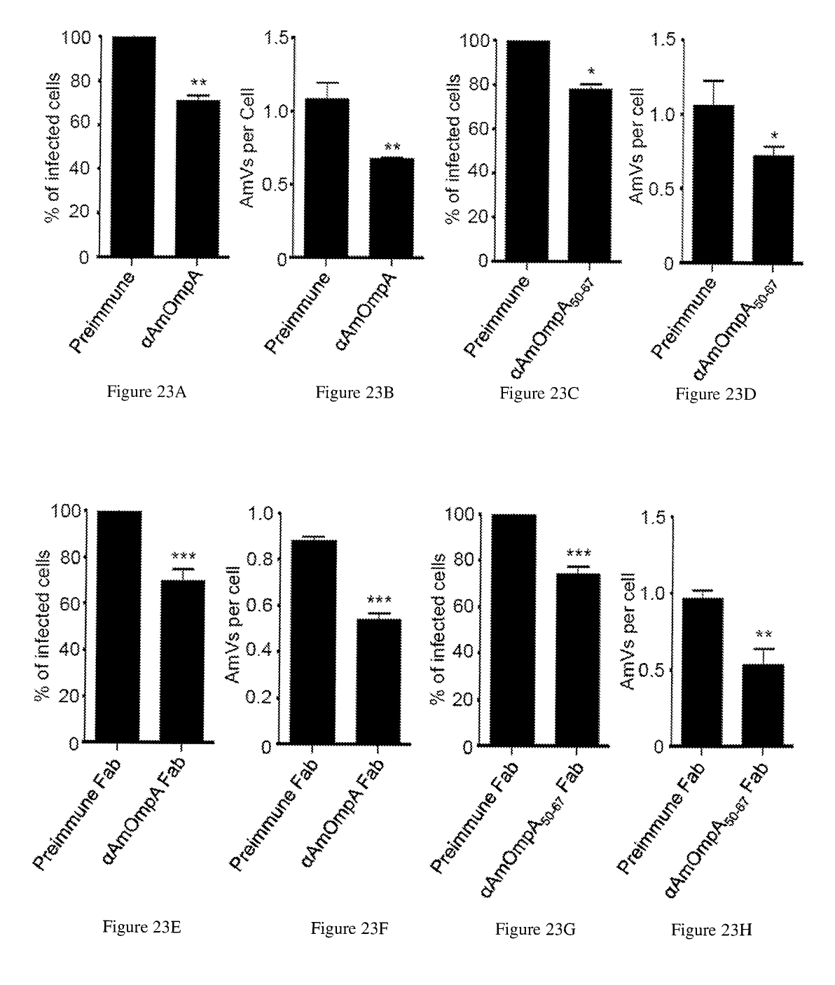

Next, the abilities of both antisera to inhibit A. marginale infection of mammalian host cells were evaluated. A. marginale DC organisms were treated with heat-inactivated AmOmpA or AmOmpA₅₀₋₆₇ antiserum prior to incubation with RF/6A cells. After 48 h, infection was assessed using immunofluorescence microscopy. Each antiserum reduced the percentage of infected cells by approximately 25% and decreased the number of AmVs per cells by approximately 40%, whereas preimmune serum had no effect (FIG. 23, A-D). To ensure that the blocking effects achieved were specific and not due to steric hindrance, the experiments were repeated using fragment antigen binding (Fab fragment) portions of anti-AmOmpA and anti-AmOmpA₅₀₋₆₇. Blocking achieved with the Fab fragments was identical to that achieved with intact antibodies (FIG. 23, E-H). These data indicate that AmOmpA contributes to A. marginale infection of mammalian host cells. Moreover, the high similarity of the inhibitory effects achieved by anti-AmOmpA and anti-AmOmpA₅₀₋₆₇ supports that residues within 50 to 67 are important for AmOmpA-mediated infection.

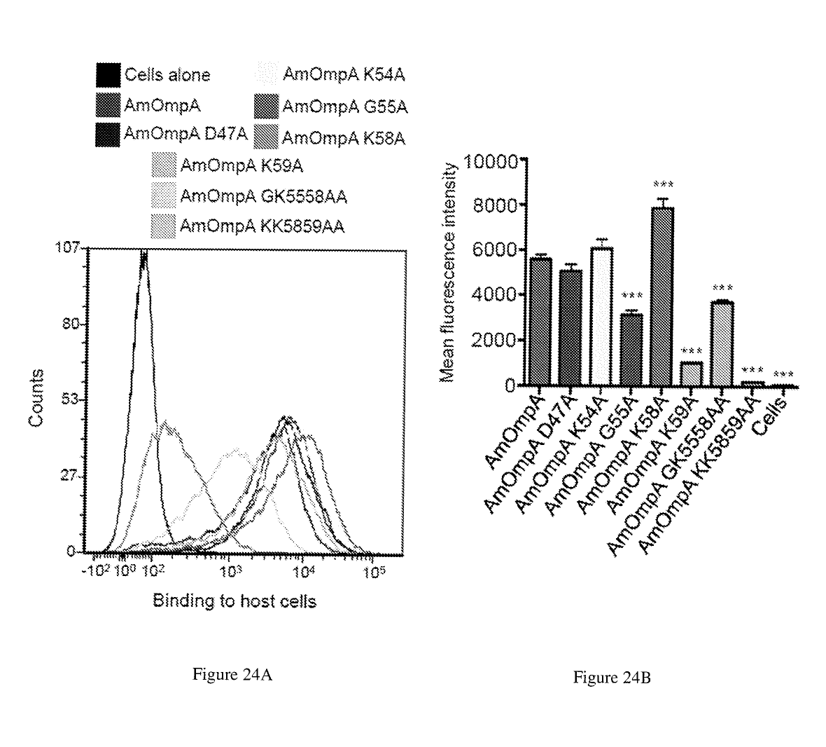

G55, K58, and K59 are critical for recombinant AmOmpA to bind to mammalian host cells. To determine if AmOmpA exhibits adhesin activity and, if so, to define the importance of individual amino acid residues within the binding domain to such activity, His-tagged AmOmpA and versions thereof in which specific residues were mutated to alanine were assessed for the ability to bind to RF/6A cells using flow cytometry. ApOmpA binding domain residues G61 and K64, but not other binding domain residues are functionally essential. Therefore, AmOmpA G55 and K58 were prioritized for substitution because they align both sequentially and in relative position in the predicted tertiary structure with ApOmpA G61 and K64. K54 and and K59 were also replaced with alanine since they immediately flank G55 and K58. D47 was substituted as a negative control because it lies outside the AmOmpA binding domain and corresponds to ApOmpA D53, which was previously shown to be functionally irrelevant. As expected, both His-AmOmpA and His-AmOmpA_{D47A} bound to host cells (FIG. 24A-B). K54 is dispensable for AmOmpA function, as His-AmOmpA_{K54A} was uncompromised in its ability to bind to host cells. His-tagged AmOmpA_{G55A} and AmOmpA_{K59} displayed modest and considerably more pronounced reductions in binding. Substituting K58 alone led to an increase in binding, and replacing it together with G55 did not further reduce binding compared to substituting G55 alone. However, replacing K58 together with K59 abolished binding. Overall, these observations demonstrate that AmOmpA adhesin function critically relies on G55, K58, and K59.

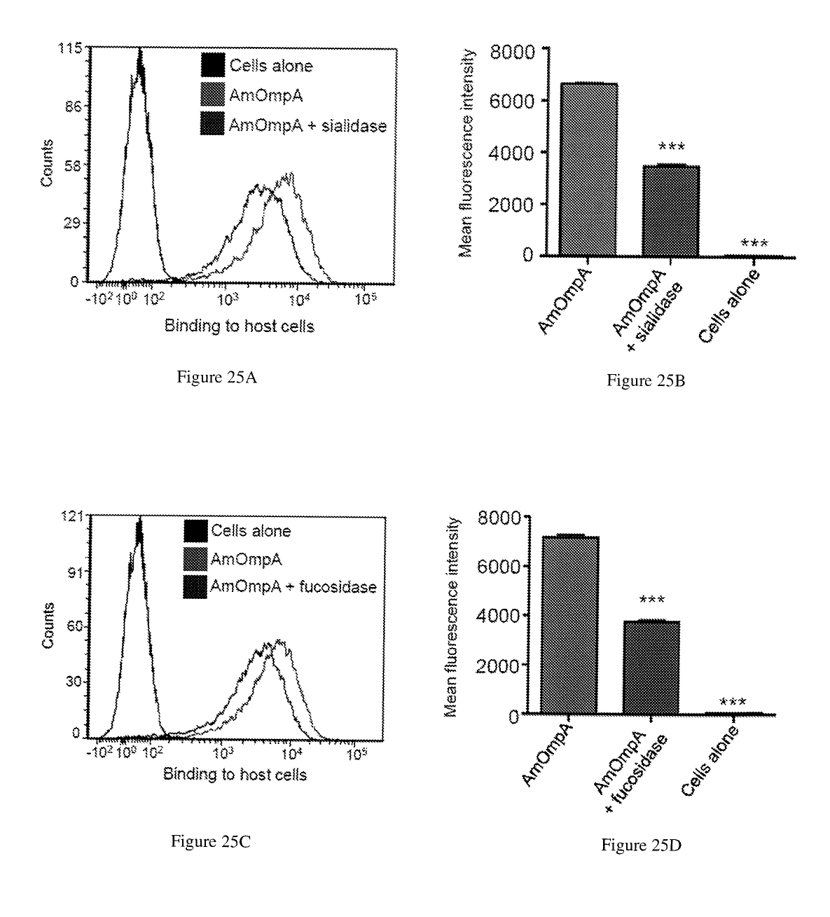

AmOmpA interacts with sialic acid and fucose on mammalian host cells. Consistent with it being an adhesin that interacts with α2,3-sialylated and α1,3-fucosylated receptors on mammalian host cells, binding of recombinant ApOmpA to cell surfaces from which either sugar residue has been enzymatically removed is significantly reduced. To determine if AmOmpA binds to α2,3-sialic acid or α1,3-fucose, His-tagged AmOmpA was incubated with RF/6A cells that had been treated with α2,3/6-sialidase or α1,3/4-fucosidase, respectively, and binding was assessed by immunofluorescence microscopy and flow cytometry. To verify the efficacy of the glycosidases, treated and untreated cells were screened with lectins that recognize fucose and sialic acid residues that are in the specific linkages of interest. AAL (Aleuria aurantia lectin) recognizes fucose residues that are in α1,3- and α1,6-linkages with N-acetylglucosamine. MAL II (Maackia amurensis lectin II) recognizes sialic acid residues that are in α2,3-linkages with galactose. Fucosidase treatment abolished binding of AAL, but not MAL II. Conversely, sialidase treatment prevented binding of MAUI, but not AAL. Thus, the glycosidases effectively and specifically enzymatically removed their target sugar residues. His-AmOmpA binding to sialidase- and fucosidase-treated cells was similarly reduced compared to vehicle control treated cells (FIG. 25, A-D). Thus, AmOmpA utilizes both α2,3-sialic acid and α1,3-fucose for optimal adhesion to host cells.

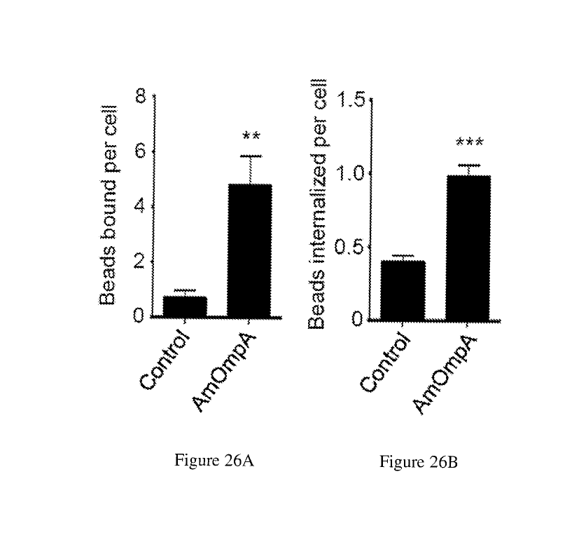

AmOmpA-coated beads bind to and are internalized by endothelial cells. The ability of His-AmOmpA to bind to host cells suggests that it exhibits adhesin function. Whether it also functions as an invasin is unknown. As a complementary approach to confirm its adhesin activity and to assess its capacity to function as an invasin, the ability of His-AmOmpA to confer adhesiveness and invasiveness to inert particles was assessed. His-AmOmpA was conjugated to red fluorescent microspheres that were 1.0 μm in diameter, which approximates the diameter of a typical A. marginale DC organism (0.8±0.2 μm). Non-phagocytic RF/6A endothelial cells were incubated with recombinant AmOmpA-coated or non-coated control beads and screened with AmOmpA antibody to determine the numbers of beads bound per cell. To measure bead internalization, the cells were incubated for an additional 7 h and trypsin was used to remove non-internalized beads prior to screening. Immunofluorescence microscopy confirmed that significantly more AmOmpA coated beads bound to and were internalized by RF/6A cells compared to non-coated control beads (FIG. 26A-B), thereby demonstrating that AmOmpA has the capacity to act as both an adhesin and invasin.

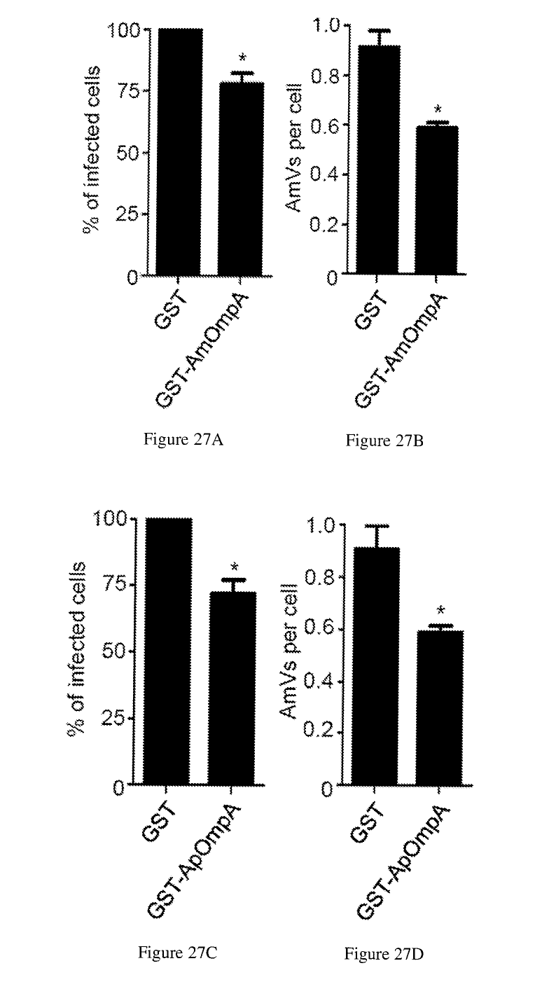

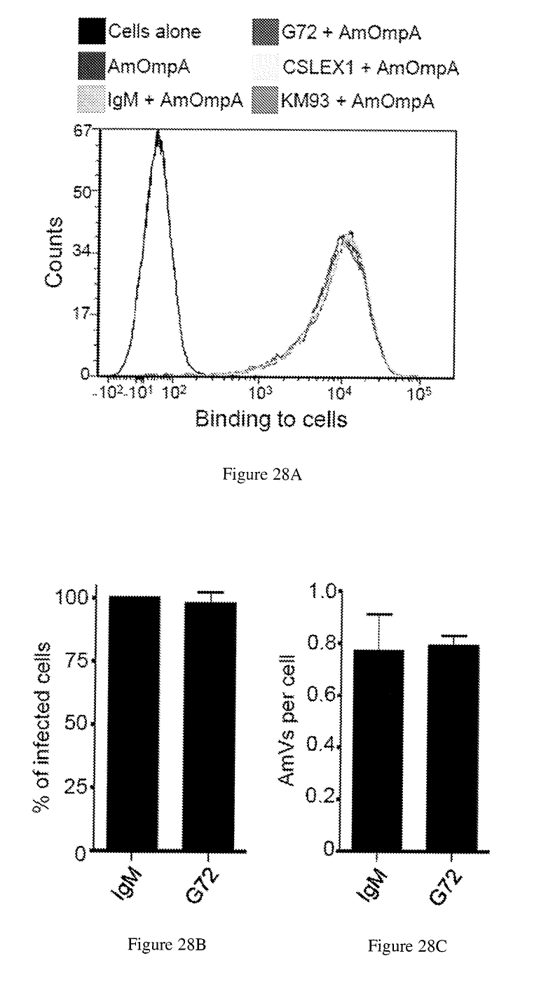

AmOmpA and ApOmpA recognize different, but structurally similar receptors on endothelial cells. Recombinant ApOmpA binding to the 6-sulfo-sLe^{x} receptor competitively inhibits A. phagocytophilum infection of RF/6A cells. Because AmOmpA binding to RF/6A cells involves recognition of α2,3-sialic acid and α1,3-fucose, because AmOmpA and ApOmpA each bind to RF/6A cells, and because of the homologies between the two proteins' binding domains, we rationalized that they might recognize the same or structurally similar receptors on endothelial cells. If so, then recombinant forms of AmOmpA and ApOmpA should competitively antagonize A. marginale infection of RF/6A cells to comparable degrees. Indeed, preincubating the host cells with GST-tagged AmOmpA and ApOmpA led to similar reductions in the percentage of infected cells and the mean number of AmVs per cell (FIG. 27A-D). To determine if AmOmpA interacts with 6-sulfo-sLe^{x} on RF/6A cells, His-AmOmpA binding to the host cells treated with the 6-sulfo-sLe^{x}-specific monoclonal antibody, G72, was assessed. This antibody was previously confirmed to bind to RF/6A cell surfaces and thereby inhibit recombinant ApOmpA adhesion. Monoclonal antibodies CSLEX1 and KM93 that recognize sLe^{x}, which is poorly expressed on RF/6A cells, and IgM served as negative and isotype controls, respectively. None of the antibodies inhibited His-AmOmpA binding (FIG. 28A). Likewise, G72 was ineffective at inhibiting A. marginale infection of RF/6A cells (FIGS. 28, B and C). Taken together, these data and the results presented above indicate that both recombinant AmOmpA and native AmOmpA on the A. marginale surface recognize an α2,3-sialylated and α1,3-fucosylated receptor on endothelial cells that is distinct from the ApOmpA endothelial receptor, 6-sulfo-sLe^{x}.

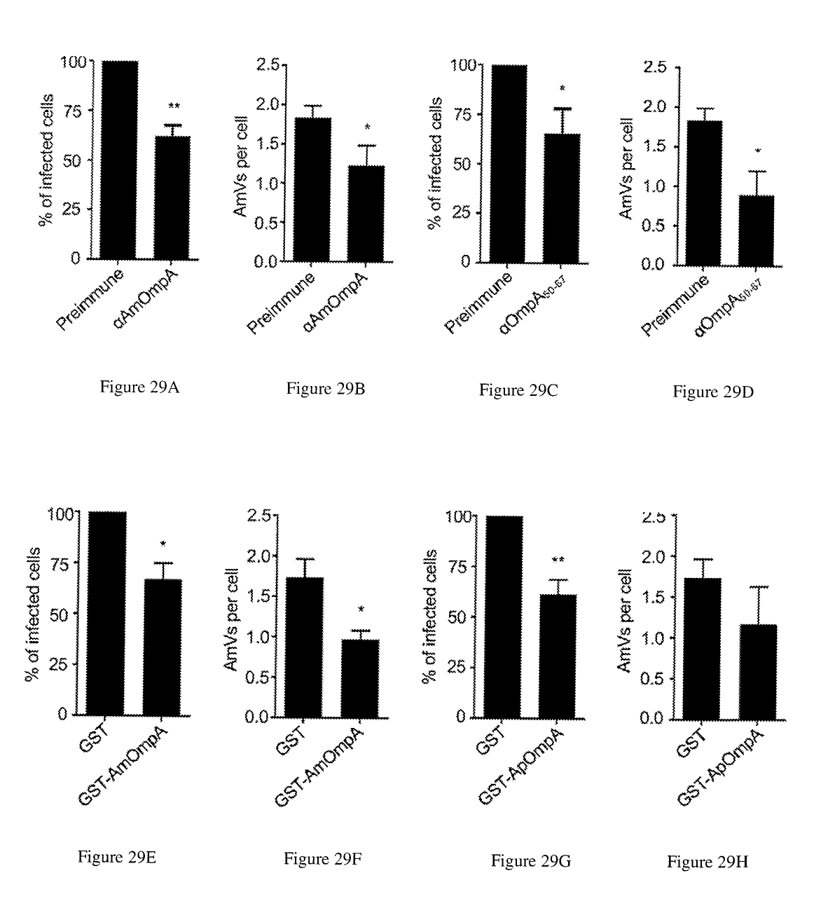

AmOmpA contributes to A. marginale infection of tick cells in a manner that is dependent on residues 50 to 67. Because A. marginale also infects tick cells, the relevance of AmOmpA to A. marginale infection of ISE6 cells was examined. Treating DC organisms with heat-inactivated AmOmpA or AmOmpA₅₀₋₆₇ antiserum prior to incubation with ISE6 cells significantly reduced the percentage of infected cells and number of AmVs per cell to comparable degrees as observed for RF/6A cells (FIG. 29, A to D). Thus, AmOmpA contributes to A. marginale infection of tick cells and requires amino acids 50 to 67 to optimally do so. Also, GST-tagged AmOmpA and ApOmpA competitively antagonized A. marginale infection of ISE6 cells (FIG. 29, E to H), suggesting that both recognize either the same or a structurally similar receptor on tick cells that A. marginale engages as part of its infection strategy. Sialic acids are rare in invertebrates and have not been detected in I. scapularis, but α1,3-fucose residues are important for A. phagocytophilum to colonize these ticks. An evaluation of whether AmOmpA binding involves recognition of α1,3- or α1,4-fucose residues ISE6 cells could not be attempted because α1,3/4-fucosidase treatment failed to reduce AAL binding, indicating that ISE6 cell surfaces have an abundance of fucose residues that exist in α1,6 or other linkages that would not be cleaved by α1,3/4-fucosidase.

### Discussion

Identifying A. marginale adhesins, delineating their functional domains, and determining the host cell determinants to which they bind not only will augment fundamental understanding of A. marginale pathobiology, but also could benefit development of novel approaches for protecting against bovine anaplasmosis. Herein, we determined that AmOmpA contributes to A. marginale invasion of mammalian host cells. Its binding domain lies within amino acids 50 to 67, as AmOmpA₅₀₋₆₇ antibody inhibited bacterial infection of RF/6A cells. This region is homologous both in sequence and predicted structural location to the ApOmpA binding domain. Moreover, the positions of two of the three AmOmpA amino acids determined to be essential for adhesin function, G55 and K58, are identical to those of ApOmpA functionally essential residues, G61 and K64. Whereas AmOmpA K59 is important for function, analogous ApOmpA K65 is not, which may at least partially account for the disparity between the two proteins' abilities to recognize 6-sulfo-sLe^{x} versus an as yet identified α2,3-sialylated and α1,3-fucosylated glycan. G55 and K59 are conserved among OmpA proteins of Anaplasma spp., while K58 is conserved among those of Anaplasma and Ehrlichia spp. Replacing only K58 with alanine resulted in no loss of AmOmpA function. However, the importance of K58 became apparent when it and G55 or K59 were both substituted with alanine, as AmOmpA GK5558AA and KK5859AA binding to host cells was nearly abolished. Given its demonstrated role in AmOmpA and ApOmpA function, K58 likely contributes to the adhesin capabilities of all Anaplasma and Ehrlichia spp. OmpA proteins. Our findings presented herein together with a previous report that E. chaffeensis OmpA contributes to infection of monocytic cells suggest that ehrlichial OmpA proteins are also adhesins that contribute to cellular invasion and do so by recognizing sialylated and fucosylated glycans in a manner that involves the conserved lysine.

AmOmpA G55, K58, and K59 are predicted to form a cationic binding pocket. This is likely critical for OmpA to recognize negatively charged fucose and sialic acid, as positively charged patches of numerous microbial sialic acid binding proteins have been shown to be important for receptor binding. Indeed, recombinant AmOmpA proteins in which G55 or K59 had been substituted with alanine were modestly and pronouncedly compromised, respectively, in their abilities to bind to host cells. Recombinant AmOmpA in which K58 and K59 were both mutated to alanine was devoid of adhesin capability. This additive reduction in binding is presumably due to the large net loss in positive charge in the binding domain.

Recombinant ApOmpA and AmOmpA competitively antagonize A. marginale infection of RF/6A cells to comparable degrees, and AmOmpA binding to cells from which α2,3-sialic acid or α1,3-fucose have been removed is compromised. Together, these findings indicate that one or more sialylated and fucosylated glycans recognized by AmOmpA are important for A. marginale cellular invasion. However, our hypothesis that the AmOmpA endothelial cell receptor was the same as that bound by ApOmpA, 6-sulfo-sLe^{x}, proved incorrect. 6-sulfo-sLe^{x} antibody G72 did not affect recombinant AmOmpA binding to or A. marginale infection of host cells, suggesting that AmOmpA engages a distinct sialylated and fucosylated glycan. Support for this premise comes from the fact that although ApOmpA preferentially recognizes 6-sulfo-sLe^{x}, G72 inhibits but does abrogate recombinant ApOmpA binding to host cells. This indicates that ApOmpA is also able to recognize other sialylated and fucosylated glycans, potentially the AmOmpA primary endothelial cell receptor, which could explain why recombinant ApOmpA but not G72 inhibits recombinant AmOmpA binding to RF/6A cells. Without being bound by theory, the differential preference of the two OmpA proteins for similar but distinct receptors could be related to the tropism of A. phagocytophilum and A. marginale for neutrophils and erythrocytes, respectively. Given that ApOmpA binds distinct but structurally related receptors on myeloid and endothelial cells, the same could be true of the receptors that AmOmpA binds on erythrocytes and endothelial cells. A second possibility is that AmOmpA binds to a receptor that is shared by red blood and endothelial cells.

AmOmpA by itself functions as both an adhesin and an invasin, as demonstrated by the ability of His-AmOmpA to confer adhesiveness and invasiveness to inert beads. However, by itself it does so inefficiently, as only 25% of the bound His-AmOmpA beads internalized. Similarly, competitively inhibiting A. marginale infection using recombinant AmOmpA or antiserum targeting AmOmpA or AmOmpA₅₀₋₆₇ reduces infection by only 25%. Because A. marginale uses multiple surface proteins to mediate binding and entry, compensatory actions of other adhesins likely facilitate infection when AmOmpA is blocked.

ISE6 tick cell culture is an acceptable model for studying A. marginale infection of tick cells. Using this cell line, we discovered that AmOmpA is also important for A. marginale infection of tick cells and that the same AmOmpA₅₀₋₆₇ domain that is key for the bacterium to optimally invade RF/6A cells is also critical for tick cell infection. This finding combined with the observation that recombinant AmOmpA and ApOmpA competitively antagonize A. marginale infection of tick cells to comparable degrees suggests that AmOmpA recognizes the same or a structurally similar receptor on the tick cell surface. A notable discrepancy between AmOmpA and ApOmpA is that the former is expressed during growth in ISE6 cells, while the latter is not. Why then does recombinant ApOmpA bind to and antagonize A. marginale infection of ISE6 cells? The answer might lie in the fact that A. phagocytophilum expresses ApOmpA while in a mammalian host and would therefore be present on the bacterium's surface when introduced into the tick by the acquisition bloodmeal. As A. phagocytophilum requires an α1,3-fucosylated receptor to colonize its tick vector, ApOmpA could be linked to this ability.

We claim:

1. An immunogenic composition including one or more isolated polypeptides in a vehicle or carrier suitable for administration to a subject, wherein at least one of said one or more polypeptides consists of 5 to 19 consecutive residues of SEQ ID NO:84 including SEQ ID NO: 85, wherein said at least one of said one or more polypeptides does not consist of 16 consecutive residues of SEQ ID NO:84.

2. The immunogenic composition of claim 1, wherein said at least one of said one or more polypeptides is SEQ ID NO:81 or SEQ ID NO:85.

3. The immunogenic composition of claim 2, wherein said at least one of said one or more polypeptides is selected from the group consisting of SEQ ID NO:79, SEQ ID NO:80, SEQ ID NO:86, and SEQ ID NO:87.

4. A pharmaceutical composition comprising an antibody or an antigen binding fragment thereof and a pharmaceutically acceptable carrier, wherein said antibody or antigen binding fragment thereof specifically recognizes at least one epitope consisting of 5 to 19 consecutive amino acids of SEQ ID NO: 84 including SEQ ID NO: 85, wherein said at least one epitope does not consist of 16 consecutive residues of SEQ ID NO:84.

5. The pharmaceutical composition of claim 4, wherein said antigen binding fragment is selected from the group consisting of Fab fragments, Fab′ fragments, F(ab′)₂ fragments, Fd fragments, Fv fragments, scFv fragments, and combinations thereof.

6. The pharmaceutical composition of claim 4, wherein said at least one epitope is selected from the group consisting of SEQ ID NO:79, SEQ ID NO:80, SEQ ID NO:86, and SEQ ID NO:87

7. A method of protecting or treating a subject from a zoonotic disease comprising the step of administering to said subject an immunogenic composition including one or more isolated polypeptides, wherein at least one of said one or more polypeptides consists of 5 to 19 consecutive residues of SEQ ID NO:84 including SEQ ID NO: 85, wherein said at least one of said one or more polypeptides does not consist of 16 consecutive residues of SEQ ID NO:84.

8. The method of claim 7, wherein said at least one of said one or more polypeptides is selected from the group consisting of SEQ ID NO:79, SEQ ID NO:80, SEQ ID NO:86, and SEQ ID NO:87.

9. The method of claim 7, wherein said zoonotic disease is caused by an obligate intracellular Anaplasmataceae bacterium selected from the group consisting of Anaplasma phagocytophilum and Anaplasma marginale.

10. The method of claim 9, wherein said subject is a cow and said zoonotic disease is bovine anaplasmosis.

11. A method of protecting or treating a subject from a zoonotic disease comprising the step of administering to said subject a pharmaceutical composition including an antibody or an antigen binding fragment thereof that specifically recognizes at least one epitope consisting of 5 to 19 consecutive amino acids of SEQ ID NO: 84 including SEQ ID NO: 85, wherein said at least one epitope does not consist of 16 consecutive residues of SEQ ID NO:84.

12. The method of claim 11, wherein said antigen binding fragment is selected from the group consisting of Fab fragments, Fab′ fragments, F(ab′)₂ fragments, Fd fragments, Fv fragments, scFv fragments, and combinations thereof.

13. The method of claim 11, wherein said at least one epitope is selected from the group consisting of SEQ ID NO:79, SEQ ID NO:80, SEQ ID NO:86, and SEQ ID NO:87.

14. The method of claim 11, wherein said zoonotic disease is caused by an obligate intracellular Anaplasmataceae bacterium selected from the group consisting of Anaplasma phagocytophilum and Anaplasma marginale.

15. The method of claim 14, wherein said subject is a cow and said zoonotic disease is bovine anaplasmosis.

16. A method of determining if a subject has been exposed to or is infected with an obligate intracellular Anaplasmataceae bacterium selected from the group consisting of Anaplasma phagocytophilum and Anaplasma marginale, wherein said subject is suspected of having a zoonotic disease caused by an obligate intracellular Anaplasmataceae bacterium, comprising the steps of
contacting a test sample from said subject, under conditions that allow polypeptide-antibody complexes to form, with a composition that includes one or more polypeptides, at least one of which consists of sixteen to nineteen consecutive residues of SEQ ID NO:84 including SEQ ID NO: 85, wherein said at least one of said one or more polypeptides does not consist of 16 consecutive residues of SEQ ID NO:84,
detecting one or more polypeptide-antibody complexes in said test sample, wherein the detection is an indication that antibodies specific for Anaplasmataceae OmpA are present in the test sample, and
determining said subject has been exposed to or is infected with said Anaplasmataceae bacterium if said antibodies specific for Anaplasmataceae OmpA are present in the test sample.

17. The method of claim 16, wherein said at least one of said one or more polypeptides is selected from the group consisting of SEQ ID NO:79, SEQ ID NO:80, SEQ ID NO:86, and SEQ ID NO:87.

18. The method of claim 16, wherein said contacting and detecting steps are performed using an assay selected from the group consisting of an immunoblot and an enzyme-linked immunosorbent assay (ELISA).

19. The method of claim 16, wherein said subject is a cow and said zoonotic disease is bovine anaplasmosis.

20. The method of claim 16, wherein said test sample is a body fluid selected from the group consisting of blood, plasma, serum, urine, and saliva.
